(19) United States
(12) Patent Application Publication
(10) Pub. No.: US 2019/0367964 A1
(43) Pub. Date: Dec. 5, 2019

(54) DISSOCIATION OF HUMAN TUMOR TO SINGLE CELL SUSPENSION FOLLOWED BY BIOLOGICAL ANALYSIS

(71) Applicants: DANA-FARBER CANCER INSTITUTE, INC., Boston, MA (US); MASSACHUSETTS INSTITUTE OF TECHNOLOGY, Cambridge, MA (US); THE BROAD INSTITUTE INC., Cambridge, MA (US); PRESIDENT AND FELLOWS OF HARVARD COLLEGE, Cambridge, MA (US)

(72) Inventors: Benjamin Izar, Cambridge, MA (US); Levi Garraway, Newton, MA (US); Asaf Rotem, Newton, MA (US); Aviv Regev, Cambridge, MA (US); Alexander Shalek, Cambridge, MA (US); Marc Wadsworth, Cambridge, MA (US); Sanjay Prakadan, Cambridge, MA (US); Orit Rozenblatt-Rosen, Cambridge, MA (US); Itay Tirosh, Cambridge, MA (US)

(73) Assignees: DANA-FARBER CANCER INSTITUTE, INC., Boston, MA (US); THE BROAD INSTITUTE, INC., Cambridge, MA (US); MASSACHUSETTS INSTITUTE OF TECHNOLOGY, Cambridge, MA (US); PRESIDENT AND FELLOWS OF HARVARD COLLEGE, Cambridge, MA (US)

(21) Appl. No.: 16/074,662

(22) PCT Filed: Feb. 2, 2017
(86) PCT No.: PCT/US2017/016146

§ 371 Date: Aug. 1, 2018

Related U.S. Application Data
(60) Provisional application No. 62/290,242, filed on Feb. 2, 2016.

(51) Int. Cl. C12Q 1/24; G01N 1/28; B01L 3/02; G01N 1/40; C12Q 1/6806

(52) U.S. Cl. CPC C12Q 1/24; G01N 1/286; B01L 3/0275; G01N 1/4077; G01N 2001/4088; G01N 2001/2873; B01L 2200/0605; B01L 2300/16; C12Q 1/6806

(57) ABSTRACT

Described herein are compositions and methods of disaggregating a tissue sample into single cells.

### RELATED APPLICATIONS

This application claims the benefit of priority under 35 U.S.C. § 119(e) to U.S. Provisional Application No. 62/290,242, filed Feb. 2, 2016, which is incorporated herein by reference in its entirety.

### GOVERNMENT LICENSE RIGHTS

This invention was made with government support under grant number 5R33CA155554-03 awarded by The National Institutes of Health and under grant number 5U01HG006492-03 awarded by The National Institutes of Health. The government has certain rights in the invention.

### BACKGROUND OF THE INVENTION

Tumors are complex ecosystems defined by spatiotemporal interactions between heterogeneous cell types, including malignant, immune, and stromal cells. Each tumor's cellular composition, as well as the interplay between these components, exerts critical roles in cancer development. Dissociation of patient-derived tumors allows for analysis of single cells. However, prior to the invention described herein, dissociation of tumors into single cells was a very long process that caused unwanted changes to the cellular genetic profile. As such, there is pressing need to develop short, effective methods of dissociating tumors into single cells without causing unwanted changes to the cellular genetic profile.

### SUMMARY OF THE INVENTION

The invention is based upon the identification of methods of dissecting the multicellular ecosystem of metastatic melanoma by single-cell ribonucleic acid (RNA)-seq. Specifically, described herein are methods for the dissociation of human tumor into a single cell suspension.

Provided are methods of disaggregating a tissue sample into a population of single cells in about one hour or less total. First, the tissue sample is dissected into pieces. Next, the tissue sample is enzymatically disaggregated for about 1 minute to about 20 minutes. Finally, the tissue sample is mechanically disaggregated by pipetting the tissue sample up and down for about 30 seconds to about 5 minutes, thereby disaggregating the tissue sample into a population of single cells in about one hour or less, wherein at least 50% to 100% of the single cells are viable and retain surface markers.

Also, methods of disaggregating a tissue sample into a population of single cells are carried out by dissecting the tissue sample into pieces; enzymatically disaggregating the tissue sample; and mechanically disaggregating the tissue sample, thereby disaggregating the tissue sample into a population of single cells. Optionally, the method further comprises performing single-cell RNA-seq on the single-cell sample.

The tissue sample is dissected into pieces less than 1 cm³. For example, the tissue sample is dissected into pieces <10 mm³, e.g., less than 9 mm³, less than 8 mm³, less than 7 mm³, less than 6 mm³, less than 5 mm³, less than 4 mm³, less than 3 mm³, less than 2 mm³, or less than 1 mm³. Preferably, the tissue sample is dissected into pieces <1 mm³, e.g., less than 0.1 mm³, less than 0.15 mm³, less than 0.01 mm³, or less than 0.001 mm³. Optionally, the tissue sample is dissected with a scalpel.

Following dissection, the tissue sample is enzymatically disaggregated with collagenase P and DNase I for about 10 minutes at about 37° C. Alternatively, the tissue sample is enzymatically disaggregated with AccuMax (Innovative Cell Technologies, Inc., San Diego, Calif.) for about 10 minutes at room temperature. In another example, the tissue sample is enzymatically disaggregated with collagenase IV and DNase I for about 10 minutes at about 37° C.

In some cases, the tissue sample is enzymatically disaggregated for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes or about 20 minutes. Preferably, trypsin is not utilized in the methods described herein.

After dissection and enzymatic disaggregation, the tissue sample is mechanically disaggregated by pipetting the tissue sample up and down. That is, the tissue sample is triturated or reduced to fine particles by mechanically moving the tissue sample up and down in the pipette. For example, the tissue sample is mechanically disaggregated by pipetting the tissue sample up and down for 1 minute with pipettes of descending sizes. In some cases, the tissue sample is mechanically disaggregated by pipetting the tissue sample up and down for about 30 seconds, about 1 minute, about 2 minutes, about 3 minutes, about 4 minutes, or about 5 minutes. For example, the pipettes comprise a 25 ml pipette, 10 ml pipette, 5 ml pipette, and 1 ml pipette. Other suitable pipettes include a 2 ml pipette and a 1,000 μl pipette tip. In some cases, the tissue sample is mechanically disaggregated by pipetting the tissue sample up and down for 1, 2, 3, 4, 5, 6, 7, 8, 9, 10, or additional minutes with pipettes of descending sizes. Optionally, the step of disaggregating by pipetting is repeated.

In some cases, the pipette diameter is progressively decreased with a removable pipette tip adapter. In one aspect, the pipette comprises an internal surface comprising teeth which mechanically shred the tissue sample.

In some cases, the method further comprises removing red blood cells from the tissue sample. For example, red blood cells are lysed with ammonium-chloride-potassium (ACK) lysing buffer.

Optionally, the method further comprises filtering the tissue sample and discarding residual cell clumps. Suitable filter sizes include 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, and 150 μm. For example, the tissue sample is filtered with a 70 μm nylon mesh filter or a 100 μm nylon mesh filter.

In one aspect, the population of cells comprises a single cell suspension. Preferably, at least 50% of the single cells are viable after performing the method, e.g., at least 55%, at least 60%, at least 65%, at least 70%, at least 75%, at least 80%, at least 85%, at least 90%, at least 95%, or 100% of the single cells are viable after performing the methods described herein to disaggregate a tissue sample into single cells. The methods described herein do not alter, remove, or add single cell surface markers.

Preferably, the method is performed in less than 5 hours, e.g., in less than 4 hours, in less than 3 hours, in less than two hours, or in less than 1 hour. Preferably, the method described herein is performed in less than 1 hour.

Exemplary tissue samples include cancer tissue, non-cancerous diseased tissue, and healthy normal tissue. In some cases, the tissue sample is derived from a melanoma, ovarian cancer, breast cancer, colorectal cancer, pancreatic cancer, lung cancer, head and neck cancer, or prostate cancer. Suitable tissue samples include solid tumor samples, core needle biopsy samples, fine needle aspiration samples, malignant effusion samples, bone marrow aspirate samples, and blood samples. Preferably, the tissue sample is a human or a mouse tissue sample. For example, the tissue sample comprises solid tissue, spheroid tissue, or a single cell solution. Suitable single cells isolated using the methods described herein include tumor cells, T-cells, B-cells, NK-cells, macrophages, dendritic cells, cancer-associated fibroblasts, or endothelial cells.

Also provided are kits comprising collagenase P, DNase I, and a pipette tip. Other kits include collagenase IV and DNase I and a pipette tip. For example, the kit comprises a 25 ml pipette tip, a 15 ml pipette tip, a 10 ml pipette tip, a 5 ml pipette tip, and a 1 ml pipette tip. In some cases, the kit further comprises a series of pipette tip adapters, wherein the pipette tip diameter is decreased. Optionally, the pipette tip adapter is produced utilizing a 3D printer. In some cases, the kit comprises a pipette tip comprising an internal surface comprising teeth for use in shredding a tissue sample. Optionally, the kit further comprises a scalpel.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “antineoplastic agent” is used herein to refer to agents that have the functional property of inhibiting a development or progression of a neoplasm in a human. Inhibition of metastasis is frequently a property of antineoplastic agents.

By “agent” is meant any small compound, antibody, nucleic acid molecule, or polypeptide, or fragments thereof.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

By “binding to” a molecule is meant having a physicochemical affinity for that molecule.

By “control” or “reference” is meant a standard of comparison. As used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled. Bridging moieties may amplify a detectable signal. For example, useful labels may include radioactive isotopes, magnetic beads, metallic beads, colloidal particles, fluorescent labeling compounds, electron-dense reagents, enzymes (for example, as commonly used in an enzyme-linked immunosorbent assay (ELISA)), biotin, digoxigenin, or haptens. When the fluorescently labeled molecule is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as 152 Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

A “detection step” may use any of a variety of known methods to detect the presence of nucleic acid. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the term “disaggregate” is meant to separate something into its component parts. Thus, “disaggregating” a tissue sample into a population of single cells means to separate a tissue sample into the single cells which together form the tissue sample.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., cancer, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “expression profile” is used broadly to include a genomic expression profile. Profiles may be generated by any convenient means for determining a level of a nucleic acid sequence, e.g., quantitative hybridization of microRNA, labeled microRNA, amplified microRNA, complementary/synthetic DNA (cDNA), etc., quantitative polymerase chain reaction (PCR), and ELISA for quantitation, and allow the analysis of differential gene expression between two samples. A subject or patient tumor sample is assayed. Samples are collected by any convenient method, as known in the art. According to some embodiments, the term “expression profile” means measuring the relative abundance of the nucleic acid sequences in the measured samples.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “immobilized” or “attached” refers to a probe (e.g., nucleic acid or protein) and a solid support in which the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule to the support and the non-covalent binding of a biotinylated probe to the molecule. Immobilization may also involve a combination of covalent and non-covalent interactions.

By “marker” is meant any protein or polynucleotide having an alteration in expression level or activity that is associated with a disease or disorder, e.g., cancer.

By “modulate” is meant alter (increase or decrease). Such alterations are detected by standard art-known methods such as those described herein.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia, e.g., cancer, and of its evolution as well as the prospect of recovery as anticipated from usual course of the disease.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

The term “sample” as used herein refers to a biological sample obtained for the purpose of evaluation in vitro. Exemplary tissue samples for the methods described herein include tissue samples from tumors or the surrounding microenvironment (i.e., the stroma). With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Other samples include whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer (e.g., cancer) is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

Any compositions or methods provided herein can be combined with one or more of any of the other compositions and methods provided herein.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1D are a series of schematics and graphs showing dissection of melanoma using single-cell RNA-seq. FIG. 1A shows an overview of workflow. FIG. 1B shows that chromosomal landscape of inferred large-scale copy number variations (CNVs) distinguishes malignant from non-malignant cells. One example tumor (Mel80) is shown with individual cells (y-axis) and chromosomal regions (x-axis). Amplifications (red) or deletions (blue) were inferred by averaging expression over 100-gene stretches on the respective chromosomes. Inferred CNVs are strongly concordant with calls from whole-exome sequencing (WES, bottom). FIG. 1C and FIG. 1D show that single cell expression profiles distinguish malignant and non-malignant cell types. Shown are t-SNE plots of malignant (FIG. 1C, shown are the six tumors each with >50 malignant cells) and non-malignant (FIG. 1D) cells (as called from inferred CNVs as in FIG. 1B) from 11 tumors with >100 cells per tumor (color code). Clusters of non-malignant cells (called by DBScan) are marked by dashed ellipses and were annotated as T cells, B cells, macrophages, CAFs and endothelial cells, based on preferentially expressed genes (FIG. 7 and Table 2-3).

FIG. 2A-FIG. 2D are a series of graphs which show that single-cell RNA-seq distinguishes cell cycle and other states among malignant cells. FIG. 2A shows an estimation of the cell cycle state of individual malignant cells (circles) based on relative expression of G1/S (x-axis) and G2/M (y-axis) gene-sets in a low-cycling (Mel79, top) and a high-cycling (Mel78, bottom) tumor. Cells are colored by their inferred cell cycle states, with cycling cells (red), intermediate (bright red) and non-cycling cells (black); cells with high expression of KDM5B (Z-score>2) are marked in cyan filling. FIG. 2B shows immunohistochemistry (IHC) staining (40× magnification) for Ki67+ cells shows a high concordance with the signature-based frequency of cycling cells for Mel79 and Mel78 (as for other tumors; FIG. 9C). FIG. 2C shows KDM5B/Ki67 staining (40× magnification) in corresponding tissue showing small clusters of KDM5B-high expressing cells that are all negative for Ki67 (see also FIG. 9). FIG. 2D shows an expression program specific to Region 1 of Mel79, based on multifocal sampling. The relative expression of genes (rows) is shown for cells (columns) ordered by the average expression of the entire gene-set. The region-of-origin of each cell is indicated in the top panel (see also FIG. 10).

FIG. 3A-FIG. 3F is a series of graphs showing that MITF- and AXL-associated expression programs vary between tumors, within tumors, and following treatment. FIG. 3A that shows average expression signatures for the AXL program (y-axis) or the MITF program (x-axis) stratify tumors into ‘MITF-high’ (black) or ‘AXL-high’ (red). FIG. 3B shows single-cell profiles which show a negative correlation between the AXL program (y-axis) and MITF program (x-axis) across individual malignant cells within the same tumor; cells are colored by the relative expression of the MITF (black) and AXL (red) programs. Cells in both states are found in all examined tumors, including three tumors (Mel79, Mel80 and Mel81) without prior systemic treatment, indicating that dormant resistant (AXL-high) cells may already be present in treatment naïve patients. FIG. 3C shows Mel81 and Mel80 immunofluorescence staining of MITF (green nuclei) and AXL (red), validating the mutual exclusivity among individual cells within the same tumor (see also FIG. 13). FIG. 3D shows relative expression (centered) of the AXL-program (top) and MITF-program (bottom) genes in six matched pre-treatment (white boxes) and post-relapse (gray boxes) samples from patients who progressed through RAF/MEK inhibition therapy; numbers at the top indicate patient index. Samples are sorted by the average relative expression of the AXL vs. MITF gene-sets. In all cases, the relapsed samples had increased ratio of AXL/MITF expression compared to their pre-treatment counterpart. This consistent shift of all six patients is statistically significant (P<0.05, binomial test), as are the individual increases in AXL/MITF for four of the six sample pairs (P<0.05, t-test; black and gray arrows denote increases that are individually significant or non-significant, respectively). FIG. 3E shows flow-cytometric quantification of the relative fraction of cells with AXL-high (log-scale, y-axis) expression, when cells were treated with increasing doses of RAF/MEK-inhibition (dabrafenib and trametinib in a 10:1 ratio at indicated doses). In all examined cell lines (x-axis), there was a dose-dependent increase in the AXL-high expressing cell fraction. FIG. 3F shows quantitative, multiplexed single-cell immunofluorescence for AXL expression (y-axis top), MAP-kinase pathway inhibition (pERK levels, y-axis) and viability (y-axis bottom) in the example cell line WM88 treated with increasing concentrations (y-axis) of either RAF inhibitor alone (black bars) or a combination of RAF/MEK-inhibitors (yellow bars). Increasing relative AXL-high expressing cell fraction (top panel), consistent with flow-cytometry, as well as a dose-dependent decrease of p-ERK (middle) and viability (bottom), overall consistent with phenotypic selection (killing of MITF-high cells) as part of the shift towards the AXL-high fraction (see FIG. 16-FIG. 17 for additional cell lines) was observed.

FIG. 4A-FIG. 4D is a series of graphs which show that deconvolution of bulk melanoma profiles by specific signatures of non-cancer cell types reveals cell-cell interactions. FIG. 4A shows that bulk tumors segregate to distinct clusters based on their inferred cell type composition. Top panel: heat map showing the relative expression of gene sets defined from single-cell RNA-seq as specific to each of five cell types from the tumor microenvironment (y-axis) across 495 melanoma TCGA bulk-RNA signatures (x-axis). Each column is one tumor and tumors are partitioned into 10 distinct patterns identified by K-means clustering (vertical lines and cluster numbers at the top). Lower panels show from top to bottom tumor purity, specimen location (from TCGA), and AXL/MITF scores. Tumor purity as estimated by the expression of cell-type specific gene-sets (“RNA”) was strongly correlated with that estimated by ABSOLUTE mutation analysis (“DNA”, R=0.8, bottom panel, both smoothed with a moving average of 40 tumors). Tumor classification, and in particular tumors with high abundance of CAFs, is strongly correlated with an increased ratio of AXL-program/MITF-program expression (bottom). FIG. 4B shows inferred cell-to-cell interactions between CAFs and T cells. Scatter plot compares for each gene (circle) the correlation of its expression with inferred T cell abundance across bulk tumors (y-axis, from TCGA transcriptomes) to how specific its expression is to CAFs vs. T cells (x-axis, based on single-cell transcriptomes). Genes that are highly specific to CAFs in a single cell analysis of tumors (red), but also associated with high T cell abundance in bulk tumors (black border) are key candidates for CAF/T cell interactions. This analysis identified known (CXCL12, CCL19) genes linked to immune cell chemotaxis and putative immune modulators, including multiple complement factors (C1R, C1S, C3, C4A, CFB and C1NH [SERPING1]). FIG. 4C shows a correlation between quantitative immunofluorescence signal (% Area) of C3 and CD8 levels across 308 core biopsies of melanoma tissue microarrays. Shown are 90 included samples with 80 tumor specimens (black dots) showing a correlation (R=0.86) between C3/C8 signal and 10 normal control specimens (grey dots). See FIG. 23A-FIG. 23F for normalization and additional specimens. FIG. 4D shows a correlation coefficient (y-axis) between the average expression of CAF-derived complement factors shown in FIG. 4B and that of T cell markers (CD3/D/E/G, CD8A/B) across 26 TCGA cancer types with >100 samples (x-axis, left panel) and across 36 GTEx tissue types with >100 samples (x axis, right panel). Bars are colored based on correlation ranges as indicated at the bottom.

FIG. 5A-FIG. 5H are graphs that show that T cell analysis distinguishes activation-dependent and independent variation in coexpressed exhaustion markers. FIG. 5A shows single T cell stratification into CD4+ and CD8+ cells (upper panel), CD25+FOXP3+ and other CD4 cells (middle panel) and their inferred activation state (lower panel, based on average expression of the cytotoxic and naïve gene-sets shown in (FIG. 5B)). FIG. 5B shows tgat average expression of markers of cytotoxicity, exhaustion and naïve cell states (rows) in (left to right) Tregs, CD4+ T cells, and CD8+ T cells; CD4+ and CD8+ T cells are each further divided into five bins by their cytotoxic score (ratio of cytotoxic to naïve marker expression levels), showing an activation-dependent co-expression of exhaustion markers. Bottom: proportion of cycling cells (calculated as in FIG. 2B). Asterisks denote significant enrichment or depletion of cycling cells in a specific subset compared to the corresponding set of CD4+ or CD8+ T cells (P<0.05, hypergeometric test). FIG. 5C shows immunofluorescence of PD-1 (upper panel, green), TIM-3 (middle panel, red) and their overlay (lower panel) validates their co-expression. FIG. 5D shows activation-independent variation in exhaustion states within highly cytotoxic T cells. Scatter plot shows the cytotoxic score (x-axis) and exhaustion score (y-axis, average expression of the Mel75 exhaustion program shown in FIG. 26) of each CD8+ T cell from Mel75. In addition to the overall correlation between cytotoxicity and exhaustion, the cytotoxic cells can be sub-divided into highly exhausted (red) and lowly exhausted cells (green) based on comparison to a LOWESS regression (black line). FIG. 5E-FIG. 5F show relative expression (log 2 fold-change) in high vs. low exhaustion cytotoxic CD8+ T cells from five tumors (x-axis), including 28 genes that were significantly induced (P<0.05, permutation test) in high-exhaustion cells across tumors (FIG. 5E) and 272 genes that were variably expressed across tumors (FIG. 5F). Three independently derived exhaustion gene-sets were used to define high and low exhaustion cells (Mel75, (Wherry et al., 2007 Immunity, 27: 670-684; L. Baitsch et al., 2012 PLoS ONE. 7, e30852)), and the corresponding results are represented as distinct columns for each tumor. FIG. 5G shows expanded TcR clones. Cells were assigned to clusters of TCR segment usage (black bars; FIG. 28), and cluster size (x-axis) was evaluated for significance by control analysis in which TCR segments were shuffled across cells (grey bars). The percentage of Mel75 cells (y-axis) is shown for clusters of small size (1-4 cells) that likely represent non-expanded cells, medium size (5-6 cells) that may reflect expanded clones (FDR=0.12), and large size that most likely reflect expanded clones (FDR=0.005). FIG. 5H shows that expanded clones are depleted of nonexhausted cells and enriched for exhausted cells. Mel75 cells were divided by exhaustion score into low exhaustion (green, bottom 25% of cells) and medium-to-high exhaustion (red, top 75%). Shown is the relative frequency of these exhaustion subsets (y-axis) in each TCR-cluster group (x-axis, as defined in G), defined as log 2-ratio of the frequency in that group compared to the frequency across all Mel75 cells. All values were highly significant (P<10-5, binomial test).

FIG. 6A-FIG. 6B is a series of graphs showing classification of cells to malignant and non-malignant based on inferred CNV patterns. FIG. 6A shows the same as shown in FIG. 1B for another melanoma tumor (Mel78). FIG. 6B is a graph wherein each plot compares two CNV parameters for all cells in a given tumor: (1) CNV score (X-axis) reflects the overall CNV signal, defined as the mean square of the CNV estimates across all genomic locations; (2) CNV correlation (Y-axis) is the Pearson correlation coefficient between each cell's CNV pattern and the average CNV pattern of the top 5% of cells from the same tumor with respect to CNV signal (i.e., the most confidently-assigned malignant cells). These two values were used to classify cells as malignant (red; CNV score >0.04; correlation score >0.4; grey lines mark thresholds on plot), non-malignant (blue; CNV score <0.04; correlation score <0.4), or unresolved intermediates (black, all remaining cells). In four tumors (Mel58, 67, 72 and 74), primarily the immune infiltrates (CD45− cells) were sequenced and there were only zero or one malignant cells by this definition; in those cases, CNV correlation is not indicative of malignant cells (since the top 5% cells by CNV signal are primarily non-malignant) and therefore all cells except for one in Mel58 were defined as non-malignant. Note that while these thresholds are somewhat arbitrary, this classification was highly consistent with the clustering patterns of these cells (as shown in FIG. 1C) into clusters of malignant and non-malignant cells.

FIG. 7A-FIG. 7I is a series of graphs showing the identification of non-malignant cell types by tSNE clusters that preferentially express cell type markers. FIG. 7A-FIG. 7H is a series of graphs wherein each plot shows the average expression of a set of known marker genes for a particular cell type (as indicated at the top) overlaid on the tSNE plot of non-malignant cells, as shown in FIG. 1C. Gray indicates cells with no or minimal expression of the marker genes (FIG. 7E, average log 2 (TPM+1), below 4), dark red indicates intermediate expression (4<E<6), and light red indicates cells with high expression (E>6). FIG. 7I shows DBscan clusters derived from tSNE coordinates, with parameters eps=6 and min-points=10. Eleven clusters are indicated by numbers and colors.

FIG. 8A and FIG. 8B are graphs which show limited influence of tumor site on RNA-seq patterns. FIG. 8A and FIG. 8B are heat maps which show correlations of global expression profiles between tumors, which were ordered by metastatic site. Expression levels were first averaged over melanoma (FIG. 8A) or T cells (FIG. 8B) in each tumor and then centered across the different tumors before calculating Pearson correlation coefficients. Differential expression analysis conducted between the two groups of tumors found zero differentially expressed genes with FDR of 0.05 based on a shuffling test for both T cells and melanoma cells.

FIG. 9A-FIG. 9E is a series of graphs showing the identification and characterization of cycling malignant cells. (A) Heat map showing relative expression of G1/S (top) and G2/M (bottom) genes (rows, as defined from integration of multiple datasets) across cycling cells (left panel, columns, ordered by the ratio of expression of G1/S genes to G2/M genes) and across all cells (right panel, columns, cycling cells ordered as in left panel followed by non-cycling cells at random order). Cycling cells were defined as those with significantly high expression of G1/S and/or G2/M genes (FDR<0.05 by t-test, and fold-change >4 compared to all malignant cells). FIG. 9B shows the frequency of inferred cycling cells (Y axis) in seven tumors (X axis) with >50 malignant cells/tumors, denoting low (<3%) or high (>20%) proliferation tumors. FIG. 9C (upper panel) shows a significant correlation (P<0.038) between inferred proportion of cycling cells by single-cell transcriptome analysis (horizontal axis) and Ki67+ immunohistochemistry (IHC) (lower panel) of corresponding tumor slides (vertical axis). FIG. 9D shows a comparison of cycling cell expression programs between low- and high-proliferation tumors. Scatter plots compared the expression log-ratio between cycling and non-cycling cells in high-proliferation (y-axis) and low-proliferation (x-axis) tumors. Genes significantly upregulated (P<0.01, fold-change >2) in cycling cells in both types of tumor are marked in red. CCND3 (arrow) is significantly upregulated in cycling cells in high-proliferation tumors and downregulated in cycling cells in low-proliferation tumors. FIG. 9E shows dual KDMSB (JARID1B)/Ki67 immunofluorescence staining of tissue slide of Mel80 (40× magnification). Consistent with findings presented for Mel78 and Mel79 in FIG. 2C, KDMSB-expressing cells (green nuclear staining) occurred in small clusters of two or more cells and do not express Ki67 (red nuclear staining), indicating that these cells are not undergoing cell division.

FIG. 10A-FIG. 10B are photomicrographs showing immunohistochemistry of melanoma 79 shows gross differences between tumor parts and increased NF-κB levels in Region 1. FIG. 10A shows tumor dissection into five regions. Left: melanoma tumor prior to dissection. Macroscopically distinct regions are highlighted by colored ovals. Right: The tumor was dissected into five pieces, which were further processed as individual samples. Regions 1, 3, 4 and 5 were included in the single-cell RNA-seq analysis; Cells from Region 2 were lost during library construction. FIG. 10B shows a corresponding histopathological cross-section of the tumor demonstrates distinct features of Region 1 compared to the other regions. Consistent with enrichment of cells in Region 1 expressing multiple markers that are highlighted in FIG. 2D, immunohistochemistry staining revealed increased staining of NF-κB and JunB in Region 1 (right lower panel, 40× magnification), compared to Region 3 (right upper panel, 40× magnification).

FIG. 11A-FIG. 11B are graphs showing spatial heterogeneity in the expression of CD8+ T-cells. As shown in FIG. 2D for malignant cells, the expression differences between regions of Mel79 for other cell types were examined. The only cell type for which there were >10 cells in each of the regions was CD8+ T cells. The differences among CD8+ T cells were examined and 62 genes that were preferentially expressed in region 1 (fold-change>2, FDR<0.05) and that partially overlapped the region 1-specific genes among the malignant cells (see Table 6) were identified. FIG. 11A shows a region 1-specific expression program of CD8+ T-cells (as shown in FIG. 2D for malignant cells). Bottom: heat map shows the relative expression of the 62 genes preferentially expressed in region 1, in all CD8+ T-cells from Mel79, ranked by their average expression of these genes. A subset of genes of interest are noted at the right. Top: assignment of cells to the four regions of Mel79. FIG. 11B shows a comparison of region 1 preferential expression between malignant cells (X-axis) and CD8+ T-cells (Y-axis). For each cell type, the scatterplot shows the log 2-ratio between the average expression of all cells in region 1 and those in all other regions.

FIG. 12 is a series of dot plots that show intra-tumor heterogeneity in AXL and MITF programs. AXL-program (Y-axis) and MITF-program (X-axis) scores for malignant cells in each of the three tumors with a sufficient number of malignant cells (n>50) that were not included in FIG. 3B. Cells are colored from black to red by the relative AXL and MITF scores. The Pearson correlation coefficient is denoted on top.

FIG. 13 is a series of photomicrographs showing that AXL/MITF immunofluorescence staining of tissue slides of Mel80, Mel81 and Mel79 (40× magnification) revealed presence of AXL-expressing and MITF-expressing cells in each sample. Consistent with single-cell RNA-seq inferred frequencies of each population, Mel80 contained rare AXL-expressing cells (red, cell membrane staining) and mostly malignant MITF-positive cells (green, nuclear staining), while malignant cells of Mel81 almost exclusively consisted of AXL-expressing cells. Mel79 had a mixed population with rare cells positive for both markers, all in agreement with the inferred single-cell transcriptome data.

FIG. 14 is a dot plot showing AXL upregulation in a second cohort of post-treatment melanoma samples and mutual exclusivity with MET upregulation. Each point reflects a comparison between a matched pair of pre-treatment and post-relapse samples from Hugo et al., 2015 Cell, 162: 1271-1285, where the X-axis shows expression changes in MET, and the Y-axis shows expression changes in the AXL program minus those of the MITF program. Note that some patients are represented more than once based on multiple post-relapse samples. Fourteen out of 41 samples (34%) shown in red had significant upregulation of the AXL vs. MITF program, as determined by a modified t-test as described in Example 1; these correspond to at least one sample from half (9/18) of the patients included in the analysis. Eleven out of 41 samples (27%) shown in blue had at least 3-fold upregulation of MET; these correspond to at least one sample from a third (6/18) of the patients included in the analysis. Notably, the AXL and MET upregulated samples are mutually exclusive, consistent with the possibility that these are alternative resistance mechanism.

FIG. 15A is a series of graphs showing flow cytometry gating strategy for the exemplary cell lines WM88 (AXL-low) and IGR39 (AXL-high). Cells were treated with increasing doses of dabrafenib (D) and trametinib (T) at indicated doses, which resulted in an increase in the AXL-high cell fraction in WM88, and no changes in IGR39. FIG. 15B is a bar chart showing while cell lines with very low portion of AXL-positive cells demonstrate an increased frequency of AXL-high cells (FIGS. 3E and F) with combined BRAF/MEK-inhibition, AXL-high cell lines show minimal to no changes.

FIG. 16A-FIG. 16C is a series of bar charts showing a summary of multiplexed single-cell immunofluorescence in seven CCLE cell lines before and after treatment with BRAF/MEK-inhibition. FIG. 16A shows relative fraction (compared to DMSO-treatment) of AXL-high cells (y-axis) treated for 5 or 10 days with increasing doses (as indicated on x-axis) of BRAF-inhibition alone (with vemurafenib) or in combination with a MEK-inhibitor (trametinib) with a 10:1 ratio (vemurafenib:trametinib). In all cell lines with a baseline low-fraction of AXL-expressing cells (WM88, MELHO, COL0679 and SKMEL28), there was a significant dose-dependent increase in the AXL-high cell fraction with BRAF-inhibition alone (black bars), and more pronounced with combined BRAF/MEK-inhibition (yellow bars). Cell lines with a baseline high AXL-expressing cell fraction (A2058, IGR39 and 294T) showed either minimal changes in the AXL-high cell fraction; however, A2058 demonstrated a significant decreased in the AXL-positive fraction. Although an outlier in this experiment, this indicates that alternative mechanisms of resistance with low AXL expression (Hugo et al., 2015 Cell, 162: 1271-1285; FIG. 14). FIG. 16B shows that the increase in AXL-high cell fractions in the sensitive cell lines was correlated with a significant decrease of p-ERK indicating strong MAP-kinase pathway inhibition, and (FIG. 16C) a decrease in cell viability. Overall, these results indicate that the increase in the AXL-high cell fraction was at least in part due to a selection process. Both effects were more pronounced when cells were treated with combined BRAF/MEK-inhibition compared BRAF-inhibition alone.

FIG. 17A-FIG. 17B is a series of photomicrographs which show exemplary images of multiplexed single-cell immunofluorescence quantitative analysis for (FIG. 17A) an AXL-low (WM88) and (FIG. 17B) AXL-high cell line (A2058). Treatment with a combination of vemurafenib (V) and trametinib (T) at indicated doses on the left resulted in a dose-dependent change in the AXL-high population. In WM88, increasing drug concentrations led to killing of MITF-expressing, resulting in the emergence of a pre-existing AXL-high subpopulation. This indicates that the shift towards a higher AXL-expressing population (and possibly the AXL-high signature) is at least in part due to a selection process. While cell lines with a high baseline fraction of AXL-expressing cells showed modest to no changes in the AXL-fraction (FIG. 15B), A2058 was an exception. This cell lines has a major AXL-expressing population at baseline, which decreases with treatment, while the MITF-expressing population emerges. This indicates the presence of alternative mechanisms of resistance to RAF/MEK-inhibition, consistent with a recent report by Hugo et al., 2015 Cell, 162: 1271-1285 and the analysis shown in FIG. 14.

FIG. 18 is a graph showing the identification of cell-type specific genes in melanoma tumors. Shown are the cell-type specific genes (rows) as chosen from single cell profiles (Example 1), sorted by their associated cells cell type, and their expression levels (loge (TPM/10+1)) across non-malignant and malignant tumor cells, also sorted by type (columns).

FIG. 19A-FIG. 19B are a series of bar charts and a dot plot that show the association between a malignant AXL program and CAFs. FIG. 19A shows the average expression (log₂(TPM+1)) of the AXL program (Y-axis) as defined here (bottom) and by Hoek et al., 2008 Cancer Res, 68: 650-656 (top) in CAFs and melanoma cells from the tumors (this work, black bars) and in foreskin melanocytes and primary fibroblasts from the Roadmap Epigenome project (grey bars). Melanoma cells were partitioned to those from AXL-high and MITF-high tumors as marked in FIG. 3A. FIG. 19B shows that CAF expression correlates with higher AXL program than MITF program expression in melanoma malignant cells. Scatter plot shows for each gene (dot) from the MITF (blue) or AXL (red) programs (as defined based on single-cell transcriptomes) the correlation of its expression with inferred CAF frequency across bulk tumors (Y-axis, from TCGA transcriptomes), and how specific its expression is to CAFs vs. melanoma malignant cells (X-axis, based on single-cell transcriptomes). Black dots indicate the expected correlations at each value of the horizontal axis as defined by a LOWESS regression over all genes. The average correlation values of MITF program genes are significantly lower than those of all genes and the correlation values of A×L program genes are significantly higher than those of all genes, even after restricting the analysis to melanoma-specific genes (X-axis<−2, P<0.01, t-test). A subset of AXL-program genes are specifically expressed in melanoma cells (but not CAFs) based on the single cell expression profiles, but associated with CAF abundance in bulk tumors (marked by red squares and gene names). MITF is negatively correlated with CAF abundance (R=−0.42) and is also indicated by gene name.

FIG. 20A-FIG. 20C is a series of graphs showing the identification of putative genes underlying cell-to-cell interactions from analysis of single cell profiles and TCGA samples. Genes were searched that underlie potential cell-to-cell interactions, defined as those that are primarily expressed by cell type M (as defined by the single cell data), but correlate with the inferred relative frequency of cell type N (as defined from correlations across TCGA samples). For each pair of cell types (M and N), the analysis was restricted to genes that are at least four-fold higher in cell type M than in cell type N and in any of the other four cell types. Then the Pearson correlation coefficient (R) between the expression of each of these genes in TCGA samples and the relative frequency of cell type N in those samples was calculated, and these were converted into Z-scores. The set of genes with Z >3 and a correlation above 0.5 was defined as potential candidates that mediate an interaction between cell type M and cell type N. FIG. 20A shows that of all the pairwise comparisons interactions were identified only between immune cells (B, T, macrophages) and non-immune cells (CAFs, endothelial cells, malignant melanoma), such that the expression of genes from non-immune cells correlated with the relative frequency of immune cell types. Each plot shows a single pairwise comparison (M vs. N), including interactions of non-immune cell types (endothelial cells: left; CAFs: middle; malignant melanoma: right) with each of T-cells (FIG. 20A), B-cells (FIG. 20B) and macrophages (FIG. 20C). Each plot compares for each gene (dot) the relative expression of genes in the two cell types being compared (M-N) and the correlations of these genes' expression with the inferred frequency of cell type N across bulk TCGA tumors. Dashed lines denote the four-fold threshold. Genes that may underlie potential interactions, as defined above, are highlighted.

FIG. 21A-FIG. 21C are a series of graphs that show immune modulators expressed by CAFs and macrophages. FIG. 21A shows the Pearson correlation coefficient (color bar) across TCGA melanoma tumors between the expression level of each of the immune modulators shown in FIG. 4B and additional complement factors with significant expression levels. FIG. 21B shows correlations across TCGA melanoma tumors between the expression level of the genes shown in FIG. 21A and the average expression levels of T cell marker genes. FIG. 21C shows the average expression level (log₂ (TPM+1), color bar) of the genes shown in FIG. 21A in the single cell data, for cells classified into each of the major cell types identified. These results show that most complement factors are correlated with one another and with the abundance of T cells, even though some are primarily expressed by CAFs (including C3) and others by macrophages. In contrast, two complement factors (CFI, C5) and the complement regulatory genes (CD46 and CD55) show a different expression pattern.

FIG. 22A-FIG. 22C are a series of graphs that show the unique expression profiles of in vivo CAFs. FIG. 22A-FIG. 22B show distinct expression profiles in in vivo and in vitro CAFs. Shown are Pearson correlation coefficient between individual CAFs isolated in vivo from seven melanoma tumors, and CAFs cultured from one tumor (melanoma 80). Hierarchical clustering shows two clusters, one consisting of all in vivo CAFs, regardless of their tumor-of-origin (marked in FIG. 22A), and another of the in vitro CAFs. FIG. 22 C shows unique markers of in vivo CAFs include putative cell-cell interaction candidates. Left: Heatmap shows the expression level (log 2 (TPM+1)) of CAF markers (bottom) and the top 14 genes with higher expression in in-vivo compared to in-vitro CAFs (t-test). Right: average (bulk) expression of the genes in the in-vivo CAFs, in-vitro CAFs, and primary foreskin fibroblasts from the Roadmap Epigenome project. Potential interacting genes from FIG. 4B are highlighted in bold red.

FIG. 23A-FIG. 23F are a series of dot plots that show TMA analysis of complement factor 3 association with CD8+ T-cell infiltration, and control staining. Two TMAs (CC38-01 and ME208, shown in FIG. 23A, FIG. 23C, FIG. 23E and FIG. 23B, FIG. 23D, FIG. 23F, respectively) were used to evaluate the association between complement factor 3 (C3) and CD8 across a large number of tissues obtained by core biopsies of normal skin, primary tumors, metastatic lesions and NATs (normal skin with adjacent tumor). In both TMAs with a total of 308 core biopsies, a high correlation was observed between C3 and CD8 (R >0.8, shown in FIG. 4C for one TMA). To verify that this correlation is not due to technical effects in which some tissues stain more than others irrespective of the stains examined (e.g., due to variability in cellularity or tissue quality), the values were normalized (% area, Example 1) for both C3 and CD8 by those of DAPI staining. Indeed, a non-random yet non-linear association was identified between DAPI stains and either C3 (FIG. 23A, FIG. 23B), or CD8 (FIG. 23C, FIG. 23D), which were removed by subtracting a LOWESS regression, shown as red curves in panels FIG. 23A-FIG. 23D. The normalized C3 and CD8 values were not correlated with DAPI levels, yet maintained a high correlation with one another (FIG. 23E, FIG. 23F). R=0.86 and 0.74 for primary and normal skin in panel E (TMA CC38-01), and R=0.78, 0.86, 0.63 and 0.31 for primary melanomas, metastasis, NATs and normal skin in panel F (TMA ME208), respectively.

FIG. 24A and FIG. 24B are plots showing cytotoxic and naïve expression programs in T cells. FIG. 24A shows cell scores from a combined PCA of all T cells. Cells are colored as CD8+ (red), CD4+ (green), T-regs (blue) and unresolved (black) based on expression of marker genes (FIG. 5A, Example 1). FIG. 24B shows gene scores for PCI from a PCA of CD8+ cells (x-axis) and PC2 from a PCA of CD4+ cells (Y-axis). Selected marker genes are highlighted, including genes known to be associated with cytotoxic/active (red), naïve (blue) and exhausted (green) T cell states.

FIG. 25 shows the frequency of cycling cells in different subsets of T-cells. Shown is the frequency of cycling T cells (as identified based on the expression of G1/S and G2/M gene-sets; Example 1) for different subsets of T cells, including Tregs, CD4+ cells separated into five bins of increasing activation (arrow below green bars), CD8+ cells separated into five bins of increasing activation (arrow below red bars), and active/cytotoxic CD8+ further partitioned into those with relatively high or low exhaustion, as shown in FIG. 5D. Asterisks denote subsets with significant enrichment or depletion of cycling cells across all cells from the same subset of CD4+ or CD8+ cells as defined by P<0.05 in a hypergeometric test. Cell cycle frequency is associated with activation state of CD8+ T-cells, as the first bin is significantly depleted and the fifth bin is significantly enriched. A similar trend is observed in CD4+ T-cells (no cycling cells in the first bin and highest frequency in fifth bin), although none of the CD4 bins was significantly depleted or enriched. Exhaustion was not associated with significant differences in cell cycle frequency (P=0.34, Chi-square test).

FIG. 26A-FIG. 26B is a series of graphs showing the exhaustion program in Mel75. PCA of 314 CD8 T-cells from Mel75 identified an exhaustion program in which the top scoring genes for PC1 included the five co-inhibitory receptors shown in FIG. 5B as well as additional exhaustion-associated genes (e.g., BTLA, CBLB). PC1-associated genes were defined based on a correlation p-value of 0.01 (with Bonferroni correction for multiple testing, see Table 12). Cells were then ranked by the residual between average expression of these PC1-associated genes (referred to as the exhaustion program) and average expression of the cytotoxic genes shown in FIG. 5B (referred to as the cytotoxic program) using a LOWESS regression, as shown in FIG. 5D. Finally, for each gene, its expression levels were ranked across the CD8 T-cells from Mel75 and converted these to rank scores between 0 and 1 such that the i highest-expressing cell received a rank score of i/314, where 314 represents the number of CD8 T cells from Mel75. FIG. 26A shows exhaustion and cytotoxic program scores for ranked Mel75 CD8 T-cells, after applying a moving average with windows of 31 genes. FIG. 26B is a heatmap that shows expression ranks of PC1-associated genes across the CD8 T-cells from Mel75 cells, ranked as described above.

FIG. 27A-FIG. 27E are a series of graphs showing tumor-specific exhaustion programs. FIG. 27A is a heatmap showing the significance (−log 10(P-value)) of tumor-specific variation in exhaustion gene scores (log-ratio in high vs. low exhaustion cells) comparing each tumor to all other tumors combined, for the same genes (and the same order) as shown in FIG. 5F. The sign of significance values reflects the direction of change (positive values shown in red reflect higher exhaustion values compared to other tumors while negative values shown in green reflect lower exhaustion values compared to other tumors). Three values are shown for each tumor, corresponding to exhaustion scores based on the exhaustion gene-sets derived from Mel75 analysis (Baitsch et al., 2012 PLoS ONE. 7, e30852; Wherry et al., 2007 Immunity, 27: 670-684), respectively. FIG. 27B shows the number of genes with significant tumor-specific up- or down-regulation (FDR <0.05 in each tumor, based on median of the three exhaustion scores), divided to three classes (bars) based on the differences in overall expression level across CD8 T-cells of the different tumors (green: genes lower in the respective tumor by at least two fold. Red: genes higher in the respective tumor by at least two fold. Black: genes with less than two-fold difference. This demonstrates that most changes in exhaustion co-expression are not identified in bulk level analysis of the CD8 T-cells. FIG. 27C-FIG. 27D are bar plots showing the significance of tumor-specific variation, as in FIG. 27A, for CTLA4 (FIG. 27C) and NFATC1 (FIG. 27D). Dashed lines indicate significance thresholds that correspond to P<0.05. FIG. 27E is a heatmap (as in subfigure FIG. 27A) for the target genes of NFATC1 (Martinez et al., 2015 Immunity, 42: 265-278).

FIG. 28A and FIG. 28B are graphs showing detection of Mel74 expanded T-cell clones by TCR sequence. FIG. 28A shows clustering of Mel75 cells by their TCR segment usage. TCR Similarity was defined as zero for any pair with at least one inconsistent allele (i.e. resolved in both cells but distinct among the two cells), and as −log 10(P) for any pair without inconsistent alleles, where P reflects the estimated probability of randomly observing this or a higher degree of segment usage similarity. P is equal to the product of the probabilities for the four TCR segments,

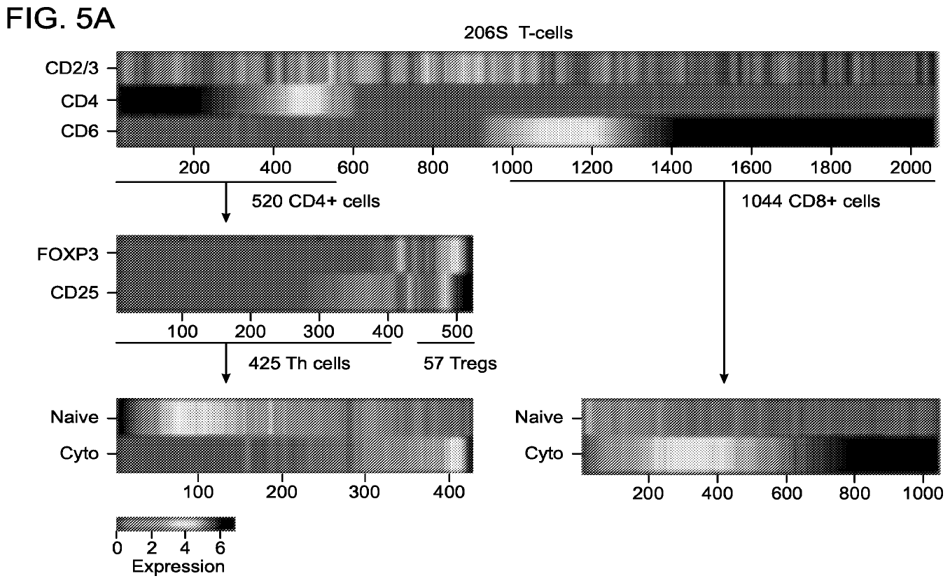

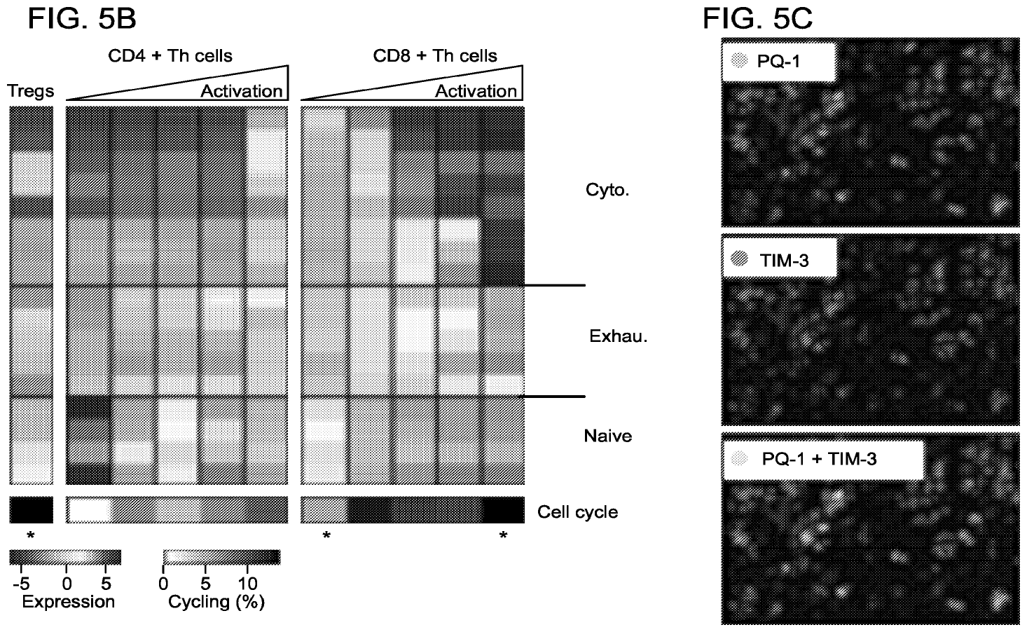

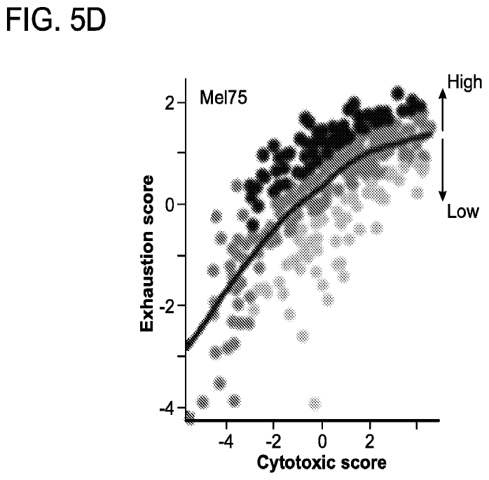

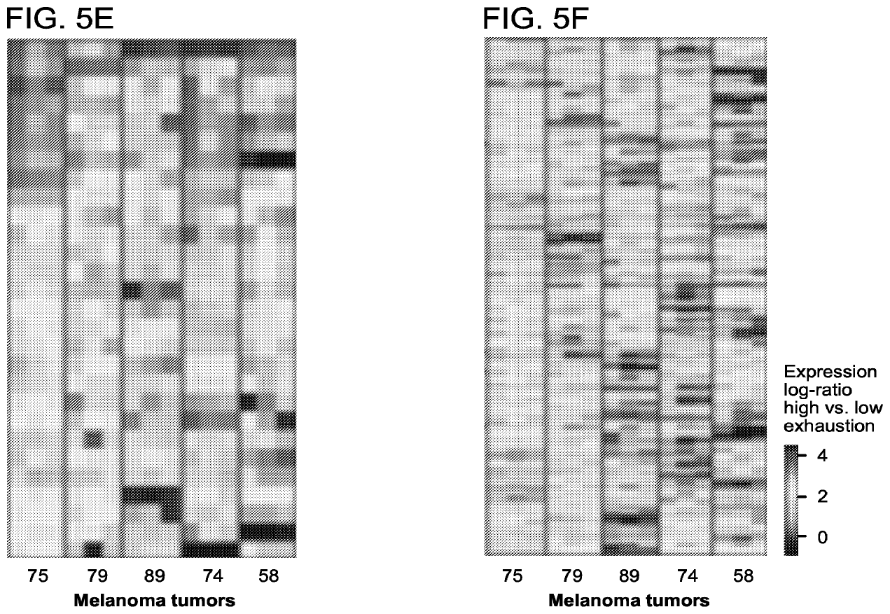

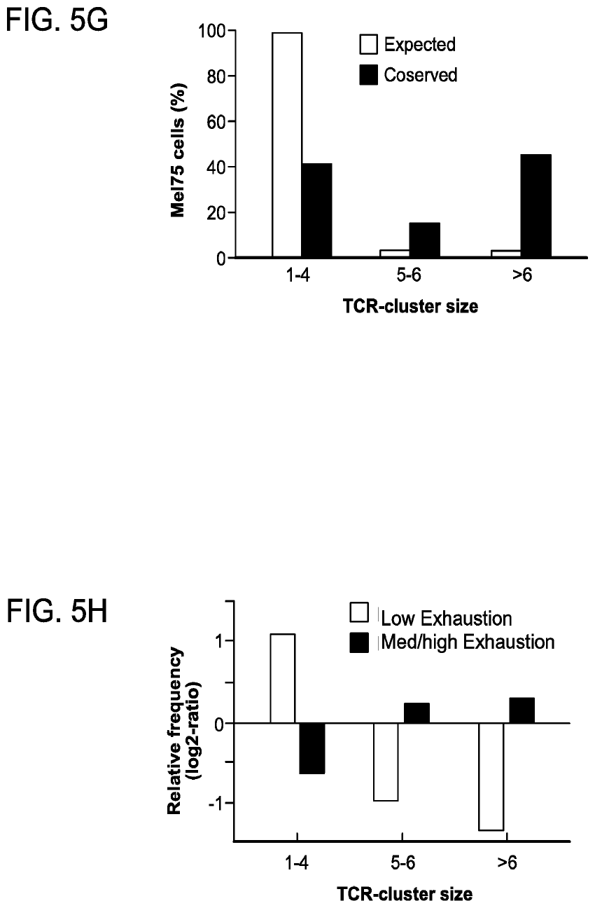

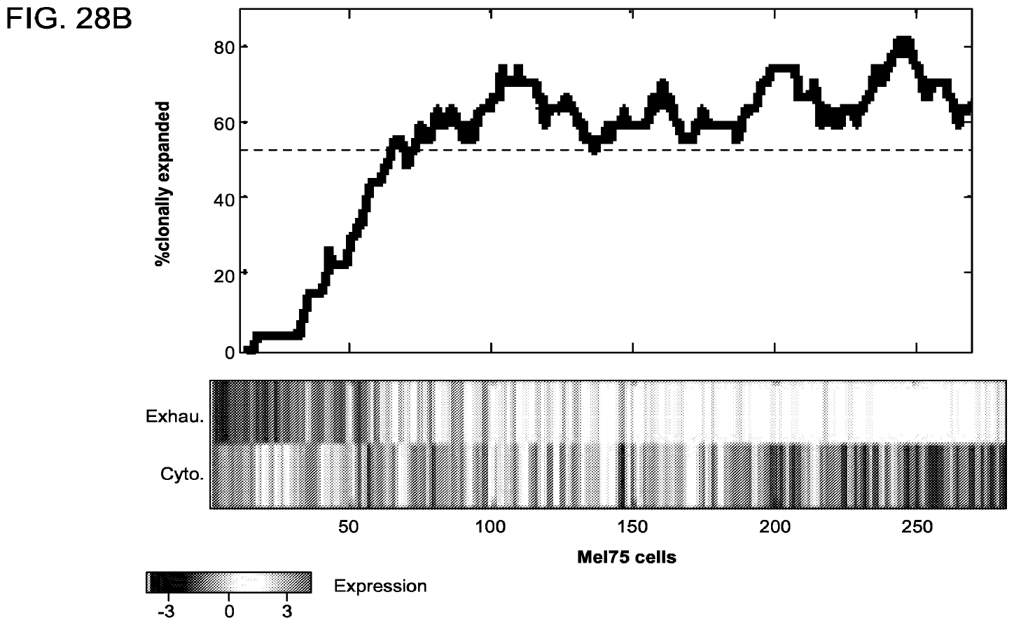

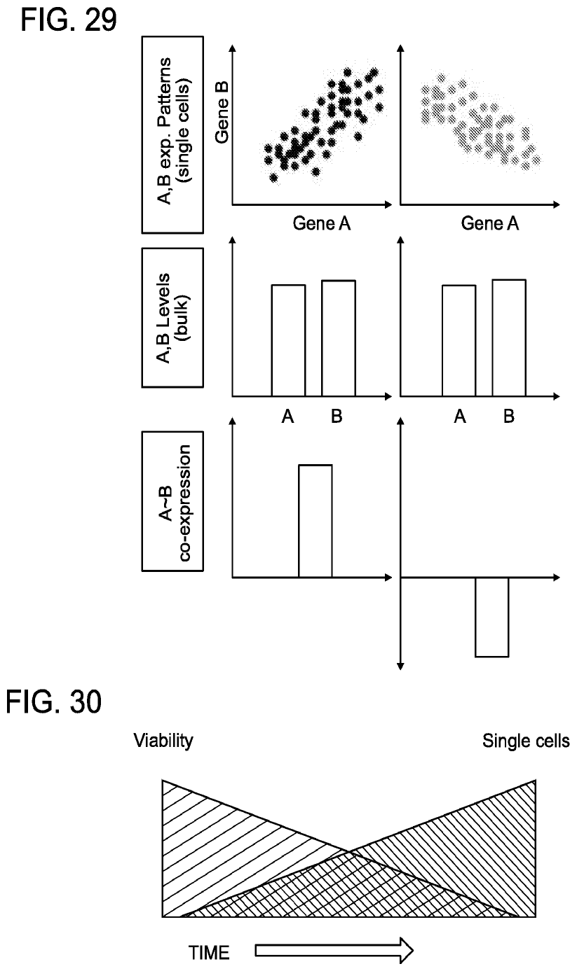

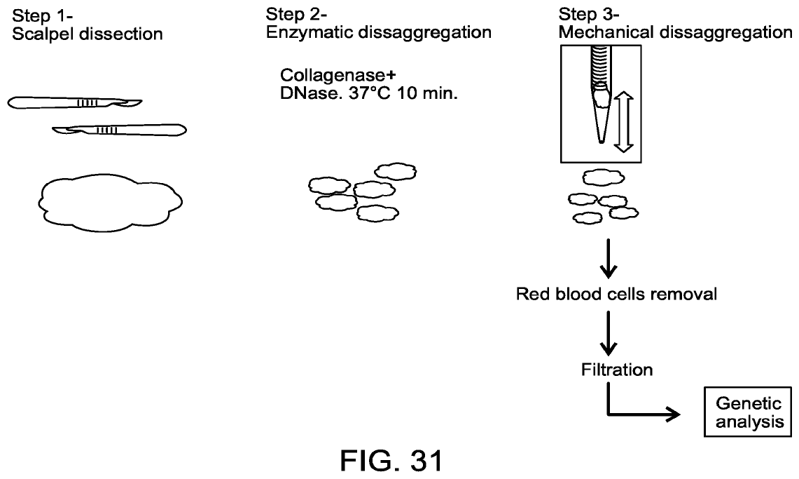

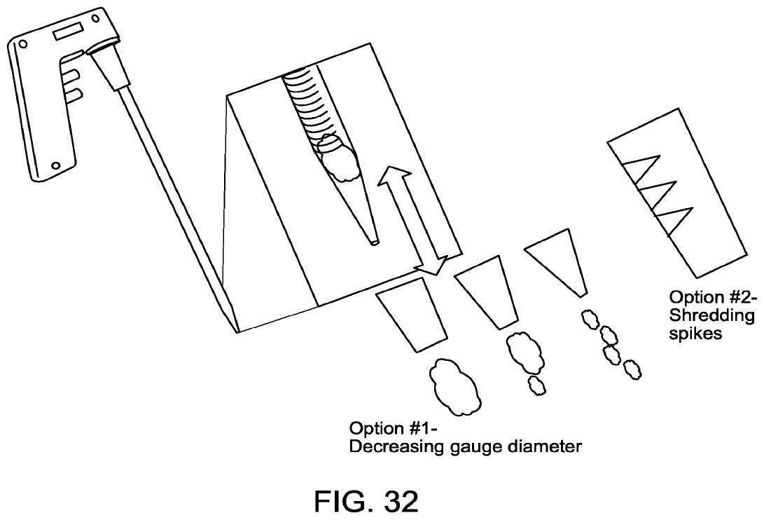

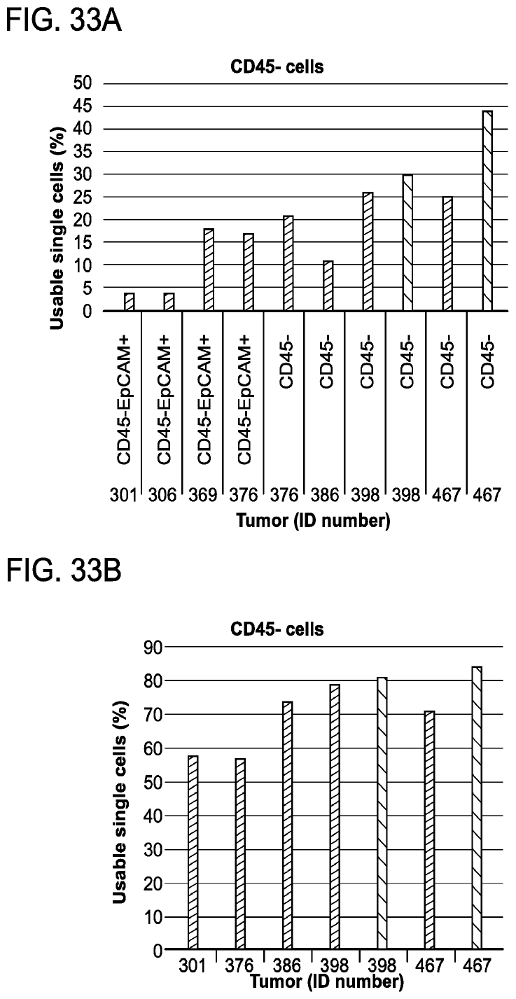

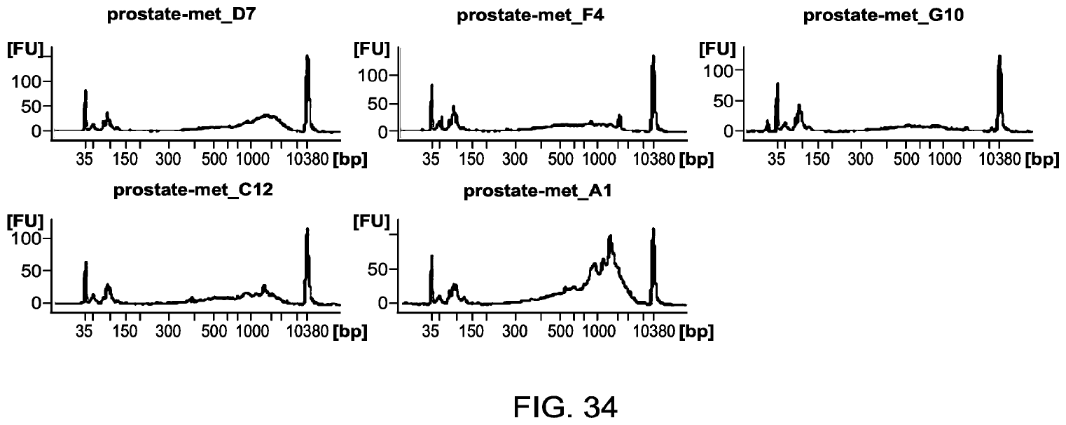

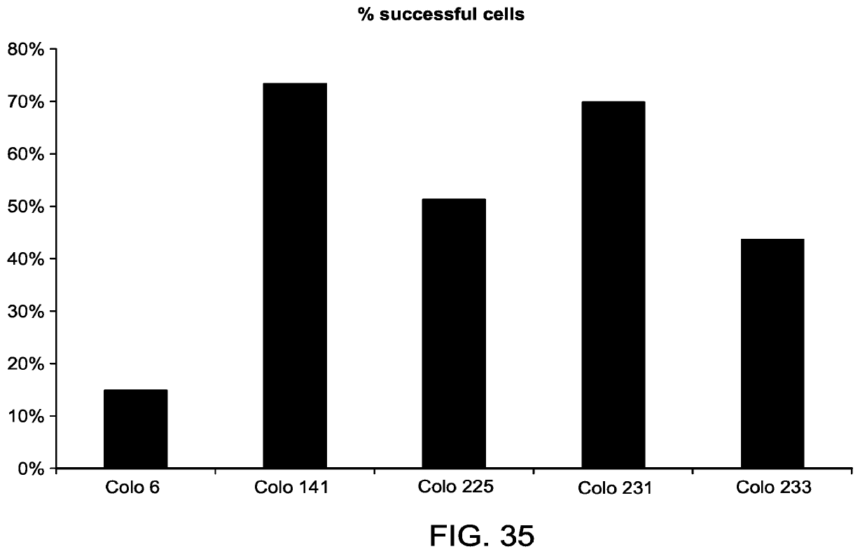

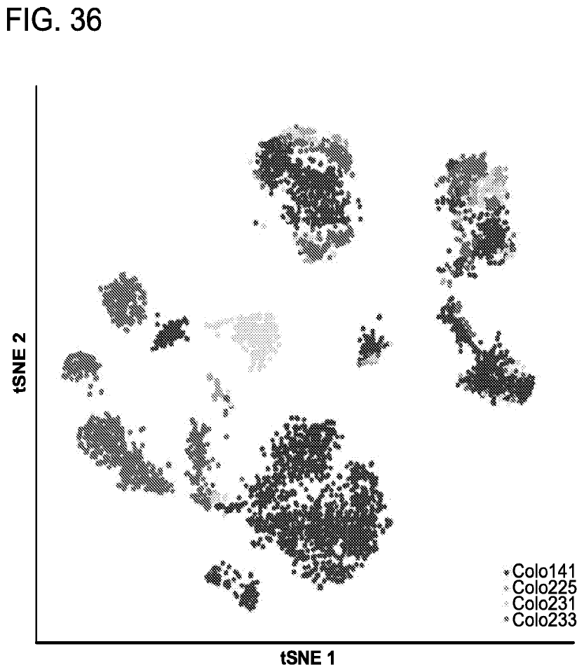

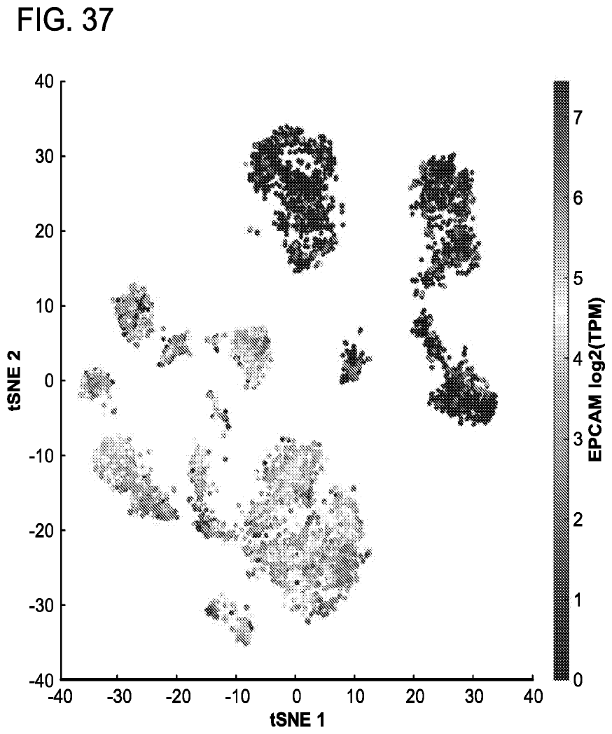

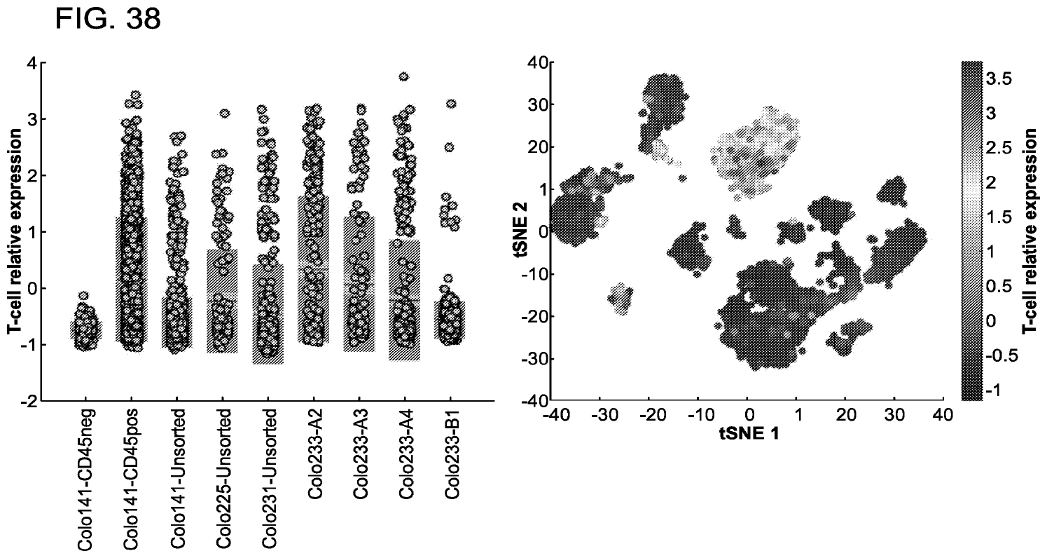

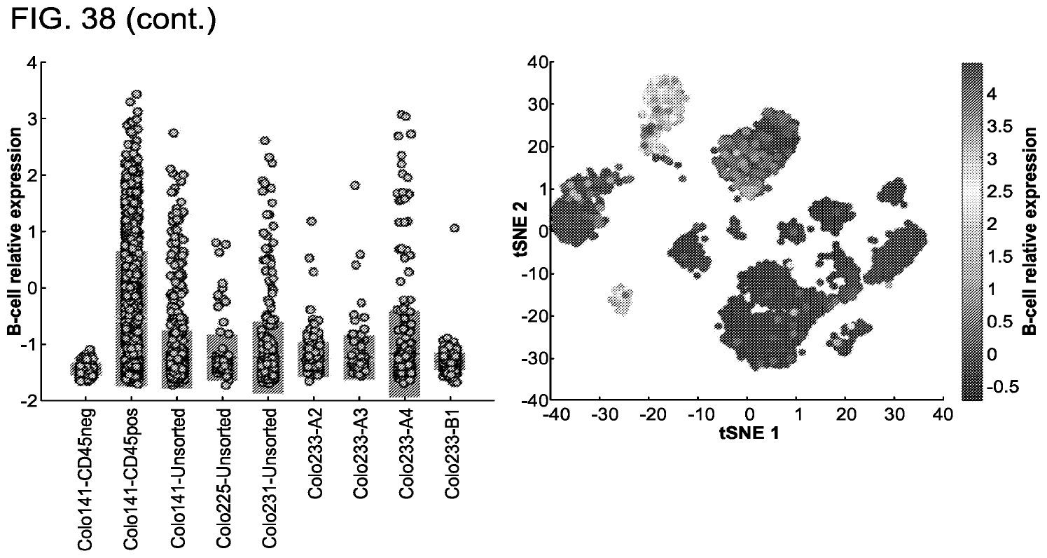

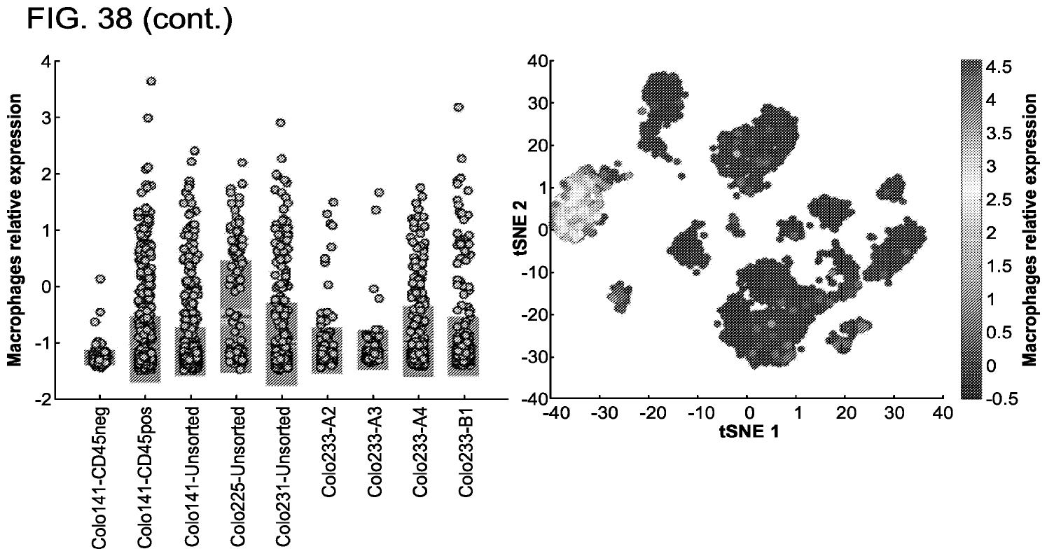

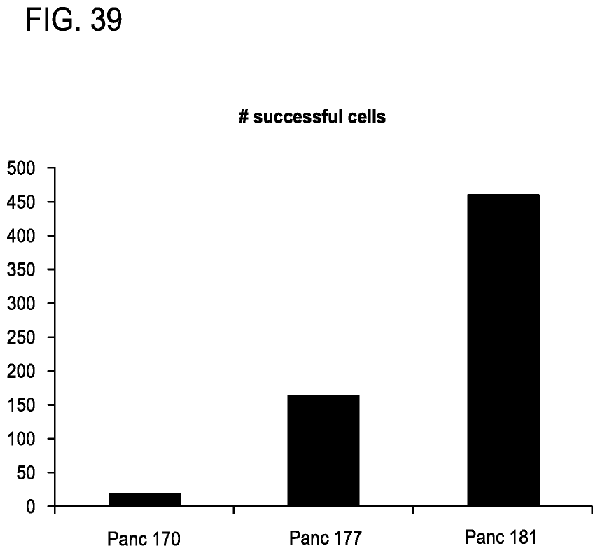

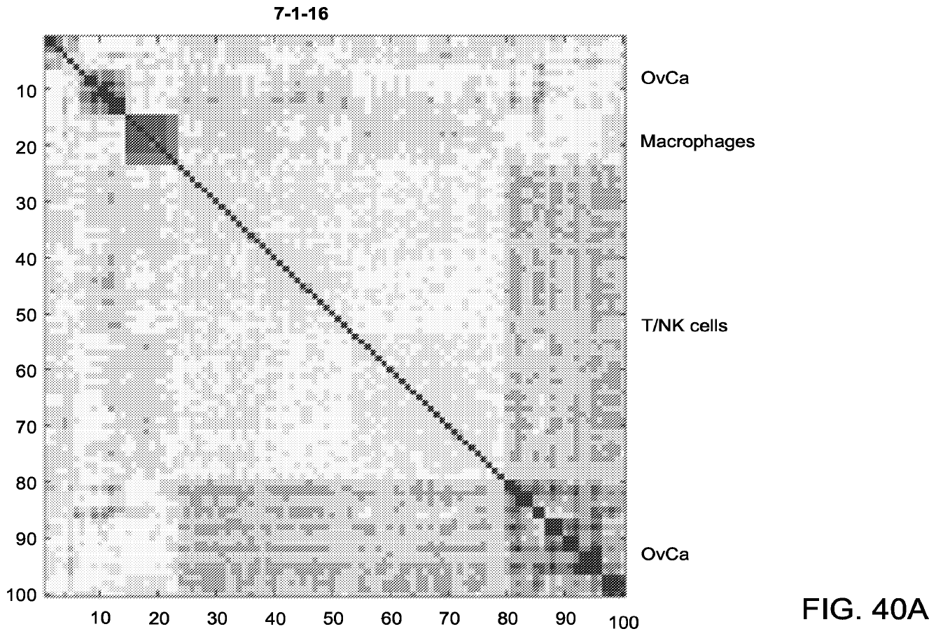

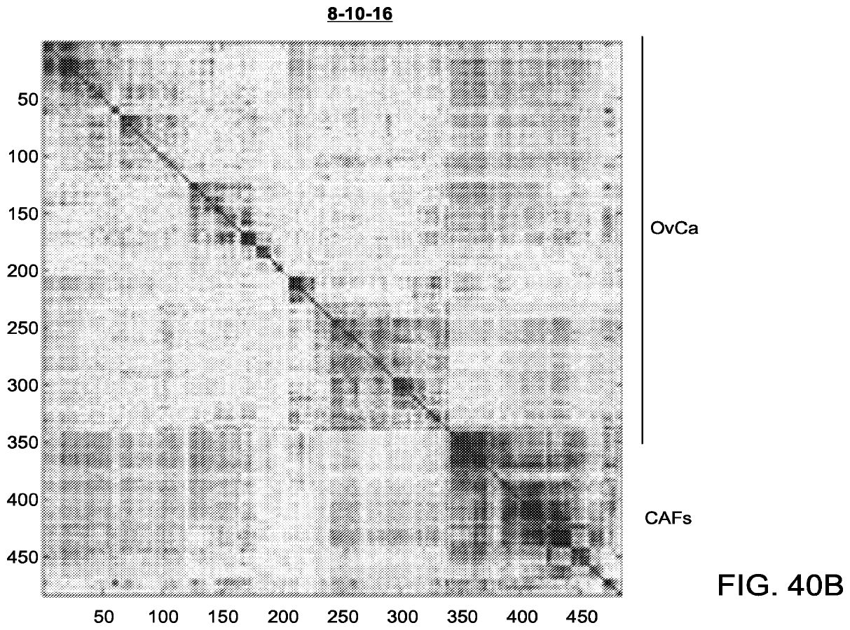

P(i,j)=P_{βv}(i,j)/*P_{βj}(i,j)*P_{αv}(i,j)*P_{αj}(i,j)P(i,j) P_{βv}(i,j)/*P_{βj}(i,j)*P_{αv}(i,j)*P_{αj}(i,j)P(i,j). For each segment, the probability equals one if segment usage is unresolved in at least one of the cells of the pair, and otherwise (i.e., if the two cells have the same allele) the probability is 1/N, where N is the number of distinct alleles that were identified for that segment. The TCR usage of one exemplary cluster is indicated. FIG. 28B is a graph wherein Mel75 cells were ordered by the average relative expression of Exhaustion and Cytotoxic genes, as shown in FIG. 5B, and the percentage of clonally expanded cells (i.e., belonging to the clusters indicated in FIG. 28A) is shown with a moving average of 20 cells, demonstrating the depletion of expanded T cells among cells with high cytotoxic and low exhaustion expression. Dashed line indicates the overall frequency of clonally expanded cells. Note that the top and bottom panels are aligned but that due to the use of a 20-cell moving average, the top panel can only start at the 11^{th} cell and end at the 11^{th} cell from the end.

FIG. 29 is a series of graphs showing the identification of distinct co-expression programs may require single cell analysis. Schematic depicting how single-cell RNA-seq can distinguish two scenarios that are indistinguishable by bulk profiling. Across individual tumor cells (top), genes A and B are either positively (left) or negatively (right) correlated. In bulk tumor (middle), the average expression of A,B cannot distinguish the two scenarios, whereas co-expression estimates from single cell RNA-seq (bottom) do so.

FIG. 30 is a graph demonstrating viability of single cells as a function of time.

FIG. 31 is a schematic diagram depicting the stepwise dissociation of tumor to produce a single cell suspension.

FIG. 32 is a schematic of mechanical disaggregation of tumor sample.

FIG. 33A and FIG. 33B is a series of bar charts that show success rates of different breast cancer biopsy samples after single cell RNA library constructions. FIG. 33A shows enriched tumor fraction analysis. FIG. 33B shows enriched immune cell fraction analysis. Blue columns indicate “good cells”-cutoff of 2K detected genes per single cell, while orange columns represent “useable cells”-cutoff of 1K detected genes per single cell.

FIG. 34 is a series of graphs showing whole transcriptome amplification of RNA from single cells. FIG. 34 shows a quality control description of 4 single cells and the minor amplification of cDNA fragment at the 1000 bp area (x axis) as measured by Bio Analyzer (Agilent). The last graph describes 10 single cells that were processed together and yielded a larger fragment of cDNA (titled A1).

FIG. 35 is a series of graphs showing that since implementation of improved protocols, the number of cells that pass quality control measures and are classified as successful cells that are used for analysis has been substantially improved.

FIG. 36 is a graph showing a t-Distributed Stochastic Neighbor Embedding (t-SNE) analysis, which is a dimensionality reduction tool that plots high-dimensional data (i.e., transcriptomes) in a two-dimensional space. Each dot represents a single-cell transcriptomes derived from colon cancer patients. The proximity of each cell to each other indicates their similarity in transcriptional space, i.e., the closer cells are located to each other, the more they resemble each other. This data indicates that there is substantial heterogeneity of tumor cells derived from different patients. On the other hand, non-malignant cells (for example immune cells) group irrespective of their tumor of origin, indicating a greater similarity across patients.

FIG. 37 is a graph of a tSNE analysis showing a clear separation of tumor cells (highlighted in red) from non-malignant cells (blue). The plot also indicates inter-tumor heterogeneity and variable expression of tumor markers (i.e., different levels of expression of tumor specific EPCAM).

FIG. 38 (left) are plots that indicate the expression of immune cell specific genes sets, which uniquely identify cells as T cells, B cells and macrophages, the three major immune cells infiltrating cancer. FIG. 38 (right) shows the expression of the respective marker in individual cells across patients highlighted in red. Red color indicates high expression of the respective program. Notably, immune cells from different patients cluster together in tSNE, indicating their similarity across patients.

FIG. 39 is a bar chart that indicates the number of cells that were successfully sequenced on Seq-Well, a novel single-cell sequencing platform. This plot highlights the improved number of successful cells with improved dissociation protocols. Furthermore, it indicates that the used protocols provide viable and high quality cells for different single-cell sequencing platforms.

FIG. 40A-FIG. 40B is a series of charts showing two heat-maps illustrating the hierarchical clustering results of single-cells, which were isolated from the same patient at two distinct time points. As shown in FIG. 40A, the first sample (procured on Jul. 1, 2016) yielded only 100 cells with sufficient transcriptome quality. About 40 of these cells were actual cancer cells, while the other cells were cells of the tumor microenvironment. In comparison, as shown in FIG. 40B, a sample procured from the same patient about one month later (Aug. 10, 2016) and processed with improved protocols as set forth in Example 8 yielded a substantially higher number of cells (˜475 cells). Notably, the number and the proportion of actual cancer cells was significantly improved, accounting for 350 cells or ˜75% of the entire sampled population, respectively. It is also important to note that both specimens were sequenced on the same platform (10×) and thereby reflect an important comparison highlighting protocol improvements achieved between the procurement of these samples.

### DETAILED DESCRIPTION OF THE INVENTION

Tumors are complex ecosystems defined by spatiotemporal interactions between heterogeneous cell types, including malignant, immune, and stromal cells (D. Hanahan and R. A. Weinberg, 2011 Cell, 144: 646-674). Each tumor's cellular composition, as well as the interplay between these components, may exert critical roles in cancer development (C. E. Meacham and S. J. Morrison, 2013 Nature, 501: 328-337). However, prior to the invention described herein, the specific components, their salient biological functions, and the means by which they collectively define tumor behavior were incompletely characterized.

Tumor cellular diversity poses both challenges and opportunities for cancer therapy. This is most clearly demonstrated by the remarkable but varied clinical efficacy achieved in malignant melanoma with targeted therapies and immunotherapies. First, immune checkpoint inhibitors produce substantial clinical responses in some patients with metastatic melanomas (Hodi et al., 2010 N. Engl. J. Med., 363: 711-723; Brahmer et al., 2010 J. Clin. Oncol. Off. J. Am. Soc. Clin. Oncol., 28: 3167-3175; Brahmer et al., 2012 N. Engl. J. Med., 366: 2455-2465; Topalian et al., 2012 N. Engl. J. Med., 366: 2443-2454; and Hamid et al., 2013 N. Engl. J. Med., 369: 134-144); however, prior to the invention described herein, the genomic and molecular determinants of response to these agents was poorly understood. Although tumor neoantigens and PD-L1 expression clearly contribute (Weber et al., 2013 J. Clin. Oncol. Off. J. Am. Soc. Clin. Oncol., 31: 4311-4318; K. M. Mahoney and M. B. Atkins, 2014 Oncol. Williston Park N. 28, Suppl 3, 39-48; Larkin et al., 2015 N. Engl. J. Med., 373: 23-34), it is likely that other factors from subsets of malignant cells, the microenvironment, and tumor-infiltrating lymphocytes (TILs) also play essential roles (Snyder et al., 2014 N. Engl. J. Med., 371: 2189-2199). Second, melanomas that harbor the BRAFV600E mutation are commonly treated with RAF/MEK-inhibition prior to or following immune checkpoint inhibition. Although this regimen improves survival, virtually all patients eventually develop resistance to these drugs (Wagle et al., 2011 J. Clin. Oncol. doi:10.1200/JCO.2010.33.2312; Van Allen et al., 2014 Cancer Discov, 4: 94-109). Unfortunately, no targeted therapy currently exists for patients whose tumors lack BRAF mutations—including NRAS mutant tumors, those with inactivating NF1 mutations, or rarer events (e.g., RAF fusions). Collectively, these factors highlight the need for a deeper understanding of melanoma composition and its impact on clinical course.

The next wave of therapeutic advances in cancer will likely be accelerated by emerging technologies that systematically assess the malignant, microenvironmental, and immunologic states most likely to inform treatment response and resistance. An ideal approach would assess salient cellular heterogeneity by quantifying variation in oncogenic signaling pathways, drug-resistant tumor cell subsets, and the spectrum of immune, stromal and other cell states that may inform immunotherapy response. Toward this end, emerging single-cell genomic approaches enable detailed evaluation of genetic and transcriptional features present in 100 s-1000 s of individual cells per tumor (Shalek et al., 2013 Nature, 498: 236-240; Patel et al., 2014 Science, 344: 1396-1401; Macosko et al., 2015 Cell, 161: 1202-1214). In principle, this approach might provide a comprehensive means to identify all major cellular components simultaneously, determine their individual genomic and molecular states (Patel et al., 2014 Science, 344: 1396-1401), and ascertain which of these features may predict or explain clinical responses to anticancer agents.

As described in detail below, single-cell RNA-seq was utilized to examine intra- and inter-tumoral heterogeneities in both malignant and non-malignant cell types and states, their drivers and interrelationships in the complex tumor cellular ecosystem. Specifically, single-cell RNA-seq was used to characterize 4,645 malignant and non-malignant cells of the tumor microenvironment from 19 patient-derived melanomas. The analysis uncovered intra- and inter-individual, spatial, functional and genomic heterogeneity in melanoma cells and associated tumor components that shape the microenvironment, including immune cells, CAFs, and endothelial cells. A cell state was identified in a subpopulation of all melanomas studied that is linked to resistance to targeted therapies and the presence of a dormant drug-resistant population in a number of melanoma cell lines was validated using different approaches.

By leveraging single cell profiles from a few tumors to deconvolve a large collection of bulk profiles from TCGA, different microenvironments that are associated with distinct malignant cell profiles were identified, as well as a subset of genes expressed by one cell type (e.g., CAFs) that may influence the proportion of cells present of another cell type (e.g., T cells), suggesting the importance of intercellular communication for tumor phenotype. Putative interactions between stromal-derived factors and the immune-cell abundance were validated in a large independent set of melanoma core biopsies. These observations suggest that new diagnostic and therapeutic strategies that consider tumor cell composition rather than bulk expression may prove advantageous. Finally, putative functional differences between exhausted and cytotoxic T cells were identified—only detectable in the co-variation of the expression of several transcripts directly measurable by single cell RNA-seq—which serve as biomarkers for immunotherapies, such as immune checkpoint inhibitors.

As described herein, the interplay between these cell types and functional states in space and time is clarified and the ability to carry out numerous, highly-multiplexed single cell observations within a tumor provides unprecedented power for identifying meaningful cell subpopulations and gene expression programs that can inform both the analysis of bulk transcriptional data and precision treatment strategies. Single cell genomic profiling enables a deeper understanding of the complex interplay among cells within the tumor ecosystem and its evolution in response to treatment, thereby providing a versatile new tool for future translational applications.

### Dissociation of Human Tumor to Single Cell Suspension

Dissociation of patient-derived tumor is a necessary process that allows analysis in a single cell manner. Commercial reagents and academic protocols offer single-cell analysis; however, prior to the invention described herein, existing methods utilized harsh enzymes at close to room temperature for an extended period of time, which resulted in reduced single cell viability and unwanted changes to the genetic profile. For example, prior to the invention described herein, tumors from human samples were dissociated using trypsin, which reduces viability of cells and abolishes surface markers including those that are necessary for genetic analysis. Other protocols utilized prior to the invention described herein used other enzymes besides trypsin, but for longer periods of time, which was undesirable. For example, some methods utilized prior to the invention described herein took a few hours, e.g., at least 3 hours, at least 4 hours, at least 5 hours, at least 6 hours, at least 7 hours, at least 8 hours, at least 9 hours, at least 10 hours, at least 11 hours or at least 12 hours to produce a single cell suspension. As shown in FIG. 30, viability of single cells decreases over time. As such, a long preparation time is undesirable.

The combined mechanical and biochemical treatments of the methods described herein provide unexpected advantages over the current methods in the art by allowing for a rapid and efficient process for generating single cell suspensions for analytical and diagnostic use. The method described herein is short (e.g., about one hour, which is significantly faster than existing methods which take between 5 and 12 hours), effective at generating a high percentage of viable cells (e.g., at least about 60%), and applicable to several tumors and biopsy types. The methods described herein also limit the amount of time the cells are exposed to 37° C., which ensures high RNA quality. Additionally, unlike existing methods, which alter the cell surface markers, and the cellular genetic profile, the methods described herein provide single cell suspensions with authentic genetic profiles, which allows for accurate genetic analysis. Specifically, described herein is the design and application of a strategy to dissociate human tumor into single cells, which are subsequently analyzed via, e.g., single-cell RNA sequencing or a chemotherapy sensitivity test.

The methods described herein are useful in processing different types of tumors, e.g., at least melanoma, ovarian cancer, breast cancer, colorectal cancer, pancreatic cancer, lung cancer, head and neck cancer, and prostate cancer. The methods described herein are also useful for processing different tissue types, e.g., at least solid tumors, core needle biopsies, fine needle aspiration samples, malignant effusion samples (peritoneal, pleural and pericardial effusion), bone marrow aspirates, aspirates from bone biopsy of a metastatic prostate lesion, and blood samples. The methods described herein offer the ability to process malignant tissue from different species, e.g., at least human material and material from animal models or patient-derived xerographs (PDXs) such as rodents, e.g., mice. Various compositions of cells, e.g., solid tissue, bone, spheroids (clusters of cells from malignant effusions or cultured cells), and single cell solutions (including malignant effusions), are also processed using the methods described herein. The methods described herein are applicable to malignant and non-malignant tissue derived from patients, e.g., cancer tissue, non-cancerous tissue, but otherwise diseased tissue (e.g., lymph nodes from a patient with an inflammatory disorder, skin, bone, etc.), and healthy tissue (e.g., skin, lymph nodes, and other organs). The methods described herein offer the ability to isolate different cell types including malignant and non-malignant cells, e.g., tumor cells, immune cells (e.g., T-cells, B-cells, NK-cells, macrophages, and dendritic cells), cancer-associated fibroblasts, endothelial cells, and other cells that are sensitive to enzymatic digestion.

FIG. 31 presents a schematic diagram depicting the stepwise dissociation of tumor to produce a single cell suspension. First, the tumor is dissected/cut into small tissue pieces (e.g., <1 mm³) using, e.g., a scalpel to dissect the tumor sample. Next, the tumor sample is subject to enzymatic degradation, i.e., collagenase and DNase at 37° C. for 10 minutes. This relatively short time frame of enzymatic disaggregation allows for increased viability of single cells. Finally, mechanical disaggregation of the tumor sample is performed, wherein the tumor sample is repeatedly passed through a pipette tip, e.g., a plastic pipette tip, as shown in step 3 of FIG. 31.

As shown in FIG. 32, the inner diameter of a pipette tip is utilized for separation of tumor tissue and progressively smaller pipette tips are used to dissociate the tumor sample into single cells, e.g., 25 ml pipette tips, 10 ml pipette tips, 5 ml pipette tips, and 1 ml pipette tips are used to dissociate the tumor sample. In some cases, the pipette gauge diameter is progressively decreased for better disaggregation of tissue. In this manner, the tissue size is reduced to a single cell suspension. Optionally, the tumor sample stays within the same pipette tube, i.e., only the pipette tip itself is exchanged for one with a smaller diameter as an “add-on device” or “adapter” (FIG. 32). In this matter, cell loss is reduced. Alternatively (or in addition), shredding spikes (i.e., teeth) are placed within a pipette tip for better disaggregation of tissue (FIG. 32). Subsequently, red blood cells are removed, the sample is filtered, and single cells are isolated.

An exemplary method utilized in the Examples below is as follows. Resected tumors were transported in DMEM (ThermoFisher Scientific) on ice immediately after surgical procurement. Tumors were rinsed with phosphate-buffered saline (PBS; Life Technologies). A small fragment was stored in RNA-protect (Qiagen) for bulk RNA and DNA isolation. Using scalpels, the remainder of the tumor was minced into tiny cubes <1 mm³ and transferred into a 50 ml conical tube (BD Falcon) containing 10 ml pre-warmed M199-media (ThermoFisher Scientific), 2 mg/ml collagenase P (Roche) and 10 U/μl DNase I (Roche). Tumor pieces were digested in this digestion media for 10 minutes at 37° C., then vortexed for 10 seconds and pipetted up and down for 1 minute using pipettes of descending sizes (25 ml, 10 ml and 5 ml). If needed, this was repeated twice more until a single-cell suspension was obtained. This suspension was then filtered using a 70 μm nylon mesh (ThermoFisher Scientific) and residual cell clumps were discarded. The suspension was supplemented with 30 ml PBS (Life Technologies) with 2% fetal calf serum (FCS) (Gemini Bioproducts) and immediately placed on ice. After centrifuging at 580 g at 4° C. for 6 minutes, the supernatant was discarded and the cell pellet was re-suspended in PBS with FCS and placed on ice prior to staining for fluorescence-activated cell sorting (FACS). In another example, for some samples, e.g., bone samples, the enzyme concentrations are doubled (e.g., about 4 mg/ml collagenase P (Roche) and 20 U/μl DNase I (Roche)), and a vortex is utilized to shake the sample for 10 seconds every 2 minutes during the 10 minute incubation step at 37° C.

### Single-Cell RNA-Seq

Tumors are multicellular assemblies that encompass cells with distinct genotypic and phenotypic states. As described herein, single-cell RNA-seq was applied to thousands of malignant and non-malignant cells derived from metastatic melanomas to examine tumor ecosystems. Specifically, single-cell RNA-seq was utilized to examine 4,645 single cells isolated from 19 patient melanomas, profiling malignant, immune, stromal and endothelial cells. As described in detail below, malignant cells within the same tumor displayed transcriptional heterogeneity associated with the cell cycle, spatial context, and a drug resistance program. In particular, all tumors harbored malignant cells from two distinct transcriptional cell states, such that “MITF-high” tumors also contained “AXL-high” tumor cells, which are often intrinsically resistant to RAF/MEK inhibition. The proportion of AXL-high cells increased in tumors and cell lines following treatment with RAF/MEK inhibition. As described herein, single-cell analyses also suggested distinct tumor microenvironmental patterns, including cell-to-cell interactions between stromal, immune and malignant cells that were independently supported by a large melanoma core biopsy collection. Finally, analysis of tumor-infiltrating T cells revealed exhaustion programs, their connection to T cell activation and to clonal expansion, and their variability across patients. Overall, the analysis described in detail below unravels the cellular ecosystem of tumors and shows that single cell genomics offers new insights with implications for both targeted and immune therapies.

### Kits

The present compositions may be assembled into kits for use in disaggregating a tissue sample into single cells. Kits according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Materials and Methods

### Tissue Handling and Tumor Disaggregation

Resected tumors were transported in DMEM (ThermoFisher Scientific) on ice immediately after surgical procurement. Tumors were rinsed with PBS (Life Technologies). A small fragment was stored in RNA-protect (Qiagen) for bulk RNA and DNA isolation. Using scalpels, the remainder of the tumor was minced into tiny cubes <1 mm³ and transferred into a 50 ml conical tube (BD Falcon) containing 10 ml pre-warmed M199-media (ThermoFisher Scientific), 2 mg/ml collagenase P (Roche) and 10 U/μl DNase I (Roche). Tumor pieces were digested in this digestion media for 10 minutes at 37° C., then vortexed for 10 seconds and pipetted up and down for 1 minute using pipettes of descending sizes (25 ml, 10 ml and 5 ml). If needed, this was repeated twice more until a single-cell suspension was obtained. This suspension was then filtered using a 70 μm nylon mesh (ThermoFisher Scientific) and residual cell clumps were discarded. The suspension was supplemented with 30 ml PBS (Life Technologies) with 2% fetal calf serum (FCS) (Gemini Bioproducts) and immediately placed on ice. After centrifuging at 580 g at 4° C. for 6 minutes, the supernatant was discarded and the cell pellet was re-suspended in PBS with FCS and placed on ice prior to staining for FACS.

### FACS

Single-cell suspensions were stained with CD45-fluorescein isothiocyanate (FITC) (VWR) and Calcein-AM (Life Technologies) per manufacturer recommendations. For sorting of ex vivo co-cultured cancer-associated fibroblasts, a CD9O-PE antibody (BioLegend) was used. First, doublets were excluded based on forward and sideward scatter, then viable cells (Calcein-high) were gated and single cells were sorted (CD45+ or CD45− or CD45− CD90+) into 96-well plates chilled to 4° C. pre-prepared with 10 μl TCL buffer (Qiagen) supplemented with 1% beta-mercaptoethanol (lysis buffer). Single-cell lysates were sealed, vortexed, spun down at 3700 rpm at 4° C. for 2 minutes, immediately placed on dry ice and transferred for storage at −80° C.

RNA and DNA Isolation from Bulk Specimens

RNA and DNA were isolated using the Qiagen minikit following the manufacturer's recommendations.

### Whole Transcriptome Amplification

Whole Transcriptome amplification (WTA) was performed with a modified SMART-Seq2 protocol, as described previously (Picelli et al., 2013 Nat. Methods., 10: 1096-1098; Trombetta et al., Preparation of Single-Cell RNA-Seq Libraries for Next Generation Sequencing. Curr. Protoc. Mol. Biol. Ed. Frederick M Ausubel Al. 107, 4.22.1-4.22.17 (2014)), with Maxima Reverse Transcriptase (Life Technologies) used in place of Superscript II.

### Library Preparation and RNA-Seq

WTA products were cleaned with Agencourt XP DNA beads and 70% ethanol (Beckman Coulter) and Illumina sequencing libraries were prepared using Nextera XT (Illumina), as previously described (Trombetta et al., Preparation of Single-Cell RNA-Seq Libraries for Next Generation Sequencing. Curr. Protoc. Mol. Biol. Ed. Frederick M Ausubel Al. 107, 4.22.1-4.22.17 (2014)). The 96 samples of a multiwall plate were pooled together, and cleaned with two 0.8×DNA SPRIs (Beckman Coulter). Library quality was assessed with a high sensitivity DNA chip (Agilent) and quantified with a high sensitivity dsDNA Quant Kit (Life Technologies). Samples were sequenced on an Illumina NextSeq 500 instrument using 30 bp paired-end reads.

### Whole-Exome Sequencing and Analysis

Exome sequences were captured using Illumina technology and Exome sequence data processing and analysis were performed using the Picard and Firehose pipelines at the Broad Institute. The Picard pipeline (picard.sourceforge.net) was used to produce a BAM file with aligned reads. This includes alignment to the hg19 human reference sequence using the Burrows-Wheeler transform algorithm (H. Li and R. Durbin, 2009 Bioinforma. Oxf. Engl., 25: 1754-1760) and estimation of base quality score and recalibration with the Genome Analysis Toolkit (GATK) (broadinstitute.org/gatk/)(McKenna et al., 2010 Genome Res., 20: 1297-1303). All sample pairs passed the Firehose pipeline including a QC pipeline to test for any tumor/normal and inter-individual contamination as previously described (Berger et al., 2011 Nature, 470: 214-20; Cibulskis et al., 2013 Nat. Biotechnol., 31: 213-9). The MuTect algorithm was used to identify somatic mutations (Cibulskis et al., 2013 Nat. Biotechnol., 31: 213-9). MuTect identifies candidate somatic mutations by Bayesian statistical analysis of bases and their qualities in the tumor and normal BAMs at a given genomic locus. To reduce false positive calls reads covering sites of an identified somatic mutation were additionally analyzed and realigned with NovoAlign (novocraft.com) and performed additional iteration of MuTect inference on newly aligned BAM files. Furthermore, somatic mutation cells were filtered using a panel of over 8,000 TCGA Normal samples. Small somatic insertions and deletions were detected using the Strelka algorithm (Saunders et al., 2012 Bioinforma. Oxf. Engl., 28: 1811-7) and similarly subjected to filtering out potential false positive using the panel of TCGA Normal samples. Somatic mutations including single-nucleotide variants, insertions, and deletions were annotated using Oncotator (Ramos et al., 2015 Hum. Mutat., 36: E2423-9). Copy-ratios for each captured exon were calculated by comparing the mean exon coverage with expected coverage based on a panel of normal samples. The resulting copy ratio profiles were then segmented using the circular binary segmentation (CBS) algorithm (E. S. Venkatraman and A. B. Olshen, 2007 Bioinforma. Oxf. Engl., 23: 657-63).

### Processing of RNA-Seq Data

Following sequencing on the NextSeq, BAM files were converted to merged, demultiplexed FASTQs. Paired-end reads were then mapped to the UCSC hg19 human transcriptome using Bowtie (Langmead et. al., 2009 Genome Biol., 10: R25) with parameters “-q --phred33-quals -n 1 -e 99999999 -l 25 -I 1 -X 2000 -a -m 15 -S -p 6”, which allows alignment of sequences with single base changes such as due to point mutations. Expression levels of genes were quantified as Ei,j=log 2(TPMi,j/10+1), where TPMi,j refers to transcript-per-million (TPM) for gene i in sample j, as calculated by RSEM (60) v1.2.3 in paired-end mode. TPM values were divided by 10 since the complexity of the single cell libraries was estimate to be on the order of 100,000 transcripts and would like to avoid counting each transcript ˜10 times, as would be the case with TPM, which may inflate the difference between the expression level of a gene in cells in which the gene is detected and those in which it is not detected. When evaluating the average expression of a population of cells by pooling data across cells (e.g., all cells from a given tumor or cell type) the division by 10 was not required and the average expression was defined Ep(I)=log 2(TPM(I)+1), where I is a set of cells.

For each cell, the number of genes for which at least one read was mapped was quantified, and the average expression level of a curated list of housekeeping genes (Table 14). All cells with either fewer than 1,700 detected genes or an average housekeeping expression (E, as defined above) below 3 were excluded. For the remaining cells, the pooled expression of each gene was calculated as (Ep), and genes with an aggregate expression below 4 were excluded, which defined a different set of genes in different analyses depending on the subset of cells included. For the remaining cells and genes, relative expression was defined by centering the expression levels, Eri,j=Ei,j−average [Ei,1 . . . n].

TABLE 14
Curated list of housekeeping genes used for quality control analysis

| | | |
|---|---|---|
| ACTB | RPL37 | RPS4X |
| B2M | RPL38 | RPS5 |
| HNRPLL | RPL39 | RPS6 |
| HPRT | RPL39L | RPS6KA1 |
| PSMB2 | RPL3L | RPS6KA2 |
| PSMB4 | RPL4 | RPS6KA3 |
| PPIA | RPL41 | RPS6KA4 |
| PRPS1 | RPL5 | RPS6KA5 |
| PRPS1L1 | RPL6 | RPS6KA6 |
| PRPS1L3 | RPL7 | RPS6KB1 |
| PRPS2 | RPL7A | RPS6KB2 |
| PRPSAP1 | RPL7L1 | RPS6KC1 |
| PRPSAP2 | RPL8 | RPS6KL1 |
| RPL10 | RPL9 | RPS7 |
| RPL10A | RPLP0 | RPS8 |
| RPL10L | RPLP1 | RPS9 |
| RPL11 | RPLP2 | RPSA |
| RPL12 | RPS10 | TRPS1 |
| RPL13 | RPS11 | UBB |
| RPL14 | RPS12 | |
| RPL15 | RPS13 | |
| RPL17 | RPS14 | |
| RPL18 | RPS15 | |
| RPL19 | RPS15A | |
| RPL21 | RPS16 | |
| RPL22 | RPS17 | |
| RPL22L1 | RPS18 | |
| RPL23 | RPS19 | |
| RPL24 | RPS20 | |
| RPL26 | RPS21 | |
| RPL27 | RPS24 | |
| RPL28 | RPS25 | |
| RPL29 | RPS26 | |
| RPL3 | RPS27 | |
| RPL30 | RPS27A | |
| RPL32 | RPS27L | |
| RPL34 | RPS28 | |
| RPL35 | RPS29 | |
| RPL36 | RPS3 | |
| | RPS3A | |

### Data Availability

Raw and processed single-cell RNA-seq data is available through the Gene Expression Omnibus (GSE72056).

### CNV Estimation

Initial CNVs (CNV0) were estimated by sorting the analyzed genes by their chromosomal location and applying a moving average to the relative expression values, with a sliding window of 100 genes within each chromosome, as previously described (Patel et al., 2014 Science, 344: 1396-1401). To avoid considerable impact of any particular gene on the moving average the relative expression values were limited to [−3,3] by replacing all values above 3 by 3, and replacing values below −3 by −3. This was performed only in the context of CNV estimation. This initial analysis is based on the average expression of genes in each cell compared to all other cells and therefore does not have a proper reference which is required to define the baseline. However, five subsets of cells that each had more limited high or low values of CNV0 and which were consistent across the genome despite the fact that these cells originate from multiple tumors were identified. These were considered as putative non-malignant cells and their CNV estimates were used to define the baseline. The normal cells included five cell types (see below, not including NK cells), which differed in gene expression patterns and accordingly also slightly in CNV estimates (e.g., the MHC region in chromosome 6 had consistently higher values in T cells than in stromal or cancer cells). Multiple baselines were defined, as the average of each cell type, and based on these the maximal (BaseMax) and minimal (BaseMin) baseline at each window of 100 genes. The final CNV estimate of cell i at position j was then defined as:

CNV f(i,j)={

CNV0(i,j)−BaseMax(j), if CNV0(i,j)>BaseMax(j)+0.2

CNV0(i,j)−BaseMin(j), if CNV0(i,j)<BaseMin(j)−0.2

0, if(j)−0.2<CNV0(i,j)<BaseMin(j)+0.2

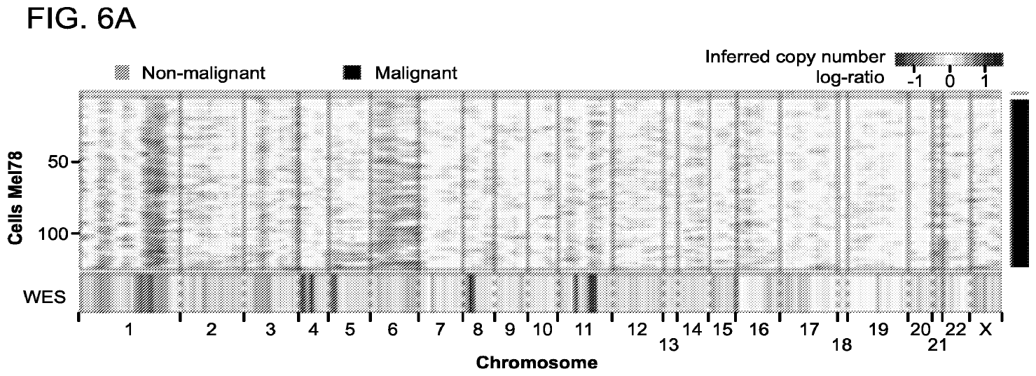

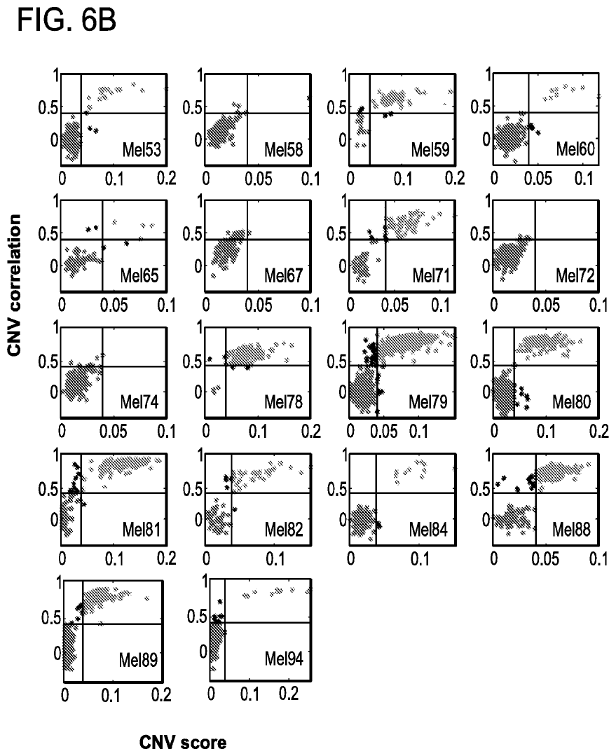

To quantitatively evaluate how likely each cell is to be a malignant or non-malignant cell the CNV pattern of each cell was summarized by two values: (1) overall CNV signal, defined as the sum of squares of the CNV f estimates across all windows; (2) the correlation of each cells' CNV f vector with the average CNV f vector of the top 10% of cells from the same tumor with respect to CNV signal (i.e., the most confidently-assigned malignant cells). These two values were used to classify cells as malignant, non-malignant, and intermediates that were excluded from further analysis, as shown in FIG. 6B.

### T-SNE Analysis and Cell Type Classification

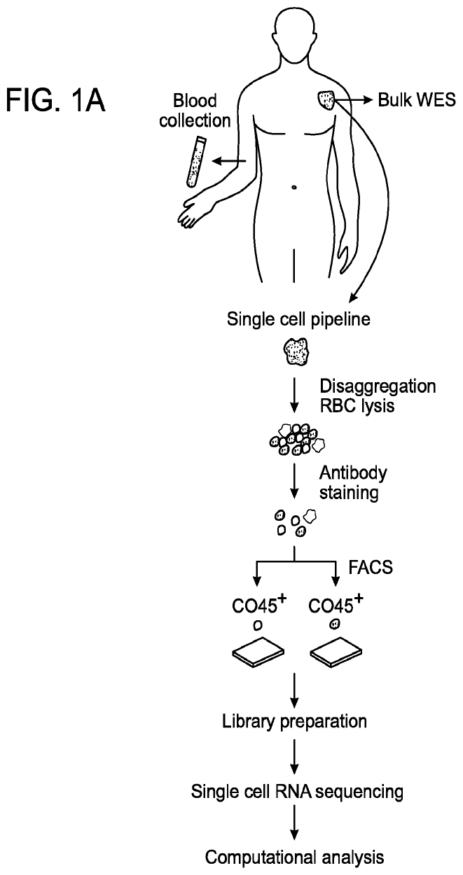

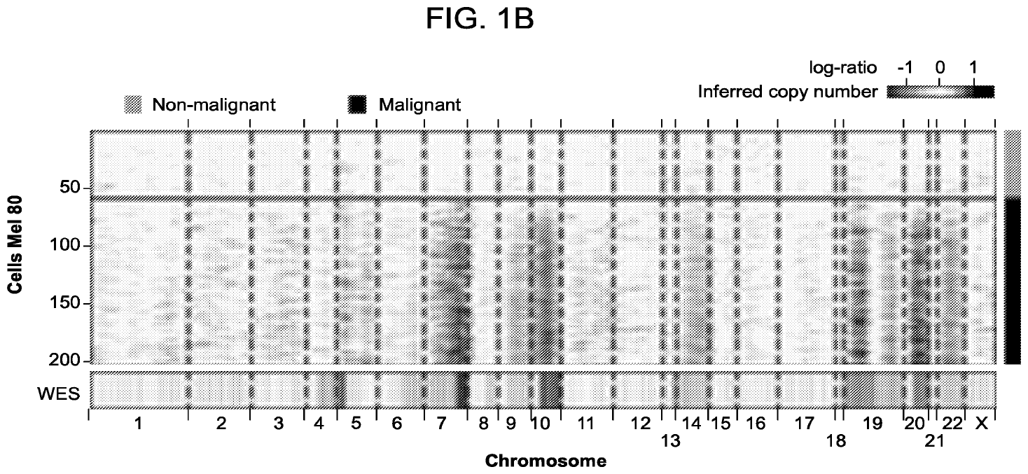

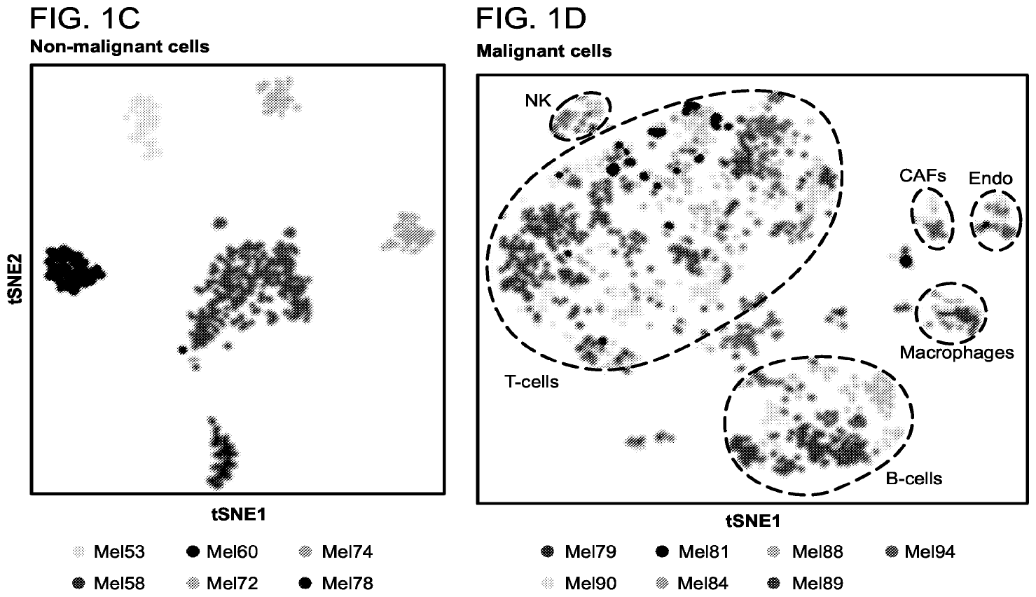

A Matlab implementation of the tSNE method was downloaded from lvdmaaten.github.io/tsne/ and applied with dim=15 to the relative expression data of malignant and to that of non-malignant cells. Since the complexity of tSNE visualization increases with the number of tumors the analysis presented in FIG. 1A-FIG. 1D was restricted to restricted to the 13 tumors with at least 100 cells, and for the malignant cell analysis the analysis was further restricted to 6 tumors with >50 malignant cells. To define cell types from the non-malignant tSNE analysis a density clustering method, DBscan (Ester, H. Kriegel, J. Sander, and X. Xu, “A density-based algorithm for discovering clusters in large spatial databases with noise,” in Proc. 2nd Int. Conf. Knowledge Discovery and Data Mining (KDD'96), 1996, pp. 226-231), was used. This process revealed six clusters for which the top preferentially expressed genes (p<0.001, permutation test) included multiple known markers of particular cell types. In this way, T cell, B-cell, macrophage, endothelial, CAF (cancer-associated fibroblast) and NK cell clusters were identified, as marked in FIG. 1D (dashed ellipses). To ensure the specificity of the assignment of individual cells to each cell type cluster, while avoiding potential doublet cells (which might be composed of two cells from distinct cell types), cells with low-quality data, and cells that spuriously cluster with a certain cell type, each non-malignant cell was scored (by CNV estimates, as described above) by the average expression of the identified cell type marker genes. Cells were classified as each cell type only if they express the marker genes for that cell type much more than those for any other cell type (average relative expression, Er, of markers for one cell type higher by at least 3 than those of other cell types, which corresponds to 8-fold expression difference). A full list of the genes preferentially expressed in each cell type as well as the subset that were used as marker genes is given in Table 3.

### Principal Component Analysis

In order to decrease the impact of inter-tumoral variability on the combined analysis of cancer cells the data within each tumor was re-centered separately, such that the average of each gene was zero among cells from each tumor. The covariance matrix used for PCA was generated using an approach outlined in (Shalek et al., 2014 Nature, 510: 363-369) to decrease the weight of less reliable “missing” values in the data. This approach aims to address the challenge that arises due to the limited sensitivity of single-cell RNA-seq, where many genes are not detected in a particular cell despite being expressed. This is particularly pronounced for genes that are more lowly expressed, and for cells that have lower library complexity (i.e., for which relatively fewer genes are detected), and results in non-random patterns in the data, whereby cells may cluster based on their complexity and genes may cluster based on their expression levels, rather than “true” co-variation. To mitigate this effect, weights are assigned to missing values, such that the weight of Ei,j is proportional to the expectation that gene i will be detected in cell j given the average expression of gene i and the total complexity (number of detected genes) of cell j.

Following PCA, the top six components were focused on, as these were the only components that both explained a significant proportion of the variance and were significantly correlated with at least one gene, where significance was determined by comparison to the top 5% (of variance explained and of top gene correlations) from 100 control PCA analyses on shuffled data. PCI had a high correlation (R=0.46) with the number of genes detected in each cell and a more specific biological function that may be associated with it was not observed. Thus, this is inferred to be a technically-driven component which is reflecting the systematic variation in the data due to the large differences in the quality and complexity of data for different cells. Subsequent analysis was focused on understanding the biological function of the next components PC2-6, which were associated with the cell cycle (PC2 and 6), regional heterogeneity (PC3) and MITF expression program (PC4 and 5).

### Cell Cycle Analysis

Previous analysis of single-cell RNA-seq in human (293T) and mouse (3T3) cell lines (Macosko et al., 2015 Cell, 161: 1202-1214), and in mouse hematopoietic stem cells (M. L. Whitfield et al., 2002 Mol. Biol. Cell., 13: 1977-2000), revealed in each case two prominent cell cycle expression programs that overlap considerably with genes that are known to function in replication and mitosis, respectively, and that have also been found to be expressed at G1/S phases and G2/M phases, respectively, in bulk samples of synchronized HeLa cells (M. L. Whitfield et al., 2002 Mol. Biol. Cell., 13: 1977-2000). Thus, a core set of 43 G1/S and 55 G2/M genes were defined that included those genes that were detected in the corresponding expression clusters in all four datasets from the three studies described above (Table 5).

TABLE 5
Core signature of cell cycle genes expressed in cycling
malignant cells from both low-cycling and high-cycling tumors.

| | |
|---|---|
| | melanoma |
| | cell cycle |
| Phase-specific genes | genes |

| | | |
|---|---|---|
| G1/S | G2/M | TYMS |
| MCM5 | HMGB2 | TK1 |
| PCNA | CDK1 | UBE2T |
| TYMS | NUSAP1 | CKS1B |
| FEN1 | UBE2C | MCM5 |
| MCM2 | BIRC5 | UBE2C |
| MCM4 | TPX2 | PCNA |
| RRM1 | TOP2A | MAD2L1 |
| UNG | NDC80 | ZWINT |
| GINS2 | CKS2 | MCM4 |
| MCM6 | NUF2 | GMNN |
| CDCA7 | CKS1B | MCM7 |
| DTL | MKI67 | NUSAP1 |
| PRIM1 | TMPO | FEN1 |
| UHRF1 | CENPF | CDK1 |
| MLF1IP | TACC3 | BIRC5 |
| HELLS | FAM64A | KIAA0101 |
| RFC2 | SMC4 | PTTG1 |
| RPA2 | CCNB2 | CENPM |
| NASP | CKAP2L | KPNA2 |
| RAD51AP1 | CKAP2 | CDC20 |
| GMNN | AURKB | GINS2 |
| WDR76 | BUB1 | ASF1B |
| SLBP | KIF11 | RRM2 |
| CCNE2 | ANP32E | MLF1IP |
| UBR7 | TUBB4B | KIF22 |
| POLD3 | GTSE1 | CDC45 |
| MSH2 | KIF20B | CDC6 |
| ATAD2 | HJURP | FANCI |
| RAD51 | HJURP | HMGB2 |
| RRM2 | CDCA3 | TUBA1B |
| CDC45 | HN1 | RRM1 |
| CDC6 | CDC20 | CDKN3 |
| EXO1 | TTK | WDR34 |
| TIPIN | CDC25C | DTL |
| DSCC1 | KIF2C | CCNB1 |
| BLM | RANGAP1 | AURKB |
| CASP8AP2 | NCAPD2 | MCM2 |
| USP1 | DLGAP5 | CKS2 |
| CLSPN | CDCA2 | PBK |
| POLA1 | CDCA8 | TPX2 |
| CHAF1B | ECT2 | RPL39L |
| BRIP1 | KIF23 | SNRNP25 |
| E2F8 | HMMR | TUBG1 |
| | AURKA | RNASEH2A |
| | PSRC1 | TOP2A |
| | ANLN | DTYMK |
| | LBR | RFC3 |
| | CKAP5 | CENPF |
| | CENPE | NUF2 |
| | CTCF | BUB1 |
| | NEK2 | H2AFZ |
| | G2E3 | NUDT1 |
| | GAS2L3 | SMC4 |
| | CBX5 | ANLN |
| | CENPA | RFC4 |
| | | RACGAP1 |
| | | KIFC1 |
| | | TUBB6 |
| | | ORC6 |
| | | CENPW |
| | | CCNA2 |
| | | EZH2 |
| | | NASP |
| | | DEK |
| | | TMPO |
| | | DSN1 |
| | | DHFR |
| | | KIF2C |
| | | TCF19 |
| | | HAT1 |
| | | VRK1 |
| | | SDF2L1 |
| | | PHF19 |
| | | SHCBP1 |
| | | SAE1 |
| | | CDCA5 |
| | | OIP5 |
| | | RANBP1 |
| | | LMNB1 |
| | | TROAP |
| | | RFC5 |
| | | DNMT1 |
| | | MSH2 |
| | | MND1 |
| | | TIMELESS |
| | | HMGB1 |
| | | ZWILCH |
| | | ASPM |
| | | ANP32E |
| | | POLA2 |
| | | FABP5 |
| | | TMEM194A |

As shown in Table 5, phase-specific genes are genes associated with G1/S or G2/M by multiple studies, including HeLa synchronization and multiple single cell analysis. As shown in Table 5, melanoma core cycling genes are those identified as being upregulated in cycling cells of both low-proliferation and low-proliferation melanoma tumors in this work. Each gene-set is ranked from most significant (top) to least significant gene (bottom) in Table 5.

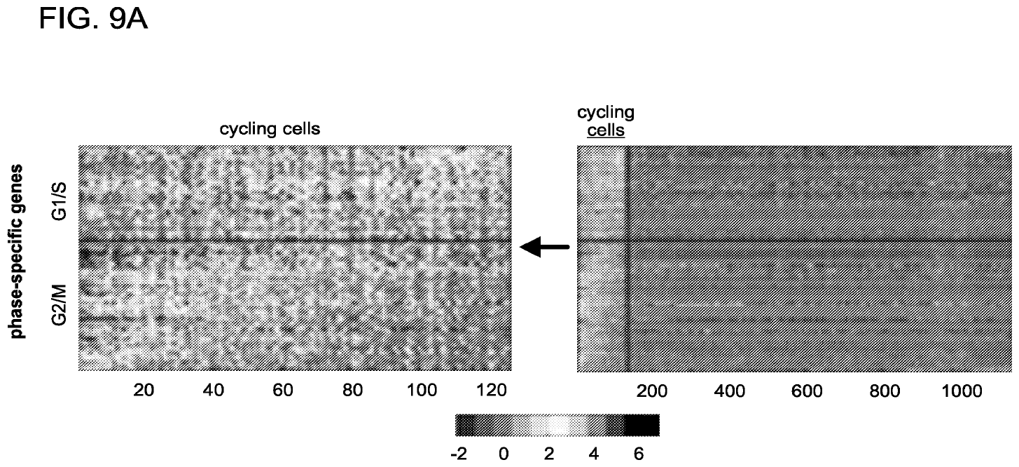

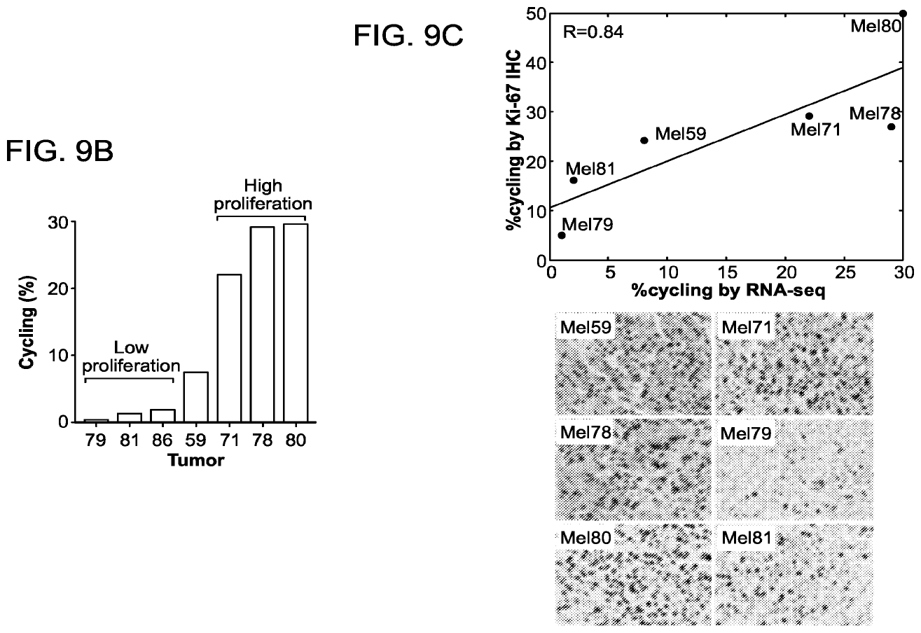

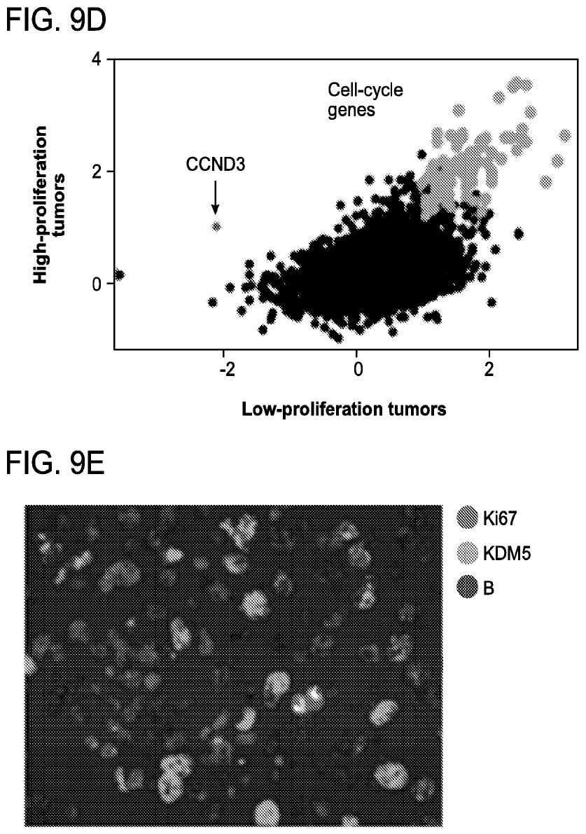

Averaging the relative expression of these gene-sets revealed cells that express primarily one of those programs, or both, while the majority of the cells do not express either of those programs (FIG. 9A-FIG. 9E). Cells were classified by the maximal expression of those two programs into non-cycling (E<1 or FDR>0.05) and cycling (E>1 and FDR<0.05) which were further divided into those with a low cell cycle signal (1<E<2), which are likely cycling but may include some false positives or arrested cells, and those with a high signal for the cell cycle (E>2) which were considered as confidently cycling cells. Of the 7 tumors for which there are >50 malignant cells, 6 have either very low (<3%) or very high (>20%) percentage of cycling malignant cells.

### Region-Specific Expression Program of Melanoma 79

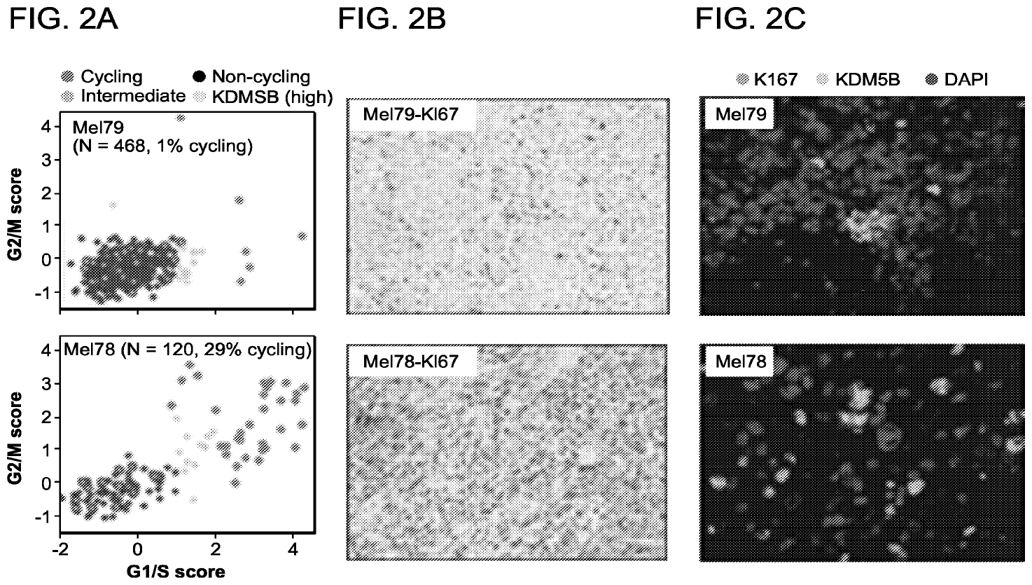

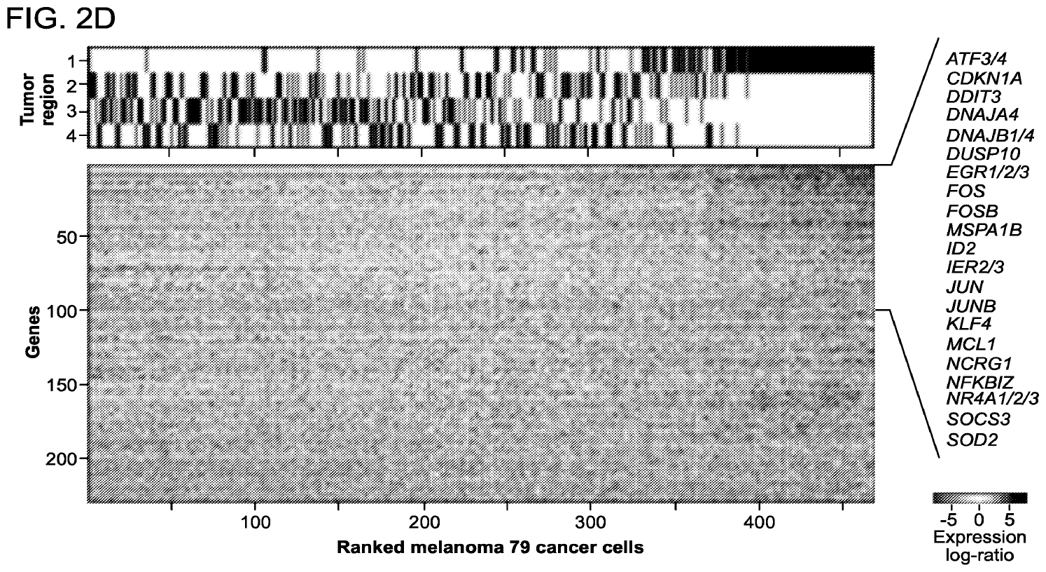

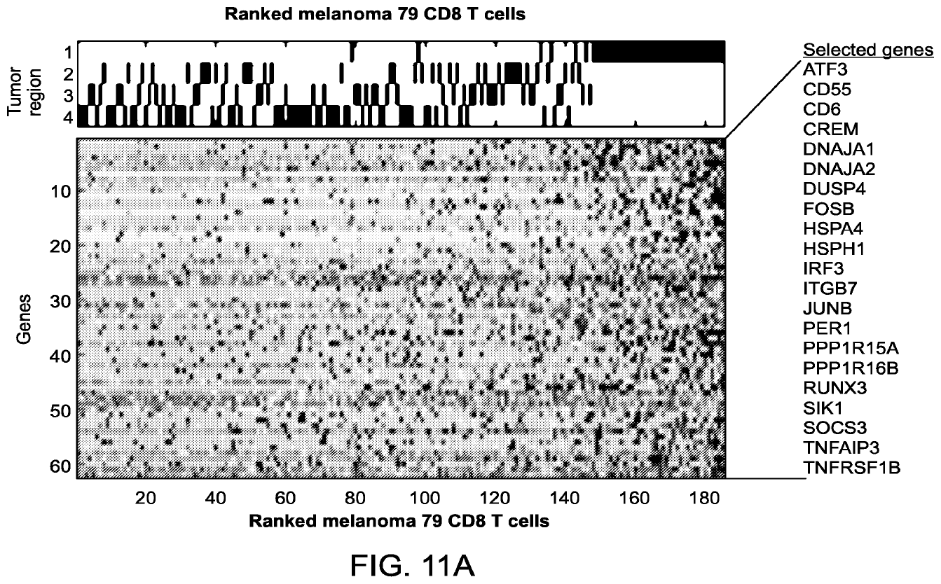

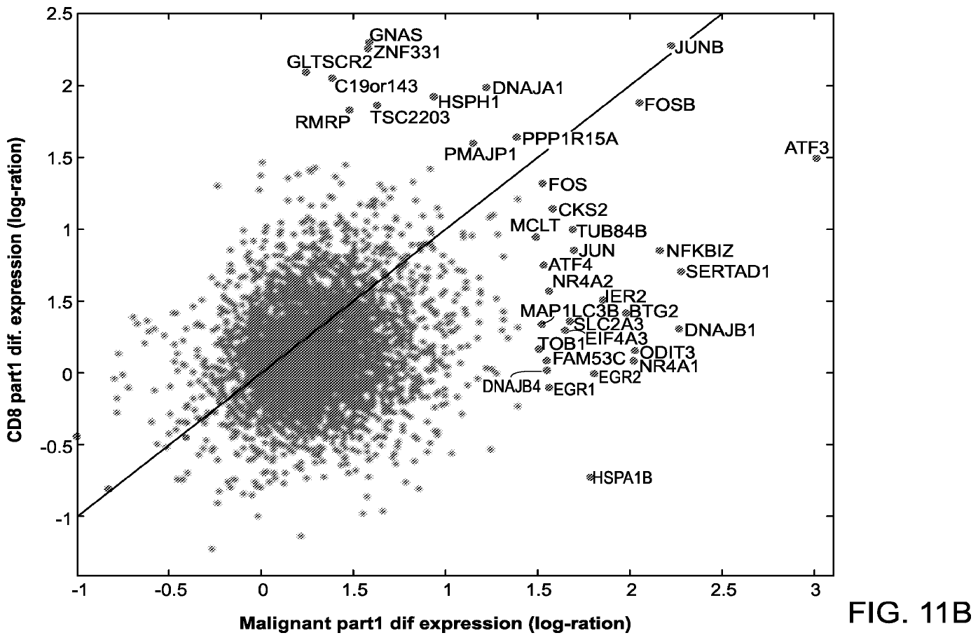

Genes with an average fold change >3 and FDR <0.05 (based both on a permutation test and a t-test with correction for multiple testing) in a comparison between either malignant (FIG. 2D) or CD8+ T (FIG. 11A and FIG. 11B) cells from Region 1 and the corresponding cells from the other parts were defined as preferentially expressed in region 1. Malignant or CD8+ T cells from Mel79 were then sorted by their average expression of these genes.

### MITF and AXL Expression Programs and Cell Scores

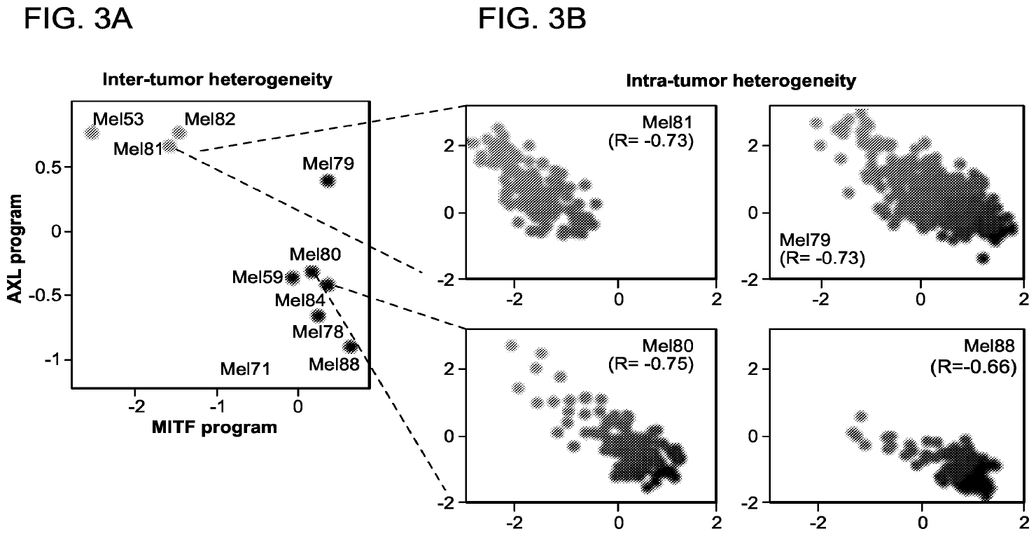

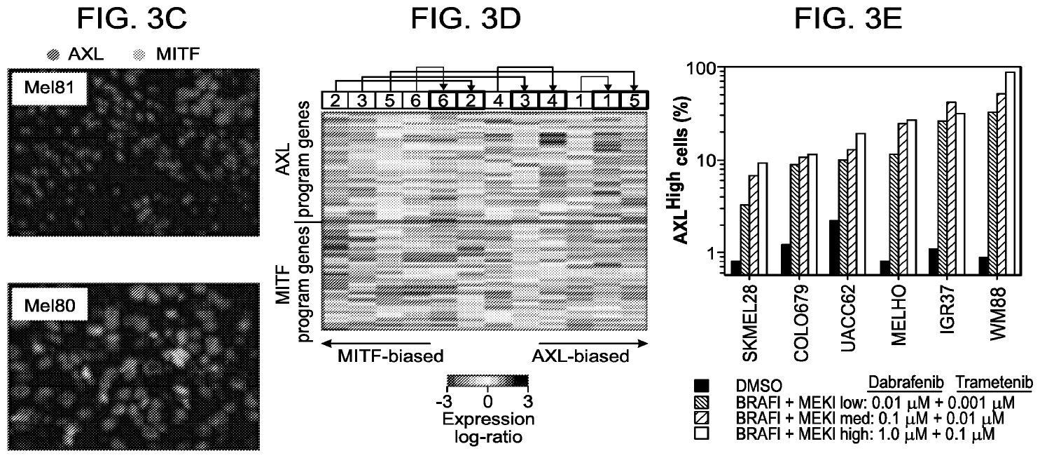

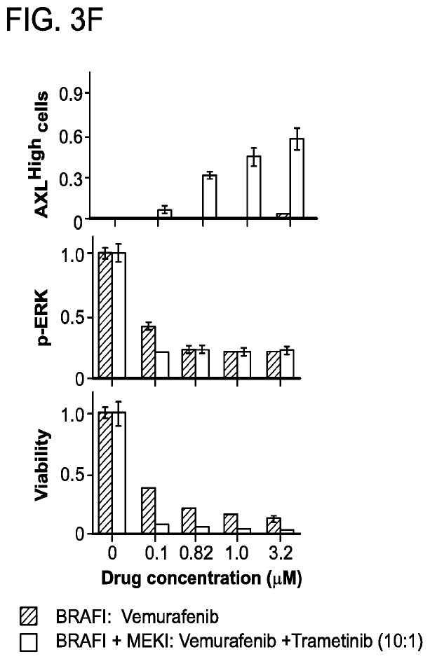

The top 100 MITF-correlated genes across the entire set of malignant cells were defined as the MITF program, and their average relative expression as the MITF-program cell score. The average expression of the top 100 genes that negatively correlate with the MITF program scores were defined as the AXL program and used to define AXL program cell score. To decrease the effect that the quality and complexity of each cell's data might have on its MITF/AXL scores control gene-sets and their average relative expression were defined as control scores, for both the MITF and AXL programs. These control cell scores were subtracted from the respective MITF/AXL cell scores. The control gene-sets were defined by first binning all analyzed genes into 25 bins of aggregate expression levels and then, for each gene in the MITF/AXL gene-set, randomly selecting 100 genes from the same expression bin as that gene. In this way, control gene-sets have a comparable distribution of expression levels to that of the MITF/AXL gene-set and the control gene set is 100-fold larger, such that its average expression is analogous to averaging over 100 randomly-selected gene-sets of the same size as the MITF/AXL gene-set. To calculate significance of the changes in AXL and MITF programs upon relapse, the expression log 2-ratio between matched pre- and post-samples for all AXL and MITF program genes was defined (FIG. 3D). Since AXL and MITF programs are inversely related, the signs of the log-ratios for MITF program genes were flipped and a t-test was used to examine if the average of the combined set of AXL program and (sign-flipped) MITF program genes is significantly higher than zero, which was the case for four out of six matched sample pairs (FIG. 3D, black arrows).

### Cell Type-Specific Signatures and Deconvolution of Bulk Expression Profiles

For each of the five main cell types identified in FIG. 1A-FIG. 1D (T cells, B cells, macrophages, endothelial cells and CAFs), cell type specific genes were defined as those: (1) with average relative expression above 3 (i.e. approximately 8-fold higher than other cells); (2) expressed by >50% of the cells in that cell type; and, (3) P<0.001 when comparing cells classified into that cell type to those in each other cell type. P values were determined for each pairwise comparison of cell types by comparing the observed fold change to that seen between 10,000 pairs of control sets. The control sets were generated such that each pair is mutually exclusive, has the same number of cells as classified to the two cell types, and each set is composed of equal number of cells from the two cell types. NK cells were not included in this analysis due to their small number and limited differences from T cells, and thus the T cell signature may also identify NK cells. Next, the melanoma TCGA RNA-seqV2 expression dataset was downloaded (Akbani et al., 2015 Cell., 161: 1681-1696) and the RSEM-based gene quantifications were log 2-transformed and the relative frequency of each cell type was estimated by the average log-transformed expression of the cell type specific genes defined above.

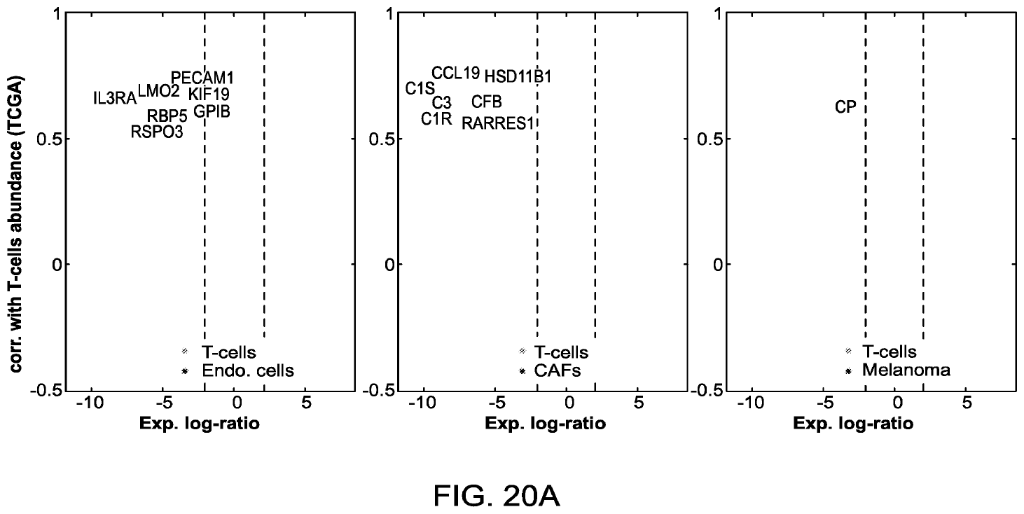

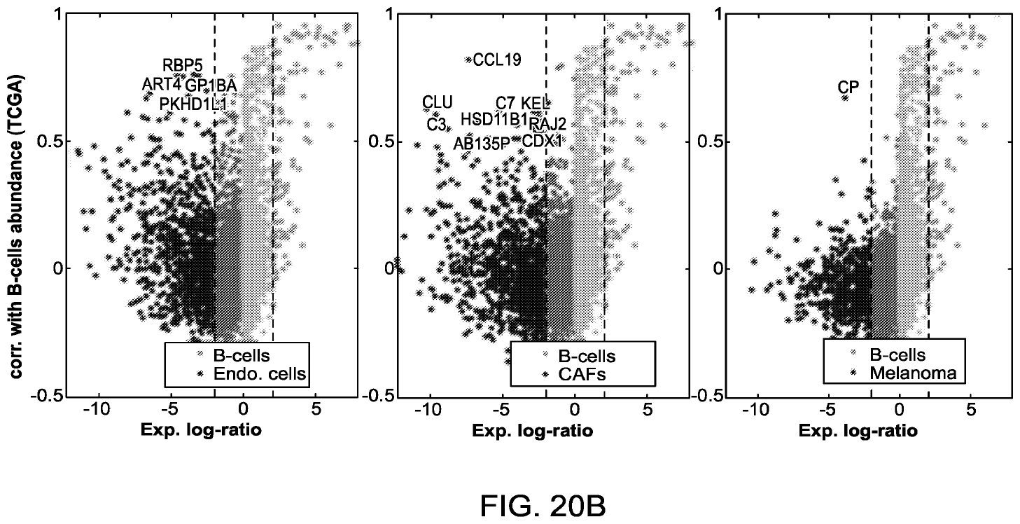

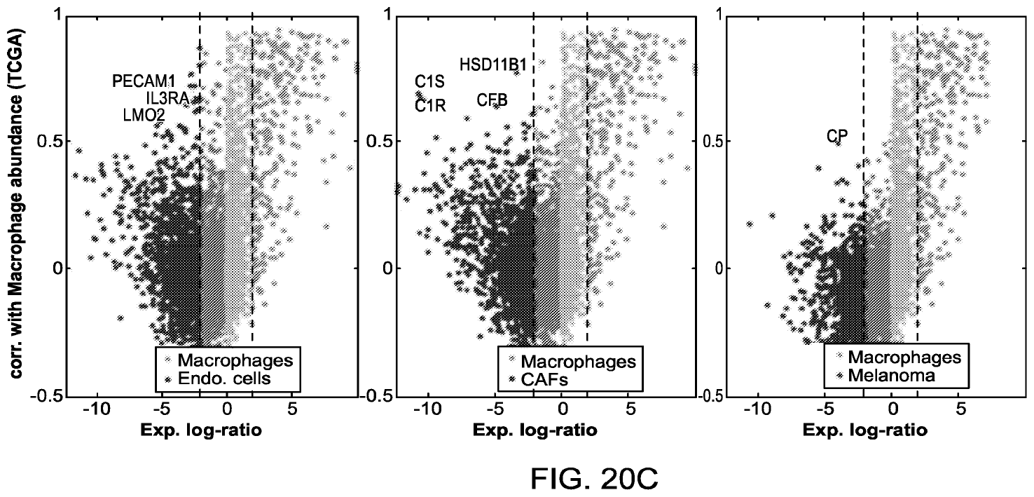

To identify genes that may mediate interactions between cell types the correlation between the expression of genes that are expressed primarily by one cell type, based on single cell profiles, and the relative frequency of another cell type, based on bulk TCGA profiles was examined. Comparison of T cells and CAFs was focused on and a set of genes was identified that although they have much higher expression in CAFs than in T cells (fold-change>4 across single cells), their expression in bulk tumors is highly correlated (R>0.5) with the estimated relative abundance of T cells (Table 13). A similar analysis was performed for all other pairs of cell-types (FIG. 20A-FIG. 20C).

TABLE 13
CAF-derived genes that correlate with the abundance of T-cells.

| CAF-expressed, | | | | |
|---|---|---|---|---|
| T/B-cell correlated | corr. | corr. | Exp (Stroma)- | Exp (Stroma)- |
| genes | With T | With B | Exp (T) | Exp (B) |
| C1S | 0.6427 | 0.5602 | 8.5056 | 9.1346 |
| UBD | 0.8315 | 0.6448 | 7.4089 | 6.6673 |
| SERPING1 | 0.654 | 0.5038 | 7.8987 | 6.7935 |
| CCL19 | 0.6804 | 0.8174 | 7.3149 | 7.7101 |
| C3 | 0.6218 | 0.6592 | 7.376 | 7.9377 |
| TGM2 | 0.5066 | 0.4779 | 7.2166 | 7.4967 |
| CXCL9 | 0.8843 | 0.6474 | 6.05 | 5.0659 |
| CXCL12 | 0.6146 | 0.6264 | 6.8387 | 7.6955 |
| TMEM176A | 0.7123 | 0.6878 | 6.5212 | 6.1329 |
| TMEM176B | 0.7597 | 0.6944 | 6.3695 | 6.355 |
| STAB1 | 0.5043 | 0.5036 | 6.9587 | 7.123 |
| CCL2 | 0.5939 | 0.5702 | 6.6362 | 6.5794 |
| PLXDC2 | 0.5126 | 0.4198 | 6.4016 | 5.8247 |
| C1R | 0.5927 | 0.5121 | 6.0416 | 8.8604 |
| CLIC2 | 0.6149 | 0.5437 | 5.9547 | 5.2628 |
| ALDH2 | 0.5594 | 0.5011 | 6.0847 | 2.554 |
| IL3RA | 0.5823 | 0.6769 | 5.7522 | 5.7951 |
| FPR2 | 0.6515 | 0.4368 | 5.518 | 5.1341 |
| SERPINA1 | 0.7051 | 0.5423 | 5.2067 | 4.9607 |
| FCGR1A | 0.7911 | 0.558 | 4.9287 | 4.8433 |
| CYBB | 0.7772 | 0.6783 | 4.9267 | −0.6677 |
| FCER1G | 0.6571 | 0.5105 | 5.2772 | 5.6419 |
| CD33 | 0.6287 | 0.5308 | 5.3447 | 4.8667 |
| LMO2 | 0.6401 | 0.6525 | 5.2456 | 2.6269 |
| SLC7A7 | 0.7918 | 0.677 | 4.7193 | 1.2406 |
| CSF1R | 0.7088 | 0.6403 | 4.7985 | 4.1882 |
| C1orf54 | 0.6741 | 0.5969 | 4.8415 | 4.1724 |
| IL34 | 0.5268 | 0.5875 | 5.2006 | 4.9851 |
| C4A | 0.5342 | 0.5331 | 5.0867 | 3.6486 |
| LILRB2 | 0.8126 | 0.6318 | 4.2076 | 3.413 |
| CSF2RB | 0.8282 | 0.8371 | 4.086 | 3.2589 |
| FPR1 | 0.6026 | 0.4769 | 4.688 | 3.4311 |
| CARD9 | 0.702 | 0.607 | 4.2483 | 3.7544 |
| TNFAIP2 | 0.721 | 0.6305 | 4.1466 | 4.1593 |
| SLCO2B1 | 0.6674 | 0.6414 | 4.2601 | 4.1278 |
| PKHD1L1 | 0.5344 | 0.6724 | 4.6243 | 3.7536 |
| FCN1 | 0.6645 | 0.5696 | 4.1683 | 3.797 |
| GP1BA | 0.586 | 0.7698 | 4.4014 | 4.1461 |
| SIGLEC6 | 0.5803 | 0.7426 | 4.4152 | 1.6201 |
| CFB | 0.6177 | 0.4997 | 4.2981 | 4.5079 |
| P2RX1 | 0.7057 | 0.7816 | 4.0268 | 1.0778 |
| NR1H3 | 0.6209 | 0.5427 | 4.2767 | 3.0717 |
| GPBAR1 | 0.7153 | 0.5332 | 3.982 | 4.0663 |
| RGS18 | 0.7173 | 0.6346 | 3.9658 | 4.0236 |
| IL7 | 0.5684 | 0.5081 | 4.3512 | 2.1569 |
| IFI30 | 0.7563 | 0.6052 | 3.7497 | 0.7839 |
| CLEC12A | 0.7339 | 0.5695 | 3.7939 | 4.7004 |
| TYROBP | 0.7613 | 0.6212 | 3.704 | 3.6344 |
| HCK | 0.8049 | 0.7162 | 3.332 | 2.0961 |
| PIK3R6 | 0.7079 | 0.6681 | 3.6123 | 2.9298 |
| ADAP2 | 0.6982 | 0.5583 | 3.6361 | 1.7039 |
| CD14 | 0.65 | 0.5399 | 3.7675 | 5.0578 |
| GHRL | 0.6626 | 0.7863 | 3.6905 | 3.8084 |
| SIGLEC9 | 0.6999 | 0.5765 | 3.5768 | 4.1243 |
| TMEM37 | 0.5852 | 0.591 | 3.8859 | 3.3609 |
| LILRA1 | 0.7067 | 0.6562 | 3.501 | 2.7022 |
| DHRS9 | 0.6137 | 0.6338 | 3.7097 | 1.8531 |
| PECAM1 | 0.6303 | 0.6685 | 3.6566 | 4.0629 |
| SPI1 | 0.782 | 0.7028 | 3.1278 | 0.44 |
| IL15RA | 0.8483 | 0.7059 | 2.904 | 5.0966 |
| SLC8A1 | 0.6955 | 0.5858 | 3.336 | 3.4454 |
| RBP5 | 0.5908 | 0.7632 | 3.6363 | 4.2231 |
| FGL2 | 0.6938 | 0.58 | 3.3051 | 3.3252 |
| MNDA | 0.7768 | 0.649 | 3.041 | 1.6354 |
| VNN1 | 0.5805 | 0.5384 | 3.6243 | 3.4418 |
| FLT3 | 0.8024 | 0.8645 | 2.9555 | 2.7583 |
| SOD2 | 0.6537 | 0.483 | 3.3772 | 3.6145 |
| CXCL11 | 0.7862 | 0.5054 | 2.9284 | 1.7897 |
| CLEC10A | 0.7288 | 0.7206 | 3.075 | 1.5159 |
| KIF19 | 0.632 | 0.5924 | 3.3161 | 3.479 |
| HSD11B1 | 0.7324 | 0.6252 | 2.9007 | 5.061 |
| CXorf21 | 0.7986 | 0.7615 | 2.6654 | 1.0901 |
| KEL | 0.5108 | 0.6335 | 3.5054 | 3.4601 |
| RARRES1 | 0.5535 | 0.5304 | 3.294 | 4.2727 |
| CFP | 0.6405 | 0.7309 | 3.0086 | 5.3814 |
| TNFSF10 | 0.7397 | 0.6063 | 2.6883 | 3.7574 |
| LILRB4 | 0.8079 | 0.6724 | 2.4161 | 2.5607 |
| P2RY12 | 0.5291 | 0.4793 | 3.2508 | 0.6342 |
| RSPO3 | 0.6312 | 0.664 | 2.8586 | 3.3143 |
| FGR | 0.7674 | 0.7263 | 2.4379 | 2.5568 |
| DRAM1 | 0.6425 | 0.4365 | 2.7659 | 1.9578 |
| ANKRD22 | 0.8067 | 0.5523 | 2.2727 | 1.9429 |
| P2RY13 | 0.83 | 0.78 | 2.1731 | 1.0301 |
| CLEC4A | 0.755 | 0.6835 | 2.3837 | 0.6484 |
| HK3 | 0.7416 | 0.5854 | 2.4237 | 2.4947 |
| FBP1 | 0.652 | 0.551 | 2.6863 | 2.8232 |
| IL18BP | 0.8309 | 0.6479 | 2.0746 | 1.5386 |
| PILRA | 0.757 | 0.6081 | 2.2904 | 2.2428 |
| TFEC | 0.776 | 0.6433 | 2.1393 | 1.1232 |
| CXCL16 | 0.5645 | 0.4462 | 2.7645 | 1.5609 |
| FCGR3A | 0.7456 | 0.4996 | 2.185 | 6.9459 |
| WARS | 0.592 | 0.3048 | 2.6364 | 2.8448 |
| LAP3 | 0.646 | 0.4136 | 2.4573 | 3.1552 |
| LGMN | 0.5569 | 0.3972 | 2.6516 | 3.0199 |
| CMKLR1 | 0.7127 | 0.6338 | 2.1556 | 1.6946 |
| RBM47 | 0.6204 | 0.5302 | 2.4299 | 1.4025 |
| SLC43A2 | 0.5629 | 0.5127 | 2.5179 | 0.8269 |
| LRRC25 | 0.7206 | 0.6321 | 2.0053 | 1.3417 |
| CP | 0.573 | 0.6772 | 2.3796 | 3.0212 |
| SLC40A1 | 0.5064 | 0.5608 | 2.4482 | 5.2851 |
| MAFB | 0.5796 | 0.4531 | 2.2015 | 2.6236 |
| CD163 | 0.622 | 0.4865 | 2.0074 | 0.9562 |
| SH2D3C | 0.5986 | 0.7095 | 2.0363 | 1.6083 |
| ODF3B | 0.5278 | 0.4128 | 2.1018 | 2.2454 |
| TLR2 | 0.5331 | 0.3832 | 2.0839 | 1.1407 |

As shown in Table 13, the first column includes the names of genes with average expression higher in CAFs than in T-cells by at least 4-fold (based on single cell data) and with a correlation of at least 0.5 with the abundance of T-cells across TCGA tumors. The second to fifth columns in Table 13 include the correlation with T and B cell abundances, and the expression difference (log-ratio) between CAF and T or B cells. Genes are sorted by the average of the fourth and fifth columns in Table 13.

### T Cell Classification

T cells were identified based on high expression of CD2 and CD3 (average of CD2, CD3D, CD3E and CD3G, E>4), and were further separated into CD4+, Tregs and CD8+ T cells based on the expression of CD4, CD25 and FOXP3, and CD8 (average of CD8A and CD8B), respectively. Naïve, cytotoxicity and exhaustion scores were estimated based on the average expression of the marker genes shown in FIG. 5B.

### T Cell Exhaustion Analysis

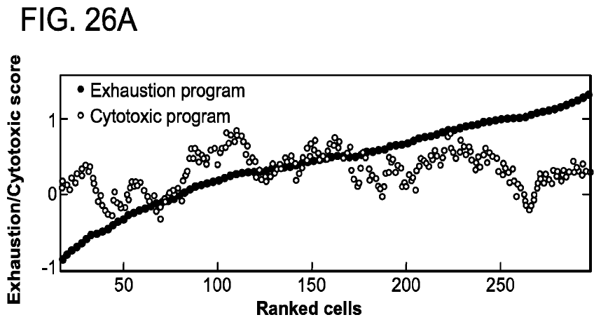

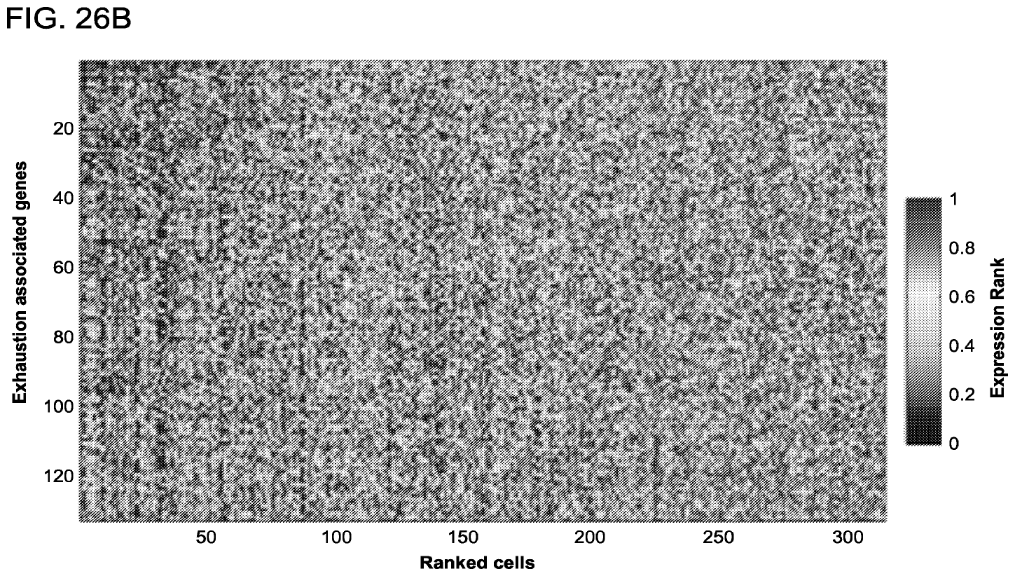

Cytotoxicity and exhaustion scores were defined as the average relative expression of cytotoxic and exhaustion gene sets, respectively, minus the average relative expression of a naïve gene-set. Cytotoxic and naïve gene-sets correspond to the genes shown in FIG. 5B, while exhaustion was estimated with each of three alternative gene-sets: (1) the program identified in Mel75 (FIG. 26A-FIG. 26B), and previously published gene-sets that represent (2) T cell exhaustion in melanoma (Baitsch et al., 2011 J. Clin. Invest., 121: 2350-2360) and (3) chronic viral infection (E. J. Wherry et al., 2007 Immunity, 27: 670-684). Importantly, even though the three gene-sets have limited overlap they give rise to similar exhaustion scores, and consequently exhaustion gene scores, as shown in FIGS. 5E-5F and Table 12, demonstrating the robustness of the analysis to the exact choice of initial exhaustion gene-sets. To estimate relative exhaustion of cells while controlling for the association between the expression of exhaustion and cytotoxicity markers, the relationship between cytotoxic and exhaustion scores was estimated using a local weighted (LOWESS) regression with a window size of 75% of the cells in each tumor (black line in FIG. 5D and FIG. 28A-FIG. 28B). Due to tumor-specific patterns, this analysis was restricted to the five tumors with more than 50 CD8 T cells. Subsets of high exhaustion cytotoxic cells (exhaustion score−regression >0.5) and low exhaustion cells (exhaustion score−regression <−0.5) were identified, and those to cells with cytotoxic scores >−3 were further restricted. These thresholds were chosen to maximize the number of genes with significantly higher expression in the high-exhaustion than in the low exhaustion subsets (P<0.001 by permutation test, as described above, and fold-change>2 in at least one tumor) (provided in Table 12). Of these, genes with P<0.05 in at least three tumors were defined as consistently associated with exhaustion and are shown in FIG. 5E. Genes with P<0.05 only in one or two tumors were defined as variably associated with exhaustion and are shown in FIG. 5F. To further evaluate the significance of differential association with exhaustion across the five tumors the observed fold-changes between high and low exhaustion cells in each individual tumor were compared to that seen in 10,000 control sets of high and low exhaustion cells that contain a mix of the different tumors with equal proportions (Table 12).

TABLE 12
Genes associated with Mel75 exhaustion signature.

| | | | |
|---|---|---|---|
| FCRL3 | IGFLR1 | ARL6IP5 | GFOD1 |
| CD27 | MGEA5 | DUSP2 | GPR174 |
| PRKCH | HSPA1B | HLA- | DDX3X |
| B2M | COTL1 | DQB1 | CAPRIN1 |
| ITM2A | VCAM1 | HNRNPK | ARPC2 |
| TIGIT | HLA-DMA | DGKH | PDIA6 |
| ID3 | PDE7B | LRMP | SEMA4A |
| GBP2 | TBC1D4 | H3F3B | CSDE1 |
| PDCD1 | SNAP47 | IDH2 | PSMB9 |
| KLRK1 | RGS4 | TRAF5 | NFATC1 |
| HSPA1A | CBLB | TBL1XR1 | |
| SRGN | TOX | ANKRD10 | |
| TNFRSF9 | CALM2 | ALDOA | |
| TMBIM6 | ATHL1 | LSP1 | |
| TNFRSF1B | SPDYE5 | PTPN7 | |
| CADM1 | DDX5 | NSUN2 | |
| ACTB | SLA | RNF149 | |
| CD8A | PTPRCAP | CD2 | |
| RGS2 | IRF9 | SRSF1 | |
| FAIM3 | MATR3 | GOLPH3 | |
| EID1 | LITAF | HLA-A | |
| HSPB1 | TPI1 | LIMS1 | |
| RNF19A | ETV1 | SDF4 | |
| IFI16 | PAM | ROCK1 | |
| LYST | ARID4B | EDEM1 | |
| PRF1 | NAB1 | APLP2 | |
| STAT1 | RAPGEF6 | ITK | |
| UBC | LDHA | TRIM22 | |
| CD74 | WARS | SPRY2 | |
| IL2RG | RASSF5 | ACTG1 | |
| FYN | OSBPL3 | HLA- | |
| PTPN6 | FAM3C | DPA1 | |
| HLA- | TAP1 | EWSR1 | |
| DRB1 | HLA- | SRSF4 | |
| HNRNPC | DRB6 | ESYT1 | |
| UBB | FABP5 | LUC7L3 | |
| CD8B | CD200 | ARNT | |
| HAVCR2 | CTLA4 | GNAS | |
| IRF8 | SNX9 | ARF6 | |
| LAG3 | ETNK1 | ARPC5L | |
| ATP5B | MALAT1 | NCOA3 | |
| STAT3 | ZDHHC6 | PAPOLA | |

### Identification of T Cell Clones

In order to detect expanded T cell clones, the transcriptome reads from each T cell were mapped to a database of TCR sequence alleles (taken from imgt.org/). Due to incomplete sequence coverage and sequencing errors, definition of the exact TCR sequence of each cell was not attempted. Instead, the usage of TCR alleles, including the V and J segments of the beta and the alpha chains was inferred. The number of reads were counted, in each cell, which were mapped by Bowtie to each of these alleles with at most one mismatch. For each segment, a cell was defined as having a certain allele if at least two reads were mapped to that allele and no other allele was supported by half as many reads or more. Cells that did not have sufficient mapped reads to a certain segment, according to this criterion, were defined as unresolved. Further analysis was restricted only to the cells with at least three resolved TCR segments out of the four that were examined (V and J of alpha and beta chains). All possible combinations of segments were examined and counted, for each combination and in each tumor, the number of cells that are consistent with it and thereby define a TCR-usage cluster. Consistency was defined as having at least three identical segments and zero inconsistent segments, in order to enable cells with one unresolved segment to be classified. Cells that were consistent with multiple, distinct combinations were assigned to the one with highest frequency. To evaluate the significance of clusters, 1,000 simulations were performed and the distribution of observed cluster sizes was compared to the combined distribution from the simulations, focusing on Mel75. In each simulation, the assignment of alleles for each segment across the Mel75 cells in which that segment was resolved was shuffled, thereby preserving the structure of the data while randomizing TCR-usage clustering. Clusters were separated to three size ranges: 1-4 cell clusters, which were not enriched in the observed TCR usage, 5-6 cell clusters, which were enriched in the observed TCR usage but with borderline significance (FDR=0.12, defined as the fraction of cells in those clusters in the control analysis divided by the fraction of cells in the observed TCR usage), and >6 cell clusters which were highly significant (FDR=0.005). Most Mel75 cells assigned to this last group were part of clusters with more than 10 cells, which were never observed in the simulations and are highly unlikely to occur by chance. Apart from Mel75, a single TCR cluster of 11 cells in Mel74 was identified (15% of cells included in TCR analysis), and no significant clusters were identified in all other tumors.

### Immunohistochemical Staining

All melanoma specimens were formalin fixed, paraffin-embedded, sectioned, and stained with hematoxylin and eosin (H&E) for histopathological evaluation at the Brigham and Women's Pathology core facility, unless otherwise specified. Immunohistochemical (IHC) studies employed 5 mm sections of formalin-fixed, paraffin-embedded tissue. All were stained on the Leica Bond III automated platform using the Leica Refine detection kit. Sections were deparaffinized and HIER was performed on the unit using EDTA for 20 minutes at 90° C. All sections were stained per routine protocols of the Brigham and Women's Pathology core facility. Additional sections were incubated for 30 min with primary antibody Ki-67 (1:250, Vector, VP-RM04) and JunB rabbit mAb (C37F9, Cell Signaling Technologies) and were then completed with the Leica Refine detection kit. The Refine detection kit encompasses the secondary antibody, the DAB chromagen (DAKO) and the Hematoxilyn counterstain. Cell counting using an ocular grid micrometer over at least five high-power fields was performed.

### Tissue Immunofluorescence Staining

Dual-labeling immunofluorescence was performed to complement immunohistochemistry as a means of two-channel identification of epitopes co-expressed in similar or overlapping sub-cellular locations. Briefly, 5-mm-thick paraffin sections were incubated with primary antibodies, AXL rabbit mAb antibody (C89E7, Abcam) plus MITF mouse mAb (clone D5, ab3201, Abcam) and JARID1B rabbit mAb (ab56759, Abcam) plus Ki67 (ab8191, Abcam) that recognize the target epitopes at 4° C. overnight and then incubated with Alexa Fluor 594-conjugated anti-mouse IgG and Alexa Fluor 488-conjugated anti-rabbit IgG (Invitrogen) at room temperature for 1 h. The sections were cover slipped with ProLong Gold anti-fade with DAPI (Invitrogen). Sections were analyzed with a BX51/BX52 microscope (Olympus America, Melville, N.Y., USA), and images were captured using the CytoVision 3.6 software (Applied Imaging, San Jose, Calif., USA). The following primary antibodies were used for staining per manufactures recommendations: mouse anti-MITF (DAKO), rabbit ant-AXL (Cell Signaling), goat anti-TIM3 (R&D Systems), rabbit ant-PD1 (Sigma Aldrich), and goat anti-PD1 (R&D Systems).

### Cell Culture Experiments and AXL Flow-Cytometry

Cell lines listed in Table 10 from the Cancer Cell Encyclopedia Lines (Barretina et al., 2012 Nature, 483: 603-607) were used for flow cytometry analysis of the proportion of AXL-positive cells.

TABLE 10
Characteristics of examined cell lines

| | MITF mRNA | AXL mRNA | Vemurafenib | Response to | | AXL expressing |
|---|---|---|---|---|---|---|
| Cell line | expression | expression | (IC50 μM) | BRAF-inhibition | BRAF mutation | cells (%) |
| IGR39 | 7.65 | 10.77 | 8 | Resistant | BRAF V600E | 98 |
| | | | | | BRAF V600E/ | |
| LOXIMVI | 5.68 | 10.43 | 8 | Resistant | I208V | 97 |
| WM793 | 6.39 | 10.05 | 8 | Resistant | BRAF V600E | 99 |
| RPMI-7951 | 6.2 | 9.78 | 8 | Resistant | BRAF V600E | 98 |
| SKMEL24 | 7.36 | 9.74 | 5.15 | Resistant | BRAF V600E | 98 |
| A2058 | 8.71 | 9.63 | 8 | Resistant | BRAF V600E | 93 |
| Hs294T | 8.89 | 8.81 | 8 | Resistant | BRAF V600E | 93 |
| WM115 | 6.85 | 8.29 | 8 | Resistant | BRAF V600D | 94 |
| IPC298 | 10.55 | 5.9 | 8 | Resistant | NRAS Q61L | 24 |
| | | | | | NRAS Q61K/ | |
| | | | | | BRAF D287H/ | |
| SKMEL30 | 10.87 | 5.34 | 8 | Resistant | E275K | 1 |
| A375 | 7.64 | 9.33 | 0.26 | Sensitive | BRAF V600E | 96 |
| WM2664 | 10.43 | 8.19 | 1.58 | Sensitive | BRAF V600D | 98 |
| WM88 | 10.05 | 6.39 | 0.2 | Sensitive | BRAF V600E | 1 |
| UACC62 | 9.5 | 5.85 | 0.25 | Sensitive | BRAF V600E | 2 |
| MELHO | 11.15 | 4.87 | 0.31 | Sensitive | BRAF V600E | 1 |
| SKMEL28 | 10.92 | 4.87 | | Sensitive | BRAF V600E | 3 |
| Colo679 | 10.34 | 4.83 | 0.55 | Sensitive | BRAF V600E | 0 |
| IGR37 | 10.85 | 4.73 | 0.9 | Sensitive | BRAF V600E | 1 |

As shown in Table 10, for MITF mRNA and AXL mRNA, vemurafenib IC50s and mutational status were extracted from CCLE (J. Barretina et al., 2012 Nature, 483: 603-607). Cells were analyzed for the fraction of AXL-high cells using FACS. Cell lines highlighted in bold in Table 10 were subsequently used for treatment experiments and measurement of AXL-high fractions by flow-cytometry and multiplexed quantitative single-cell immunofluorescence analysis. Cell lines that are highlighted in bold in Table 10 were used for subsequent drug treatment experiments, flow-cytometry and single-cell immunofluorescence analysis.

Based on IC50 values for vemurafenib, seven cell lines that were predicted to be sensitive to MAP-kinase pathway inhibition were selected, including WM88, IGR37, MELHO, UACC62, COL0679, SKMEL28 and A375 and three cell lines predicted to be resistant, including IGR39, 294T and A2058. These ten cell lines were used for drug sensitivity testing and pre-treatment and post-treatment analysis of the AXL-positive fraction. For WM88, IGR37, MELHO, UACC62, COL0679, SKMEL28 and A375, cells were plated at a density to be at 30-50% confluent after 16 hours post seeding. A total of four drug arms were plated for each cell line using two T75 (Corning) and two T175 (Corning) culture flasks. Approximately 16-24 hours after seeding, cells were treated with DMSO or dabrafenib (D) and trametinib (T) at the following drug doses of D/T: 0.01 uM/0.001 uM, 0.1 uM/0.01 uM and 1 uM/0.1 uM (T175 reserved for higher drug concentrations). Cells were maintained in drug for a total of 5 days, at which point, cells were harvested for flow sorting. For IGR39, 294T and A2058, cells were plated at a density to be at 20-30% confluent 16 hours post seeding. Cells were treated with the DMSO or D/T at using the same doses as above and maintained in drug for a total of 10 days, at which point, cells were harvested for flow sorting. For AXL-flow sorting, cells were first washed with warm PBS, followed by an addition of 10 mM EDTA and incubated for 2 minutes at room temperature. Excess EDTA was then aspirated and cells incubated at 37° C. until cells detached from flask. Cells were resuspended in cold PBS 2% FBS and kept on ice. Cells were counted and 500,000 cells were transferred to 15 ml conical tubes (Falcon), spun down and resuspended in 1000 of cold PBS 2% FBS alone (negative control) or antibodies using manufacturers recommendations, including 1 μg of AXL antibody (AF154, R&D Systems) or 1 μg of normal goat IgG control (Isotype control, AB-108-C, R&D Systems). Cells were incubated on ice for 1 hour, then washed twice with cold PBS 2% FBS. Cells were pelleted and resuspended in 100 μl PBS 2% FBS with 50 of Goat IgG (H+L) APC-conjugated Antibody (F0108, R&D Systems) and incubated for 30 minutes at room temperature. Cells were then washed twice with cold PBS 2% FBS, pelleted and resuspended in 500 μl of PBS 2% FBS and transferred to 5 mL flow-cytometry tubes (Falcon). 1 μl of SYTOX Blue Dead Stain (ThermoFisher) was added to each sample and samples analyzed by flow cytometry. Data was analyzed using FACSDiva Version 6.2 using viable cells only (as determined by SYTOX Blue staining) and gates for AXL-positivity were set using the Isotype control set to <1%.

### Single-Cell Immunofluorescence Staining and Analysis

For single-cell immunofluorescence (single-cell IF) studies, the following cell lines from CCLE were included: WM88, MELHO, SKMEL28, COL0679, IGR39, A2058 and 294T. Cells were cultured and detached as described above, and seeded at a density of 10,000 cells per well into Costar 96-well black clear-bottom tissue culture plates (3603, Corning). Cells were treated using Hewlett-Packard (HP) D300 Digital Dispenser with vemurafenib (Selleck) alone or in combination with trametinib (Selleck) at indicated doses for 5 and 10 days. In the case of 10-day treatment, growth medium was changed after 5 days followed by immediate drug re-treatment. Cells were then fixed in 4% paraformaldehyde for 20 minutes at room temperature and washed with PBS with 0.1% Tween 20 (Sigma-Aldrich) (PBS-T), permeabilized in methanol for 10 min at room temperature, rewashed with PBS-T, and blocked in Odyssey Blocking Buffer for 1 hour at room temperature. Cells were incubated overnight at 4° C. with primary antibodies in Odyssey Blocking Buffer. The following primary antibodies with specified animal sources and catalogue numbers were used in specified dilution ratios: p-ERKT202/Y204 rabbit mAb (clone D13.14.4E, 4370, Cell Signaling Technology), 1:800, AXL goat polyclonal antibody (AF154, R&D Systems), 1:800, MITF mouse mAb (clone D5, ab3201, Abcam), 1:400. Cells were then stained with rabbit, mouse and goat secondary antibodies from Molecular Probes (Invitrogen) labeled with Alexa Fluor 647 (A31573), Alexa Fluor 488 (A21202), and Alexa Fluor 568 (A11057). Cells were washed once in PBS-T, once in PBS and were then incubated in 250 ng/ml Hoechst 33342 and 1:800 Whole Cell Stain (blue; ThermoScientific) solution for 20 min. Cells were washed twice with PBS and imaged with a 10× objective on a PerkinElmer Operetta High Content Imaging System. 9-11 sites were imaged in each well. Image segmentation, analysis and signal intensity quantitation were performed using Acapella software (Perkin Elmer). Population-average and single-cell data were analyzed using MATLAB 2014b software. Single-cell density scatter plots were generated using signal intensities for individual cells.

CAF-Melanoma Co-Cultures from Melanoma 80

Solid tumor sample was removed from the transport media (Day 1: date of procurement) and minced mechanically in DMEM culture media (ThermoScientific), 10% FCS (Gemini Bioproducts), 1% pen/strep (Life Technologies) on 10 cm culture plates (Corning Inc.) and left overnight in standard culture condition (37° C., humidified atmosphere, 5% CO₂). The liquid media in which the procured tissue was originally placed was spun down (1500 rpm) to isolate the detached cells in solution and the pelleted cells were resuspended in fresh culture media and propagated in culture flasks (Corning Inc.) (fraction 1). The minced tumor samples were removed from the 10 cm culture dishes on Day 2 and mechanically forced through 100 uM nylon mesh filters (Fisher Scientific) using syringe plungers and washed through with fresh culture media. The cells and tissue clumps were spun down in 50 ml conical tubes (BD Falcon), resuspended in fresh culture media, and propagated in culture flasks (fraction 2).

The 10 cm culture dishes in which the samples had been minced and placed overnight were washed replaced with fresh culture media so that the attached cells could be propagated (fraction 3). Cells were propagated by changing culture media every 3-4 days and passaging cells in 1:3 to 1:6 ratio using 0.05% trypsin (ThermoScientific) when the plates became 50-80% confluent.

### Tissue Microarray Staining, Image Acquisition and Analysis

Two individual melanoma tissue microarrays (TMAs) were purchased, including ME208 (US Biomax) and CC38-01-003 (Cybrdi). These contained a total of 308 core biopsies, including a total of 180 primary melanomas, 90 metastatic lesions, 18 melanomas with adjacent healthy skin and 20 healthy skin controls. Each TMA was double-stained with conjugated complement 3-FITC antibody (F0201, DAKO) and CD8-TRITC (ab17147, Abcam) per manufacturers' recommendations. Image acquisition was performed on the RareCyte CyteFinder high-throughput imaging platform (Campton et al., 2015 BMC Cancer, 15: 360). For each TMAslide, the 3-channel (DAPI/FITC/TRITC) 10× images were captured and stored as Bio-format stacks. The image stacks were background-subtracted with rolling ball method and stitched into single image montage of each channel using ImageJ. For the quantification of CD8/C3 positive area and signal intensity, the gray-scale images were converted into binary images with the Otsu thresholding method (Skaland et al., 2008 J. Clin. Pathol. 61, 68-71; Konsti et al., 2011 BMC Clin. Pathol., 11: 3). Each tissue spot was segmented manually and DAPI, C3 and CD8-positive areas and intensities were calculated using ImageJ (NIH, MD). In order to control for sample quality, core biopsies with a DAPI staining less than 10% of total area were excluded from the correlation analysis. The raw numerical data were then processed and Pearson's correlation coefficients were calculated between C3/CD8 area fraction and intensity using MATLAB 2014b software (MathWorks, MA).

### Example 2: Profiles of Individual Cells from Patient-Derived Melanoma Tumors

Single-cell RNA-seq profiles from 4,645 malignant, immune and stromal cells isolated from 19 freshly procured melanoma tumors that span a range of clinical and therapeutic backgrounds were measured (Table 1). These included ten metastases to lymphoid tissues (nine to lymph nodes and one to the spleen), eight to distant sites (five to sub-cutaneous/intramuscular tissue and three to the gastrointestinal tract) and one primary acral melanoma. Genotypic information was available for 17 of 19 tumors, of which four had activating mutations in BRAF and five in NRAS oncogenes; eight patients were BRAF/NRAS wild-type (Table 1).

TABLE 1
Characteristics of patients and samples included in this study

| | | Mutation | Pre-operative | Site of | Post-op. | Alive/ |
|---|---|---|---|---|---|---|
| Sample ID | Age/sex | status | treatment | resection | treatment | deceased |
| Melanoma_53 | 77/F | Wild-type | None | Subcutaneous | None | Alive |
| | | | | back lesion | | |
| Melanoma_58 | 67/F | Wild-type | Ipilimumab | Subcutaneous | None | Alive |
| | | | | leg lesion | | |
| Melanoma_59 | 80/M | Wild-type | None | Femoral lymph | Nivolumab. | Deceased |
| | | | | node | | |
| Melanoma_60 | 69/M | BRAF V600K | Trametinib, | Spleen | None | Alive |
| | | | ipilimumab | | | |
| Melanoma_65 | 65/M | BRAF V600E | None | Paraspinal | Neovax | Alive |
| | | | | intramuscular | | |
| Melanoma_67 | 58/M | BRAF V600E | None | Axillary lymph | None | Alive |
| | | | | node | | |
| Melanoma_71 | 79/M | NRAS Q61L | None | Transverse | None | Alive |
| | | | | colon | | |
| Melanoma_72 | 57/F | NRAS Q61R | IL-2, nivolumab, | External iliac | None | Alive |
| | | | ipilimumab + anti- | lymph node | | |
| | | | KIR-Ab | | | |
| Melanoma_74 | 63/M | n/a | Nivolumab | Terminal Ileum | None | Alive |
| Melanoma_75 | 80/M | Wild-type | Ipilimumab + | Subcutaneous | Nivolumab | Alive |
| | | | nivolumab, WDVAX | leg lesion | | |
| | | | WDVAX, | | | |
| Melanoma_78 | 73/M | NRAS Q61L | ipilimumab + | Small bowel | None | Deceased |
| | | | nivolumab | | | |
| Melanoma_79 | 74/M | Wild-type | None | Axillary lymph | None | Alive |
| | | | | node | | |
| Melanoma_80 | 86/F | NRAS Q61L | None | Axillary lymph | None | Alive |
| | | | | node | | |
| Melanoma_81 | 43/F | BRAF V600E | None | Axillary lymph | None | Alive |
| | | | | node | | |
| Melanoma_82 | 81/M | Wild-type | None | Axillary lymph | None | Alive |
| | | | | node | | |
| Melanoma_84 | 67/M | Wild-type | None | Acral primary | None | Alive |
| Melanoma_88 | 54/F | NRAS Q61L | Tremelimumab + | Cutanoues met | None | Alive |
| | | | MEDI3617 | | | |
| Melanoma_89 | 67/M | n/a | None | Axillary lymph | None | Alive |
| | | | | node | | |
| Melanoma_94 | 54/F | Wild-type | IFN, ipilimumab + | Iliac lymph node | None | Alive |
| | | | nivolumab | | | |

To isolate viable single cells suitable for high-quality single-cell RNA-seq, a rapid translational workflow was developed and implemented (FIG. 1A) (Patel et al., 2014 Science, 344: 1396-1401). Tumor tissues were processed immediately following surgical procurement, and single-cell suspensions were generated within ˜45 minutes using an experimental protocol optimized to reduce artefactual transcriptional changes introduced by disaggregation, temperature, or time (Example 1). Once in suspension, individual viable immune (CD45+) and non-immune (CD45−) cells (including malignant and stromal cells) were recovered by FACS. Next, cDNA was prepared from the individual cells, followed by library construction and massively parallel sequencing. The average number of mapped reads per cell was 150,000 (Example 1), with a median library complexity of 4,659 genes for malignant cells and 3,438 genes for immune cells, comparable to previous studies of only malignant cells from fresh glioblastoma tumors (Patel et al., 2014 Science, 344: 1396-1401).

### Example 3: Single-Cell Transcriptome Profiles Distinguish Cell States in Malignant and Non-Malignant Cells

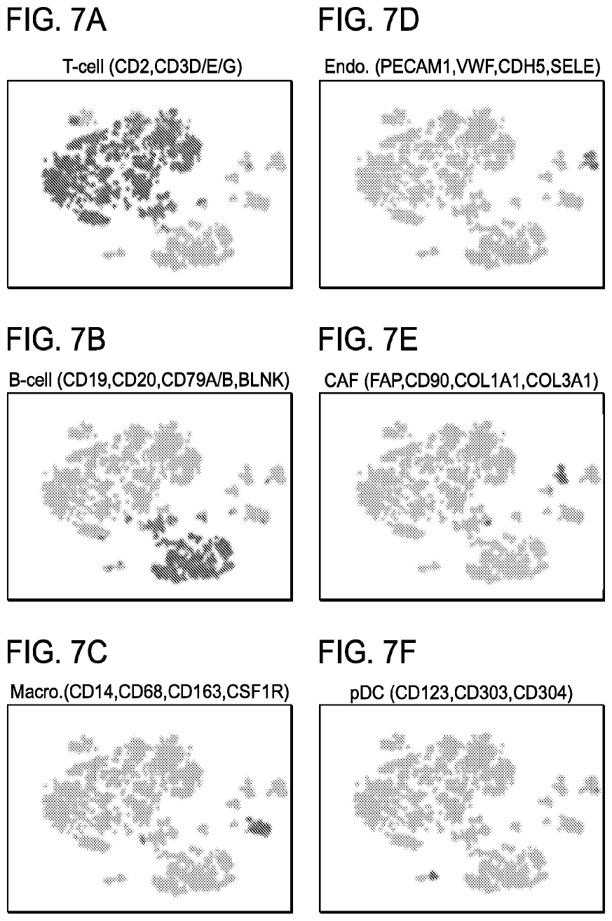

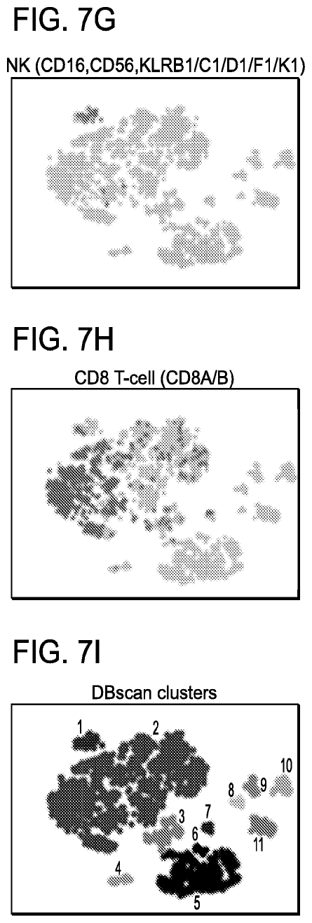

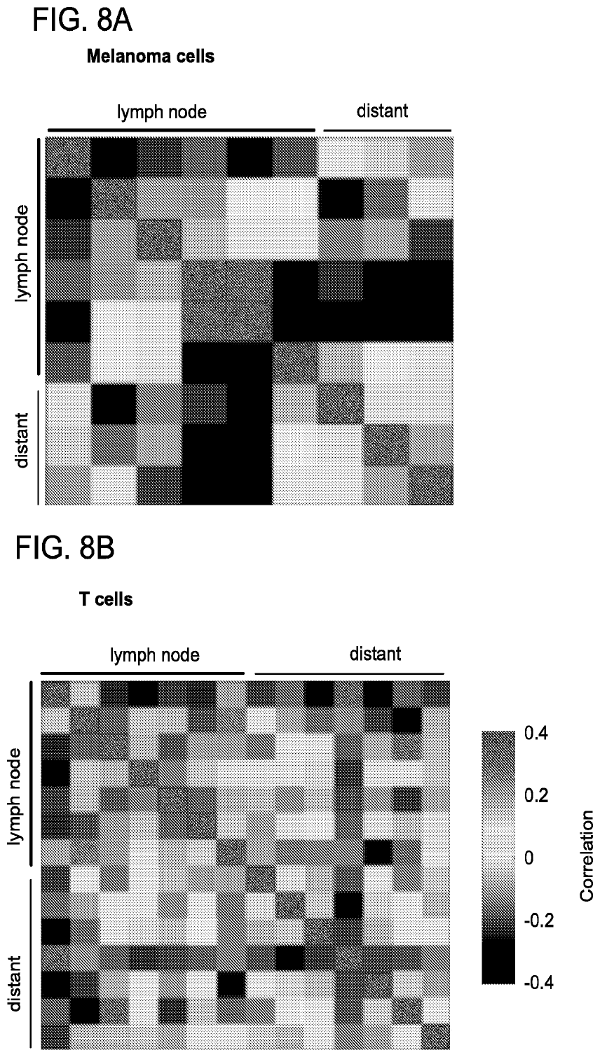

A multi-step approach was used to distinguish the different cell types within melanoma tumors based on both genetic and transcriptional states (FIG. 1B-FIG. 1D). First, large-scale copy number variations (CNVs) from expression profiles were inferred by averaging expression over 100-gene stretches on their respective chromosomes (Patel et al., 2014 Science, 344: 1396-1401) (FIG. 1B). For each tumor, this approach revealed a common pattern of aneuploidy, which was validated in two tumors by bulk whole-exome sequencing (WES, FIG. 1B and FIG. 6A). Cells in which aneuploidy was inferred were classified as malignant cells (FIG. 1B and FIG. 6A-FIG. 6B). Second, the cells were grouped based on their expression profiles (FIG. 1C-D, FIG. 7A-FIG. 7I). Here, non-linear dimensionality reduction (t-Distributed Stochastic Neighbor Embedding (t-SNE)) (L. van der Maaten and G. Hinton, 2008 Journal of Machine Learning Research, 9: 2579-2605) was used, followed by density clustering (Ester, H. Kriegel, J. Sander, and X. Xu, “A density-based algorithm for discovering clusters in large spatial databases with noise,” in Proc. 2nd Int. Conf. Knowledge Discovery and Data Mining (KDD′96), 1996, pp. 226-231). Generally, cells designated as malignant by CNV analysis formed a separate cluster for each tumor (FIG. 1C), suggesting a high degree of inter-tumor heterogeneity. In contrast, the non-malignant cells clustered by cell type (FIG. 1D and FIG. 7A-FIG. 7I), independent of their tumor of origin and metastatic site (FIG. 8A-FIG. 8B). Clusters of non-malignant cells were annotated as T cells, B cells, macrophages, endothelial cells, cancer-associated fibroblasts (CAFs) and NK cells based on preferentially or uniquely expressed marker genes (FIG. 1D, FIG. 7A-FIG. 7I, Table 2 and Table 3).

TABLE 2
Number of cells classified to each cell type in each tumor.

| | | | | Endothelial | | NK | | | |
|---|---|---|---|---|---|---|---|---|---|
| | T-cells | B-cells | Macrophages | cells | CAFs | cells | Melanoma | unclassified | Total |
| All | 2068 | 515 | 126 | 65 | 61 | 52 | 1246 | 511 | 4645 |
| tumors | | | | | | | | | |
| Mel53 | 72 | 0 | 12 | 11 | 4 | 10 | 16 | 18 | 143 |
| Mel58 | 118 | 2 | 2 | 0 | 0 | 4 | 0 | 16 | 142 |
| Mel59 | 0 | 0 | 1 | 0 | 7 | 0 | 54 | 8 | 70 |
| Mel60 | 82 | 96 | 4 | 0 | 0 | 10 | 9 | 25 | 226 |
| Mel65 | 43 | 5 | 1 | 0 | 0 | 0 | 4 | 10 | 63 |
| Mel67 | 65 | 19 | 0 | 0 | 0 | 1 | 0 | 10 | 95 |
| Mel71 | 23 | 0 | 2 | 0 | 0 | 0 | 54 | 10 | 89 |
| Mel72 | 117 | 35 | 0 | 0 | 0 | 1 | 0 | 28 | 181 |
| Mel74 | 118 | 13 | 5 | 0 | 0 | 1 | 0 | 10 | 147 |
| Mel75 | 343 | 0 | 1 | 0 | 0 | 0 | 0 | 0 | 344 |
| Mel78 | 0 | 1 | 0 | 0 | 1 | 0 | 120 | 8 | 130 |
| Mel79 | 304 | 79 | 0 | 2 | 1 | 1 | 468 | 41 | 896 |
| Mel80 | 212 | 49 | 0 | 29 | 23 | 4 | 125 | 38 | 480 |
| Mel81 | 44 | 3 | 0 | 2 | 0 | 0 | 133 | 23 | 205 |
| Mel82 | 24 | 1 | 4 | 0 | 6 | 2 | 32 | 15 | 84 |
| Mel84 | 61 | 25 | 25 | 1 | 1 | 7 | 11 | 28 | 159 |
| Mel88 | 112 | 16 | 41 | 0 | 2 | 9 | 112 | 59 | 351 |
| Mel89 | 201 | 106 | 26 | 1 | 0 | 1 | 98 | 42 | 475 |
| Mel94 | 129 | 65 | 2 | 19 | 16 | 1 | 10 | 122 | 364 |

TABLE 3
Cell type specific genes.

| | | | Endothelial | | |
|---|---|---|---|---|---|
| T-cells | B-cells | Macrophages | cells | CAFs | melanoma |
| ‘CD2’ | ‘CD19’ | ‘CD163’ | ‘PECAM1’ | ‘FAP’ | ‘MIA’ |
| ‘CD3D’ | ‘CD79A’ | ‘CD14’ | ‘VWF’ | THY1 | ‘TYR’ |
| ‘CD3E’ | ‘CD79B’ | ‘CSF1R’ | ‘CDH5’ | DCN | ‘SLC45A2’ |
| ‘CD3G’ | ‘BLK’ | ‘C1QC’ | ‘CLDN5’ | ‘COL1A1’ | ‘CDH19’ |
| ‘CD8A’ | ‘MS4A1’ | ‘VSIG4’ | ‘PLVAP’ | ‘COL1A2’ | ‘PMEL’ |
| ‘SIRPG’ | ‘BANK1’ | ‘C1QA’ | ‘ECSCR’ | ‘COL6A1’ | ‘SLC24A5’ |
| ‘TIGIT’ | ‘IGLL3P’ | ‘FCER1G’ | ‘SLCO2A1’ | ‘COL6A2’ | ‘MAGEA6’ |
| ‘GZMK’ | ‘FCRL1’ | ‘F13A1’ | ‘CCL14’ | COL6A3’ | ‘GJB1’ |
| ‘ITK’ | ‘PAX5’ | ‘TYROBP’ | ‘MMRN1’ | ‘CXCL14’ | ‘PLP1’ |
| ‘SH2D1A’ | ‘CLEC17A’ | ‘MSR1’ | ‘MYCT1’ | ‘LUM’ | ‘PRAME’ |
| ‘CD247’ | ‘CD22’ | ‘C1QB’ | ‘KDR’ | ‘COL3A1’ | ‘CAPN3’ |
| ‘PRF1’ | ‘BCL11A’ | ‘MS4A4A’ | ‘TM4SF18’ | ‘DPT’ | ‘ERBB3’ |
| ‘NKG7’ | ‘VPREB3’ | ‘FPR1’ | ‘TIE1’ | ‘ISLR’ | ‘GPM6B’ |
| ‘IL2RB’ | ‘HLA-DOB’ | ‘S100A9’ | ‘ERG’ | ‘PODN’ | ‘S100B’ |
| ‘SH2D2A’ | ‘STAP1’ | ‘IGSF6’ | ‘FABP4’ | ‘CD248’ | ‘FXYD3’ |
| ‘KLRK1’ | ‘FAM129C’ | ‘LILRB4’ | ‘SDPR’ | ‘FGF7’ | ‘PAX3’ |
| ‘ZAP70’ | ‘TLR10’ | ‘FPR3’ | ‘HYAL2’ | ‘MXRA8’ | ‘S100A1’ |
| ‘CD7’ | ‘RALGPS2’ | ‘SIGLEC1’ | ‘FLT4’ | ‘PDGFRL’ | ‘MLANA’ |
| ‘CST7’ | ‘AFF3’ | ‘LILRA1’ | ‘EGFL7’ | ‘COL14A1’ | ‘SLC26A2’ |
| ‘LAT’ | ‘POU2AF1’ | ‘LYZ’ | ‘ESAM’ | MFAP5’ | ‘GPR143’ |
| ‘PYHIN1’ | ‘CXCR5’ | ‘HK3’ | CXorf36’ | ‘MEG3’ | ‘CSPG4’ |
| ‘SLA2’ | ‘PLCG2’ | ‘SLC11A1’ | ‘TEK’ | ‘SULF1’ | ‘SOX10’ |
| ‘STAT4’ | ‘HVCN1’ | ‘CSF3R’ | ‘TSPAN18’ | ‘AOX1’ | ‘MLPH’ |
| ‘CD6’ | ‘CCR6’ | ‘CD300E’ | ‘EMCN’ | ‘SVEP1’ | ‘LOXL4’ |
| ‘CCL5’ | ‘P2RX5’ | ‘PILRA’ | ‘MMRN2’ | ‘LPAR1’ | ‘PLEKHB1’ |
| ‘CD96’ | ‘BLNK’ | ‘FCGR3A’ | ‘ELTD1’ | ‘PDGFRB’ | ‘RAB38’ |
| ‘TC2N’ | ‘KIAA0226L’ | ‘AIF1’ | ‘PDE2A’ | ‘TAGLN’ | ‘QPCT’ |
| ‘FYN’ | ‘POU2F2’ | ‘SIGLEC9’ | ‘NOS3’ | ‘IGFBP6’ | ‘BIRC7’ |
| ‘LCK’ | ‘IRF8’ | ‘FCGR1C’ | ‘ROBO4’ | ‘FBLN1’ | ‘MFI2’ |
| ‘TCF7’ | ‘FCRLA’ | ‘OLR1’ | ‘APOLD1’ | ‘CA12’ | ‘LINC00473’ |
| ‘TOX’ | ‘CD37’ | ‘TLR2’ | ‘PTPRB’ | ‘SPOCK1’ | ‘SEMA3B’ |
| ‘IL32’ | | ‘LILRB2’ | ‘RHOJ’ | ‘TPM2’ | ‘SERPINA3’ |
| ‘SPOCK2’ | | ‘C5AR1’ | ‘RAMP2’ | ‘THBS2’ | ‘PIR’ |
| ‘SKAP1’ | | ‘FCGR1A’ | ‘GPR116’ | ‘FBLN5’ | ‘MITF’ |
| ‘CD28’ | | ‘MS4A6A’ | ‘F2RL3’ | ‘TMEM119’ | ‘ST6GALNAC2’ |
| ‘CBLB’ | | ‘C3AR1’ | ‘JUP’ | ‘ADAM33’ | ‘ROPN1B’ |
| ‘APOBEC3G’ | | ‘HCK’ | ‘CCBP2’ | ‘PRRX1’ | ‘CDH1’ |
| ‘PRDM1’ | | ‘IL4I1’ | ‘GPR146’ | ‘PCOLCE’ | ‘ABCB5’ |
| | | ‘LST1’ | ‘RGS16’ | ‘IGF2’ | ‘QDPR’ |
| | | ‘LILRA5’ | ‘TSPAN7’ | ‘GFPT2’ | ‘SERPINE2’ |
| | | ‘CSTA’ | ‘RAMP3’ | ‘PDGFRA’ | ‘ATP1A1’ |
| | | ‘IFI30’ | ‘PLA2G4C’ | ‘CRISPLD2’ | ‘ST3GAL4’ |
| | | ‘CD68’ | ‘TGM2’ | ‘CPE’ | ‘CDK2’ |
| | | ‘TBXAS1’ | ‘LDB2’ | ‘F3’ | ‘ACSL3’ |
| | | ‘FCGR1B’ | ‘PRCP’ | ‘MFAP4’ | ‘NT5DC3’ |
| | | ‘LILRA6’ | ‘ID1’ | ‘C1S’ | ‘IGSF8’ |
| | | ‘CXCL16’ | ‘SMAD1’ | ‘PTGIS’ | ‘MBP’ |
| | | ‘NCF2’ | ‘AFAP1L1’ | ‘LOX’ | |
| | | ‘RAB20’ | ‘ELK3’ | ‘CYP1B1’ | |
| | | ‘MS4A7’ | ‘ANGPT2’ | ‘CLDN11’ | |
| | | ‘NLRP3’ | ‘LYVE1’ | ‘SERPINF1’ | |
| | | ‘LRRC25’ | ‘ARHGAP29’ | ‘OLFML3’ | |
| | | ‘ADAP2’ | ‘IL3RA’ | ‘COL5A2’ | |
| | | ‘SPP1’ | ‘ADCY4’ | ‘ACTA2’ | |
| | | ‘CCR1’ | ‘TFPI’ | ‘MSC’ | |
| | | ‘TNFSF13’ | ‘TNFAIP1’ | ‘VASN’ | |
| | | ‘RASSF4’ | ‘SYT15’ | ‘ABI3BP’ | |
| | | ‘SERPINA1’ | ‘DYSF’ | ‘C1R’ | |
| | | ‘MAFB’ | ‘PODXL’ | ‘ANTXR1’ | |
| | | ‘IL18’ | ‘SEMA3A’ | ‘MGST1’ | |
| | | ‘FGL2’ | ‘DOCK9’ | ‘C3’ | |
| | | ‘SIRPB1’ | ‘F8’ | ‘PALLD’ | |
| | | ‘CLEC4A’ | ‘NPDC1’ | ‘FBN1’ | |
| | | ‘MNDA’ | ‘TSPAN15’ | ‘CPXM1’ | |
| | | ‘FCGR2A’ | ‘CD34’ | ‘CYBRD1’ | |
| | | ‘CLEC7A’ | ‘THBD’ | ‘IGFBP5’ | |
| | | ‘SLAMF8’ | ‘ITGB4’ | ‘PRELP’ | |
| | | ‘SLC7A7’ | ‘RASA4’ | ‘PAPSS2’ | |
| | | ‘ITGAX’ | ‘COL4A1’ | ‘MMP2’ | |
| | | ‘BCL2A1’ | ‘ECE1’ | ‘CKAP4’ | |
| | | ‘PLAUR’ | ‘GFOD2’ | ‘CCDC80’ | |
| | | ‘SLCO2B1’ | ‘EFNA1’ | ‘ADAMTS2’ | |
| | | ‘PLBD1’ | ‘PVRL2’ | ‘TPM1’ | |
| | | ‘APOC1’ | ‘GNG11’ | ‘PCSK5’ | |
| | | ‘RNF144B’ | ‘HERC2P2’ | ‘ELN’ | |
| | | ‘SLC31A2’ | ‘MALL’ | ‘CXCL12’ | |
| | | ‘PTAFR’ | ‘HERC2P9’ | ‘OLFML2B’ | |
| | | ‘NINJ1’ | ‘PPM1F’ | ‘PLAC9’ | |
| | | ‘ITGAM’ | ‘PKP4’ | ‘RCN3’ | |
| | | ‘CPVL’ | ‘LIMS3’ | ‘LTBP2’ | |
| | | ‘PLIN2’ | ‘CD9’ | ‘NID2’ | |
| | | ‘C1orf162’ | ‘RAI14’ | ‘SCARA3’ | |
| | | ‘FTL’ | ‘ZNF521’ | ‘AMOTL2’ | |
| | | ‘LIPA’ | ‘RGL2’ | ‘TPST1’ | |
| | | ‘CD86’ | ‘HSPG2’ | ‘MIR100HG’ | |
| | | ‘GLUL’ | ‘TGFBR2’ | ‘CTGF’ | |
| | | ‘FGR’ | ‘RBP1’ | ‘RARRES2’ | |
| | | ‘GK’ | ‘FXYD6’ | ‘FHL2’ | |
| | | ‘TYMP’ | ‘MATN2’ | | |
| | | ‘GPX1’ | ‘S1PR1’ | | |
| | | ‘NPL’ | ‘PIEZO1’ | | |
| | | ‘ACSL1’ | ‘PDGFA’ | | |
| | | | ‘ADAM15’ | | |
| | | | ‘HAPLN3’ | | |
| | | | ‘APP’ | | |

Table 3 includes selected marker genes (bolded, at top) followed by all other genes defined as cell type-specific for each of the six cell types. Non-markers genes are ordered from most (top) to least (bottom) significant in Table 3, as defined by the expression difference in the respective cell type compared to all other cell types.

### Example 4: Analysis of Malignant Cells Reveals Heterogeneity in Cell Cycle and Spatial Organization

Unbiased analyses of the individual malignant cells was used to identify biologically relevant melanoma cell states. After controlling for inter-tumor differences (Example 1), the six top components from a principal component analysis were examined (PCA; Table 4). The first component correlated highly with the number of genes detected per cell, and thus likely reflects technical aspects, while the other five significant principal components highlighted biological variability.

TABLE 4
PCA. The top 50 correlated genes and the top MsigDB enrichments of those genes for the first five PCs.

| PC1 | PC2 | PC3 | PC4 | PC5 | PC1 | PC2 | PC3 | PC4 | PC5 |
|---|---|---|---|---|---|---|---|---|---|
| PPIA | PKMYT1 | PSAP | PLP1 | PLP1 | REACTOME_HOST_{—} | CELL_CYCLE_{—} | REACTOME_{—} | STRUCTURAL_{—} | PROTEIN_{—} |
| | | | | | INTERACTIONS_OF_{—} | GO_0007049 | REGULATION_OF_{—} | CONSTITUENT_{—} | HETERODIMERIZATION_{—} |
| | | | | | HIV_FACTORS | (>16) | COMPLEMENT_{—} | OF_RIBOSOME | ACTIVITY |
| | | | | | (7.8126) | | CASCADE | (5.0243) | (6.0762) |
| | | | | | | | (5.1407) | | |
| EEF1A1 | CDK1 | SERPINA3 | CAPN3 | CANX | REACTOME_{—} | REACTOME_CELL_{—} | REACTOME_INNATE_{—} | REACTOME_{—} | SPINDLE |
| | | | | | GLUCONEOGENESIS | CYCLE | IMMUNE_SYSTEM | NONSENSE_MEDIATED_{—} | (4.4747) |
| | | | | | (6.8682) | (>16) | (4.0295) | DECAY_ENHANCED_{—} | |
| | | | | | | | | BY_THE_EXON_{—} | |
| | | | | | | | | JUNCTION_COMPLEX | |
| | | | | | | | | (4.4431) | |
| CFL1 | ASF1B | CSPG4 | CDH1 | ACSL3 | KEGG_PARKINSONS_{—} | REACTOME_CELL_{—} | KEGG_ANTIGEN_{—} | SYSTEM_{—} | KEGG_LYSOSOME |
| | | | | | DISEASE | CYCLE_MITOTIC | PROCESSING_AND_{—} | DEVELOPMENT | (4.4148) |
| | | | | | (6.6129) | (>16) | PRESENTATION | (4.3937) | |
| | | | | | | | (3.8092) | | |
| MRPL12 | TK1 | LGALS3BP | ERBB3 | DDX5 | MITOCHONDRIAL_{—} | REACTOME_{—} | GLUCAN_{—} | REACTOME_SRP_{—} | MEMBRANE |
| | | | | | MEMBRANE | MITOTIC_{—} | METABOLIC_{—} | DEPENDENT_{—} | (4.4098) |
| | | | | | (6.1728) | M_M_G1_PHASES | PROCESS | COTRANSLATIONAL_{—} | |
| | | | | | | (>16) | (3.8061) | PROTEIN_TARGETING_{—} | |
| | | | | | | | | TO_MEMBRANE | |
| | | | | | | | | (4.3052) | |
| ACTG1 | CDC45 | NEAT1 | S100B | TYR | REACTOME_HIV_{—} | REACTOME_DNA_{—} | REACTOME_LIPID_{—} | PIGMENT_{—} | KEGG_MELANOGENESIS |
| | | | | | INFECTION | REPLICATION | DIGESTION_{—} | BIOSYNTHETIC_{—} | (2.8868) |
| | | | | | (6.1457) | (>16) | MOBILIZATION_{—} | PROCESS | |
| | | | | | | | AND_TRANSPORT | (4.2354) | |
| | | | | | | | (3.6338) | | |
| PSMA2 | NUSAP1 | NUCB1 | RPLP1 | QPCT | | | | | |
| PSMA6 | TOP2A | LAMB2 | PIR | MITF | | | | | |
| ATP5G3 | BUB1 | HLA-A | STK32A | PSAP | | | | | |
| ENO1 | AURKB | CTSD | TYR | CENPF | | | | | |
| LDHA | CDC6 | PLXNB2 | MLANA | ETV5 | | | | | |
| C1QBP | TPX2 | NBR1 | PMEL | RELL1 | | | | | |
| PGAM1 | CENPF | SRRM2 | SLC24A5 | ERBB3 | | | | | |
| RPLP0 | PBK | A2M | MYO10 | PTPLAD1 | | | | | |
| HSPA8 | RRM2 | FLNA | HMCN1 | BIRC5 | | | | | |
| SLC25A5 | CENPM | MTRNR2L6 | MITF | LOXL4 | | | | | |
| RAN | BIRC5 | HSPG2 | GYG2 | CALU | | | | | |
| APRT | ZWINT | AHNAK | MBP | TMEM30A | | | | | |
| TOMM5 | FANCI | DDX5 | ANKS1A | TOP2A | | | | | |
| PPP1CA | UBE2T | GAA | DCT | PTTG1IP | | | | | |
| MDH1 | TYMS | PYGB | CRYL1 | SORT1 | | | | | |
| EIF4A1 | MAD2L1 | LMNA | SEMA6A | SRSF6 | | | | | |
| NHP2 | UBE2C | GRN | SLC45A2 | PBK | | | | | |
| CDK4 | MLF1IP | MTRNR2L8 | TSPAN7 | AP1S2 | | | | | |
| PHB | KIF2C | CD276 | GPR143 | SLC12A2 | | | | | |
| RPSA | CDC20 | LTBP3 | PTPRZ1 | BUB1 | | | | | |
| ATP5A1 | RFC3 | FOSB | IGSF11 | HSPA5 | | | | | |
| NDUFAB1 | MCM4 | FOS | RPS18 | SDCBP | | | | | |
| PSMD8 | GINS2 | SLC35F5 | RPL15 | MATN2 | | | | | |
| SLC25A3 | CDKN3 | CDH19 | EXTL1 | FANCI | | | | | |
| AP2S1 | KIAA0101 | C4A | CHL1 | CNP | | | | | |
| DCTPP1 | CCNB2 | SLC38A2 | ABCB5 | SCARB2 | | | | | |
| EIF5A | CDCA7 | PC | AHCYL2 | LAMP2 | | | | | |
| ACTB | TROAP | MTRNR2L10 | LONP2 | EFNA5 | | | | | |
| AP1S1 | CCNB1 | LGMN | RPL19 | TMBIM6 | | | | | |
| COX7A2L | RACGAP1 | CD46 | SGCD | PDIA6 | | | | | |
| HNRNPF | CENPW | MTRNR2L2 | UBL3 | SLC26A2 | | | | | |
| PSMB3 | NCAPG2 | CRELD1 | VAT1 | GPNMB | | | | | |
| VDAC1 | MCM2 | TMEM87B | ASAH1 | CDC20 | | | | | |
| MRPS34 | MCM7 | CTSB | ETV5 | CD46 | | | | | |
| LDHB | MTRNR2L2 | LRP1 | CYP27A1 | ELOVL2 | | | | | |
| TUBB | ORC6 | ZNF460 | COMT | SFRP1 | | | | | |
| MDH2 | MCM5 | UBA1 | RBMS3 | ITGB1 | | | | | |
| NDUFB10 | TRIP13 | DAG1 | FCGR2C | TSPAN3 | | | | | |
| TOMM22 | EZH2 | AFAP1 | RPL7 | GPM6B | | | | | |
| SLC25A39 | MTRNR2L8 | PER1 | RPS12 | NUSAP1 | | | | | |
| MTCH2 | HMGB2 | NFKBIZ | DOCK10 | ASAH1 | | | | | |
| GOT2 | DNMT1 | P4HB | RGS20 | OSTM1 | | | | | |
| PARK7 | KIF22 | CANX | GSTP1 | HNRNPH1 | | | | | |
| CCT3 | KIF23 | ADAM10 | SCUBE2 | HPGD | | | | | |
| STOML2 | DSN1 | PROS1 | ZFP106 | CTNNB1 | | | | | |

In Table 4, significance for enriched MsigDB gene-sets is shown in parenthesis as—log 10(P), where P is the p-value from a hypergeometric test without control for multiple testing. The second component (PC2) was strongly associated with the expression of cell cycle genes (GO: “cell cycle” p<10₋₁₆; hypergeometric test). To characterize cycling cells more precisely, gene signatures previously shown to denote G1/S or G2/M phases in both synchronization (Whitfield et al., 2006 Nat. Rev. Cancer, 6: 99-106) and single cell (Macosko et al., 2015 Cell, 161: 1202-1214) experiments in cell lines were used. Cell cycle phase-specific signatures were highly expressed in a subset of malignant cells, thereby distinguishing cycling from non-cycling cells (FIG. 2A, FIG. 9A). These signatures revealed substantial variability in the fraction of cycling cells across tumors (13.5% on average, +/−13 STDV; FIG. 9B), thus allowing for designation of low-cycling tumors (1-3%, e.g. Mel79) and high-cycling ones (20-30%, e.g., Mel78) in a manner consistent with Ki67+ staining (FIG. 2B, FIG. 9C).

A core set of known cell cycle genes was robustly induced (FIG. 9D, red dots; Table 5) in both low-cycling and high-cycling tumors, with one notable exception: cyclin D3, which was only induced in cycling cells in high-cycling tumors (FIG. 9D). In contrast, KDM5B (JARID1B) showed the strongest association with non-cycling cells (FIG. 2A, green dots), mirroring recent findings in glioblastoma (Patel et al., 2014 Science, 344: 1396-1401). KDM5B encodes a H3K4 histone demethylase previously associated with a subpopulation of slow-cycling and drug-resistant melanoma stem-like cells (Roesch et al., 2010 Cell, 141: 583-594; A first-in-human phase I study of the CDK4/6 inhibitor, LY2835219, for patients with advanced cancer. J. Clin. Oncol. (available at meetinglibrary.asco.org/content/111069-132) in mouse models. Immunofluorescence (IF) staining validated the presence and mutually exclusive expression of KDM5B and Ki67 in three representative cases. KDM5B-expressing cells were grouped in small clusters, consistent with prior observations in mouse and in vitro models (Roesch et al., 2010 Cell, 141: 583-594) (FIG. 2C and FIG. 9E).

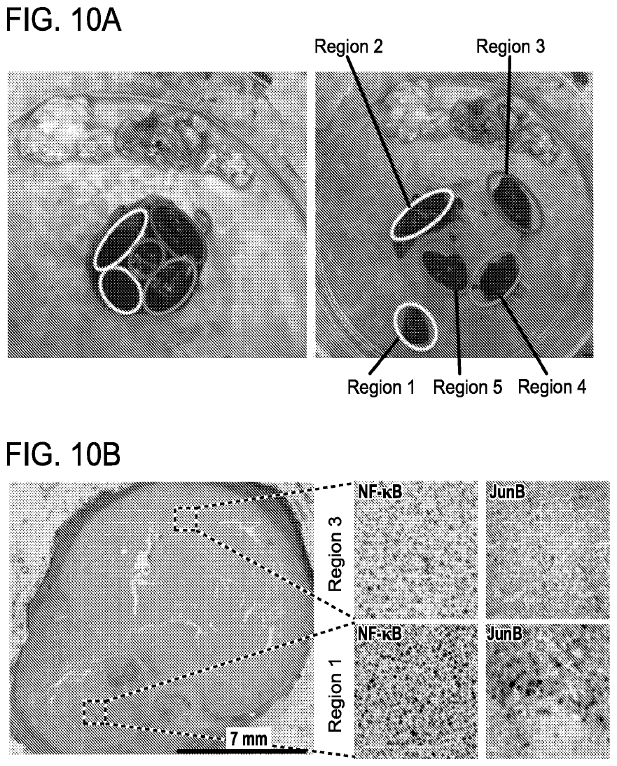

Two principal components (PC3 and PC6) primarily segregated different malignant cells from one treatment-naive tumor (Mel79). In this case, 468 malignant cells from four distinct regions that were grossly apparent following surgical resection were analyzed (FIG. 10A). 229 genes with higher expression in the malignant cells of Region 1 compared to those of other tumor regions were identified (FIG. 2D, FDR<0.05; Table 6).

TABLE 6
Differentially regulated genes in Region 1.

| | | | | log-ratio | log-ratio |
|---|---|---|---|---|---|
| Malignant | CD8T-cells | shared | Gene | (Mel) | (CD8) |
| ATF3 | SIK1 | ATF3 | GLTSCR2 | 0.252222506 | 2.086409296 |
| FAM53C | C19orf43 | DNAJA1 | GNAS | 0.591640969 | 2.29668884 |
| EGR3 | RMRP | FOSB | ZNF331 | 0.583617152 | 2.257142919 |
| NFKBIZ | FOSB | HSPH1 | C19orf43 | 0.392958905 | 2.046862888 |
| SOCS3 | ZNF331 | JUNB | CXCR4 | −0.234720422 | 1.298185954 |
| FOSB | GNAS | PER1 | PSMB8 | 0.00798707 | 1.464984759 |
| NNMT | SOCS3 | PMAIP1 | DUSP4 | −0.002156341 | 1.375588499 |
| SERTAD1 | HSPH1 | PPP1R15A | RMRP | 0.490548014 | 1.833000677 |
| NR4A2 | SLC7A5P2 | RBM25 | TERF2IP | −0.009376162 | 1.273010866 |
| PAGE5 | KIAA1967 | SOCS3 | TSC22D3 | 0.636769013 | 1.86006528 |
| BTG2 | RGCC | VPS4A | TLN1 | 0.152717856 | 1.358647995 |
| KLF4 | GLTSCR2 | | CREM | 0.201817205 | 1.387282367 |
| DNAJB1 | TXNDC11 | | EZR | 0.267418963 | 1.407425319 |
| EGR2 | BAG3 | | TMEM2 | 0.27204415 | 1.405656163 |
| CHI3L1 | CCDC6 | | C9orf78 | 0.299673685 | 1.425507336 |
| NXT2 | EIF2AK1 | | TSPAN14 | 0.146641933 | 1.204816046 |
| CDKN1A | AKNA | | IRF3 | 0.222152342 | 1.214939509 |
| SLC2A3 | RASGEF1B | | C7orf49 | 0.459724154 | 1.451861912 |
| IER3 | UHRF1BP1L | | ACTN4 | 0.030988515 | 1.018958408 |
| NDRG1 | PPP1R16B | | HSPH1 | 0.943477917 | 1.919868893 |
| PMAIP1 | PER1 | | TSPYL2 | 0.407971639 | 1.361183455 |
| NR4A1 | ABCA2 | | SSU72 | 0.11169211 | 1.047236891 |
| MKNK2 | TMEM2 | | KIAA1967 | 0.271914486 | 1.16827486 |
| PER1 | C7orf49 | | AP1M1 | 0.439153317 | 1.321805129 |
| JUNB | TLN1 | | CD82 | 0.373425907 | 1.226507799 |
| TCN1 | JUNB | | ARPC5L | 0.261759923 | 1.086112011 |
| ERRFI1 | DNAJA1 | | CALM2 | 0.392575905 | 1.216503596 |
| NPTN | HSPA4 | | LNPEP | 0.226906333 | 1.049604835 |
| NUFIP2 | PFKFB3 | | CCT7 | 0.343368561 | 1.164020045 |
| SRSF7 | HNRNPU | | RPS2 | 0.244245073 | 1.060163373 |
| FLNB | TSC22D3 | | DCUN1D1 | 0.281186721 | 1.052819979 |
| DNAJB4 | RUNX3 | | DNAJA1 | 1.243459298 | 1.986808953 |
| MAFF | RBM25 | | TBCC | 0.270680713 | 1.013704745 |
| MCL1 | GGA2 | | CACYBP | 0.332256308 | 1.030562845 |
| PLEKHO2 | STK17A | | RPS4Y1 | 0.341835417 | 1.03610437 |
| CHST11 | PMAIP1 | | HSPA4 | 0.648299255 | 1.308682493 |
| MAP1LC3B | AP1M1 | | HDHD2 | 0.428757296 | 1.087748318 |
| SOD2 | C9orf78 | | FXYD5 | 0.539723273 | 1.174656358 |
| NR4A3 | USO1 | | PPP1R2 | 0.436903991 | 1.060838747 |
| TUBB3 | HDHD2 | | RAP1A | 0.416597548 | 1.038709705 |
| CKS2 | DNAJA2 | | ELOVL5 | 0.440147531 | 1.05558358 |
| DDIT3 | TMC8 | | HNRNPU | 0.606701127 | 1.203134881 |
| BRD2 | PSIP1 | | SHISA5 | 0.675317524 | 1.271566241 |
| IER2 | DCUN1D1 | | HCP5 | 0.506778059 | 1.100752716 |
| PLK3 | DUSP4 | | DNAJA2 | 0.582617829 | 1.166210107 |
| AHR | ATF3 | | USO1 | 0.627124484 | 1.204902878 |
| TMEM87B | SPOCK2 | | KAT7 | 0.470222105 | 1.038920309 |
| TOB2 | EZR | | EIF4H | 0.718204503 | 1.281212713 |
| EIF4A3 | TNFRSF1B | | DUSP2 | 0.465159328 | 1.025965098 |
| PCOLCE | YWHAZ | | SQSTM1 | 0.621100909 | 1.175767412 |
| SRSF3 | CD6 | | MAPRE1 | 0.619909542 | 1.159791778 |
| PPP1R15B | ITGB7 | | ATP1B3 | 0.661602658 | 1.177652739 |
| IFRD1 | RALY | | SLC7A5P2 | 0.705499587 | 1.218843372 |
| HSPA1B | PPP1R15A | | SRP9 | 0.918923062 | 1.421698009 |
| PAEP | VPS4A | | HSPA5 | 0.826024014 | 1.32473009 |
| SRSF2 | IRF3 | | JTB | 0.625007024 | 1.103564385 |
| YWHAG | CD55 | | CDKN1B | 0.57956218 | 1.055799156 |
| DDX3X | TSPAN14 | | PMAIP1 | 1.15225172 | 1.590623181 |
| TUBB4B | CREM | | RALY | 0.621965264 | 1.006144968 |
| MTHFD2 | TERF2IP | | RBM25 | 0.84546767 | 1.20544395 |
| MYO18A | TNFAIP3 | | GABARAPL2 | 0.736065071 | 1.082823722 |
| SERPINA3 | TSPYL2 | | RAB1B | 0.677618564 | 1.006438143 |
| TRA2B | | | RGS2 | 0.737751668 | 1.065700384 |
| CHRAC1 | | | CD55 | 0.69614823 | 1.011412363 |
| RBBP6 | | | PPP1R15A | 1.398393554 | 1.636271424 |
| DNAJA4 | | | DAZAP2 | 0.805682011 | 1.029351499 |
| RAB40B | | | YWHAZ | 0.88036532 | 1.088449689 |
| ALG13 | | | PER1 | 0.95717598 | 1.146285155 |
| EGR1 | | | EIF4A1 | 0.973990973 | 1.094262324 |
| RBM25 | | | VPS4A | 0.924950237 | 1.000271002 |
| PPP1R15A | | | JUNB | 2.228036981 | 2.276558898 |
| LRIF1 | | | SDF4 | 1.099456791 | 0.972452083 |
| TOB1 | | | SOCS3 | 1.239274706 | 1.087520763 |
| LDHA | | | DDX3X | 1.096796724 | 0.943467729 |
| H1F0 | | | BRD2 | 1.263815773 | 1.0985856 |
| FOS | | | FOSB | 2.060611028 | 1.878494149 |
| UPP1 | | | LDHA | 1.394207126 | 1.209591342 |
| HNRNPA3 | | | PGK1 | 1.144652884 | 0.951595812 |
| SSH1 | | | FOS | 1.53277235 | 1.318830452 |
| CEACAM1 | | | SLC38A2 | 1.040614705 | 0.77273943 |
| EFNA1 | | | FLOT2 | 1.003102526 | 0.710322909 |
| AMD1 | | | SRSF2 | 1.285810804 | 0.96158808 |
| DUSP10 | | | CCNI | 1.070713553 | 0.715893832 |
| PROS1 | | | AKIRIN1 | 1.096793774 | 0.707693066 |
| ATF4 | | | CKS2 | 1.581645741 | 1.141182216 |
| FTH1P3 | | | TCP1 | 1.113847445 | 0.638168184 |
| DHX40 | | | SRSF7 | 1.317717507 | 0.805261911 |
| ID2 | | | IFRD1 | 1.067791728 | 0.545153102 |
| CSF2RA | | | SURF4 | 1.110413256 | 0.587027483 |
| CCNL1 | | | HNRNPA1 | 1.184707116 | 0.659806945 |
| SERTAD3 | | | PLEKHO2 | 1.113486778 | 0.587438196 |
| JUN | | | CHRAC1 | 1.053477445 | 0.504222939 |
| ACSL1 | | | MCL1 | 1.501994807 | 0.950243499 |
| CCNI | | | ALDOC | 1.012393692 | 0.402809545 |
| ENO2 | | | DUSP10 | 1.00727568 | 0.390859828 |
| GTF2B | | | CIB1 | 1.195183423 | 0.568896377 |
| NEK6 | | | GTF2B | 1.046787923 | 0.405238101 |
| EIF1B | | | EIF1B | 1.193725902 | 0.551475552 |
| ETF1 | | | ENO1 | 1.110590698 | 0.440872249 |
| SRPX | | | VDAC1 | 1.017453681 | 0.343048166 |
| GOLGA5 | | | IDI1 | 1.038833197 | 0.359552913 |
| NFE2L3 | | | NEU1 | 1.184051287 | 0.486397167 |
| HSPH1 | | | TUBB4B | 1.694781409 | 0.989268362 |
| IL1RAP | | | ERP29 | 1.118556397 | 0.405331526 |
| TCP1 | | | TOB2 | 1.029853524 | 0.287804928 |
| PLK2 | | | PRDX4 | 1.047159318 | 0.293500338 |
| BACE2 | | | NEK6 | 1.071948265 | 0.317890975 |
| SDF4 | | | AMD1 | 1.279559988 | 0.521787891 |
| RCN1 | | | ATF4 | 1.543509694 | 0.757455201 |
| AKIRIN1 | | | PGAM1 | 1.187387547 | 0.357996451 |
| CITED1 | | | JUN | 1.703112224 | 0.855079899 |
| CIB1 | | | PDCD6 | 1.034992857 | 0.147728358 |
| TM4SF1 | | | ID2 | 1.316019092 | 0.425227751 |
| PELI1 | | | ACSL1 | 1.088429416 | 0.179136289 |
| FLOT2 | | | HPCAL1 | 1.127133375 | 0.191786238 |
| SLC44A3 | | | MAF1 | 1.182298314 | 0.241015831 |
| PJA2 | | | SRSF3 | 1.320409005 | 0.369260711 |
| CTSL1 | | | AHSA1 | 1.000218046 | 0.045288254 |
| NUCB1 | | | HNRNPF | 1.018726232 | 0.044997905 |
| CRELD1 | | | NR4A2 | 1.557340376 | 0.572682736 |
| MAF1 | | | ENO2 | 1.309820157 | 0.303844071 |
| NASP | | | CRELD1 | 1.082740151 | 0.075309902 |
| ARL4A | | | AKR1B1 | 1.015573187 | −0.0138164 |
| JMJD6 | | | SOD2 | 1.399769308 | 0.313967521 |
| CLIC4 | | | HSPA1A | 1.339418934 | 0.2457482 |
| SLC16A3 | | | LRIF1 | 1.002232947 | −0.106726418 |
| SLC1A5 | | | P4HA1 | 1.001445952 | −0.157545039 |
| TNFRSF21 | | | TUBA1C | 1.227893762 | 0.038967014 |
| SURF4 | | | MAP1LC3B | 1.531883103 | 0.339518494 |
| TUBA1C | | | SLC16A3 | 1.12378414 | −0.084961286 |
| VDAC1 | | | NXT2 | 1.175906742 | −0.03916186 |
| TNFRSF1A | | | SLC20A1 | 1.003434105 | −0.21252674 |
| ERP29 | | | DNAJA4 | 1.258691241 | 0.025806455 |
| GEM | | | ENTPD6 | 1.07261985 | −0.161384344 |
| AAMP | | | PLK3 | 1.278283141 | −0.004143908 |
| ALX1 | | | SLC2A3 | 1.674598643 | 0.36625382 |
| IDI1 | | | NFKBIZ | 2.167024852 | 0.85413723 |
| DNAJA1 | | | IER2 | 1.85358172 | 0.511122989 |
| NEU1 | | | TOB1 | 1.509794826 | 0.160990714 |
| HNRNPF | | | EIF4A3 | 1.655647654 | 0.299055634 |
| KLF10 | | | AAMP | 1.096238379 | −0.28094529 |
| PGAM1 | | | FAM53C | 1.556239773 | 0.087173711 |
| ENTPD6 | | | ATF3 | 3.019658275 | 1.491802942 |
| C4A | | | DNAJB4 | 1.551960965 | 0.020658815 |
| HNRNPA1 | | | BTG2 | 1.981447394 | 0.419203133 |
| TCTN1 | | | SERTAD1 | 2.276633358 | 0.712186012 |
| CCDC104 | | | CCNL1 | 1.041198985 | −0.556575632 |
| HIF1A | | | TM4SF1 | 1.398409435 | −0.231349813 |
| MANF | | | EGR1 | 1.562124421 | −0.102983977 |
| SERPINE1 | | | RCN1 | 1.246442578 | −0.525372259 |
| C15orf57 | | | EGR2 | 1.80614608 | −0.00291098 |
| PTP4A1 | | | DDIT3 | 2.029133028 | 0.153613626 |
| NAMPT | | | NR4A1 | 2.028975833 | 0.090997893 |
| TSSC1 | | | DNAJB1 | 2.266772656 | 0.306027159 |
| VPS4A | | | HSPA1B | 1.785643775 | −0.720875186 |
| ALDOC | | | | | |
| NOC2L | | | | | |
| TRIB1 | | | | | |
| ODC1 | | | | | |
| P4HA1 | | | | | |
| USP11 | | | | | |
| LTA4H | | | | | |
| HIST2H4A | | | | | |
| HIST2H4B | | | | | |
| UGDH | | | | | |
| TUBB2A | | | | | |
| IFNAR2 | | | | | |
| RAB34 | | | | | |
| DGCR2 | | | | | |
| POLDIP2 | | | | | |
| SPPL2A | | | | | |
| SPP1 | | | | | |
| ADAM9 | | | | | |
| ARPC4 | | | | | |
| SLC1A4 | | | | | |
| HPCAL1 | | | | | |
| C17orf62 | | | | | |
| FAM174A | | | | | |
| PTTG1 | | | | | |
| PLEKHB2 | | | | | |
| ATP6V1D | | | | | |
| ADM | | | | | |
| LITAF | | | | | |
| COPS4 | | | | | |
| PNRC2 | | | | | |
| HIAT1 | | | | | |
| GCSH | | | | | |
| NXF1 | | | | | |
| DDRGK1 | | | | | |
| PRDX4 | | | | | |
| KDELR2 | | | | | |
| PDCD6 | | | | | |
| ACLY | | | | | |
| YPEL5 | | | | | |
| EFTUD2 | | | | | |
| BZW2 | | | | | |
| LGMN | | | | | |
| TXNRD1 | | | | | |
| TATDN1 | | | | | |
| HMGN4 | | | | | |
| AHSA1 | | | | | |
| CLK1 | | | | | |
| AKR1B1 | | | | | |
| PPAPDC1B | | | | | |
| HMG20B | | | | | |
| SLC20A1 | | | | | |
| PFKP | | | | | |
| APOA1BP | | | | | |
| RNF185 | | | | | |
| DNAJB9 | | | | | |
| SLC25A39 | | | | | |
| BUD31 | | | | | |
| PEX10 | | | | | |
| SUMO3 | | | | | |
| LRRC41 | | | | | |
| RBMX | | | | | |
| MALSU1 | | | | | |
| ZNF32 | | | | | |
| IFI35 | | | | | |
| LYPLA2 | | | | | |
| TNFRSF12A | | | | | |
| RAP1B | | | | | |
| VAMP3 | | | | | |
| PARL | | | | | |
| ORMDL3 | | | | | |
| SFT2D2 | | | | | |
| YIPF3 | | | | | |
| SLC22A18 | | | | | |
| MAGEA12 | | | | | |

Table 6 shows genes with significantly (FDR<0.05, permutation test and t-test) higher expression in part 1 than in parts 2-4 of melanoma79, sorted by their significance from most (top) to least (bottom) significant. The first three columns of Table 6 contain significant genes from analysis of malignant cells (first column) CD8 T-cells (second column) and the genes shared by both analysis (third column). The last three columns of Table 6 show differential expression values (log 2-ratio between part1 and parts 2-4) for malignant cells and for CD8 T-cells, including all genes with at least 2-fold upregulation in one of the analysis, sorted by the difference in log-ratio between CD8 and malignant cell analysis (top genes are specifically upregulated in CD8 cells, while bottom genes are more specific to malignant cells).

A similar program was found in T cells from Region 1 (FIG. 11A-FIG. 11B and Table 6), suggesting a spatial effect that influences multiple cell types. Many of these genes encode immediate-early activation transcription factors linked to inflammation, stress responses, and a melanoma oncogenic program (e.g., ATF3, FOS, FOSB, JUN, JUNB); several of these transcription factors (e.g., FOS, JUN, NR4A1/2) are also regulated by cyclic AMP/CREB signaling, which has recently been implicated as a possible MAP kinase-independent resistance module in BRAF-mutant melanomas treated with RAF/MEK inhibition (Johannessen et al., 2013 Nature. 504, 138-142). Other top genes differentially up-regulated in Region 1 included several involved in survival (MCLJ), stress responses (EGRJ/2/3, NDRG, HSPAJB), and NF-κB signaling (NFKBIZ), up-regulation of which has also been associated with resistance to RAF/MEK inhibition (Konieczkowski et al., 2014 Cancer Discov., 4: 816-827). Immunohistochemistry confirmed the increased NF-κB and JunB levels in cells of Region 1 compared to the other regions of this tumor (FIG. 10B).

### Example 5: Heterogeneity in the Abundance of a Dormant, Drug-Resistant Melanoma Subpopulation

Collectively, the above observations implied that some treatment-naive melanoma tumors may harbor malignant cell subsets less likely to respond to targeted therapy. The transcriptional programs associated with two other principal components (PC4 and PC5) identified by unbiased analysis directly support this notion. Both PC4 and PC5 were highly correlated with expression of MITF (microphthalmia-associated transcription factor), which encodes the master melanocyte transcriptional regulator and a melanoma lineage-survival oncogene (Garraway et al., 2005 Nature, 436: 117-122). Scoring genes by their correlation to MITF across single cells, a “MITF-high” program consisting of several known MITF targets, including TYR, PMEL and MLANA was identified (Table 7).

TABLE 7
List of genes included in the MITF-program from single cell analysis.

| | | | |
|---|---|---|---|
| MITF | GYG2 | CYP27A1 | PIGY |
| TYR | SDCBP | TM7SF3 | PON2 |
| PMEL | LOXL4 | PTPRZ1 | SLC19A1 |
| PLP1 | ETV5 | CNDP2 | KLF6 |
| GPR143 | C1orf85 | CTSK | MAGED1 |
| MLANA | HMCN1 | BNC2 | ERGIC3 |
| STX7 | OSTM1 | TOB1 | PIR |
| IRF4 | ALDH7A1 | CELF2 | SLC25A5 |
| ERBB3 | FOSB | ROPN1 | JUN |
| CDH1 | RAB38 | TMEM98 | ARPC1B |
| GPNMB | ELOVL2 | CTSA | SLC19A2 |
| IGSF11 | MLPH | LIMA1 | AKR7A2 |
| SLC24A5 | PLK2 | CD99 | HPGD |
| SLC45A2 | CHL1 | IGSF8 | TBC1D7 |
| RAP2B | RDH11 | FDFT1 | TFAP2A |
| ASAH1 | LINC00473 | CPNE3 | PTPLAD1 |
| MYO10 | RELL1 | SLC35B4 | SNCA |
| GRN | C21orf91 | EIF3E | GNPTAB |
| DOCK10 | SCAMP3 | TNFRSF14 | DNAJA4 |
| ACSL3 | SGK3 | VAT1 | APOE |
| SORT1 | ABCB5 | HPS5 | MTMR2 |
| QPCT | SLC7A5 | CDK2 | ATP6V1B2 |
| S100B | SIRPA | CAPN3 | C16orf62 |
| MYC | WDR91 | SUSD5 | EXOSC4 |
| LZTS1 | PIGS | ADSL | STAM |

The MITF program was defined as the 100 genes with highest correlations with the MITF gene. In Table 7, genes are sorted from most (top) to least (bottom) significant.

A second transcriptional program, negatively correlated with the MITF program and with PC4 and PC5 (P<10-24), included AXL and NGFR (p75NTR), a marker of resistance to various targeted therapies (Zhang et al., 2012 Nat. Genet., 44: 852-860; Boiko et al., 2010 Nature, 466: 133-137) and a putative melanoma cancer stem cell marker (Boiko et al., 2010 Nature, 466: 133-137), respectively (Table 8).

TABLE 8
List of genes included in the AXL-program from single cell analysis.

| | | |
|---|---|---|
| ANGPTL4 | CRIP1 | NNMT |
| FSTL3 | SLC25A37 | PPL |
| GPC1 | LCN2 | TIMP1 |
| TMSB10 | ENO2 | RHOC |
| SH3BGRL3 | PFKFB4 | GNB2 |
| PLAUR | SLC16A3 | PDXK |
| NGFR | DBNDD2 | CTNNA1 |
| SEC14L2 | LOXL2 | CD52 |
| FOSL1 | CFB | SLC2A1 |
| SERPINE1 | CADM1 | BACH1 |
| IGFBP3 | LTBP3 | ARHGEF2 |
| TNFRSF12A | CD109 | UBE2J1 |
| GBE1 | AIM2 | CD82 |
| AXL | TCN1 | ZYX |
| PHLDA2 | STRA6 | P4HA2 |
| MAP1B | C9orf89 | PEA15 |
| GEM | DDR1 | GLRX2 |
| SLC22A4 | TBC1D8 | HAPLN3 |
| TYMP | METTL7B | RAB36 |
| TREM1 | GADD45A | SOD2 |
| RIN1 | UPP1 | ESYT2 |
| S100A4 | SPATA13 | IL18BP |
| COL6A2 | GLRX | FGFRL1 |
| FAM46A | PPFIBP1 | PLEC |
| CITED1 | PMAIP1 | |
| S100A10 | COL6A1 | |
| UCN2 | JMJD6 | |
| SPHK1 | CIB1 | |
| TRIML2 | HPCAL1 | |
| S100A6 | MT2A | |
| TMEM45A | ZCCHC6 | |
| CDKN1A | IL8 | |
| UBE2C | TRIM47 | |
| ERO1L | SESN2 | |
| SLC16A6 | PVRL2 | |
| CHI3L1 | DRAP1 | |
| FN1 | MTHFD2 | |
| S100A16 | SDC4 | |

The AXL program was defined as the 100 genes with the lowest correlations (most negative) with the average expression of the MITF program genes. In Table 8, genes are sorted from most (top) to least (bottom) significant.

Thus, to a first approximation, these transcriptional programs resemble previously reported (Konieczkowski et al., 2014 Cancer Discov., 4: 816-827; Hoek et al., 2008 Cancer Res., 68: 650-656; Müller et al., 2014 Nat. Commun., 5: 5712; Li et al., 2015 Mol. Cell. Oncol., 5: 31) “MITF-high” and “MITF-low/AXL-high” (“AXL-high”) transcriptional profiles that distinguish melanoma tumors, cell lines and mice models. Notably, the “AXL-high” program has previously been linked to intrinsic resistance to RAF/MEK inhibition (Konieczkowski et al., 2014 Cancer Discov., 4: 816-827; Hoek et al., 2008 Cancer Res., 68: 650-656; Müller et al., 2014 Nat. Commun., 5: 5712).

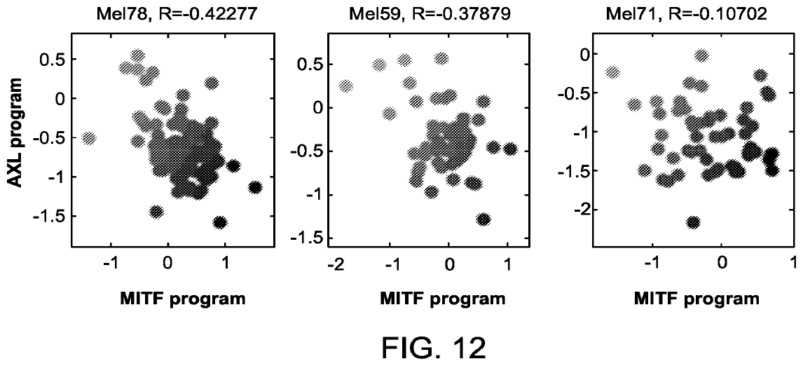

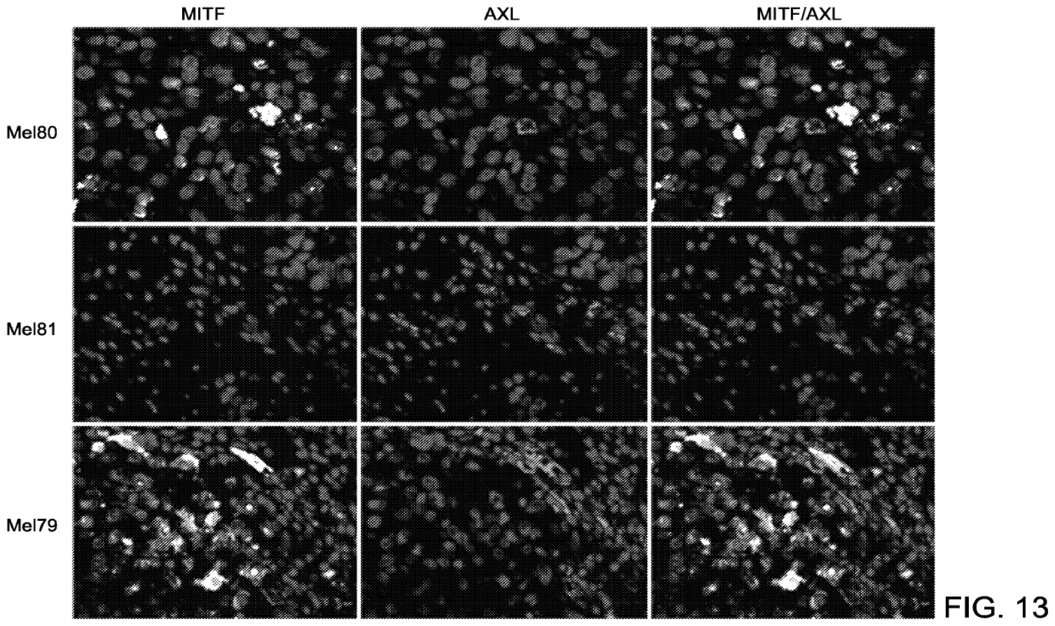

While each melanoma could be classified as “MITF-high” or “AXL-high” at the bulk tumor level (FIG. 3A), at the single cell level every tumor contained malignant cells corresponding to both transcriptional states. Using single-cell RNA-seq to examine each cell's expression of the MITF and AXL gene sets, it was observed that MITF-high tumors, including treatment-naïve melanomas, harbored a subpopulation of AXL-high melanoma cells that was undetectable through bulk analysis, and vice versa (FIG. 3B). The malignant cells thus spanned the continuum between AXL-high and MITF-high states in both (FIG. 3B and FIG. 12). The mutually exclusive expression of the MITF-high and AXL-high programs was further validated in cells from the same bulk tumors by immunofluorescence (FIG. 3C and FIG. 13).

Since malignant cells with AXL-high and MITF-high transcriptional states co-exist in melanoma, it was determined whether treatment with RAF/MEK inhibitors would increase the prevalence of AXL-high cells following the development of drug resistance. To test this, RNA-seq data from a recently published cohort (Van Allen et al., 2014 Cancer Discov, 4: 94-109) of six paired BRAFv600E melanoma biopsies taken before treatment and after resistance to single-agent RAF inhibition (vemurafenib; n=1) or combined RAF/MEK inhibition (dabrafenib and trametinib; n=5), respectively, was analyzed (Table 9).

TABLE 9
Sample information on pre-treatment and post-relapse samples

| | | Best response (in % by | PFS |
|---|---|---|---|
| Patient ID | Treatment | RECIST criteria) | (months) |
| 1 | Dabrafenib/Trametinib | −100 (CR) | 18 |
| 2 | Dabrafenib/Trametinib | −20 (SD) | 10 |
| 3 | Vemurafenib | −51 (PR) | 5 |
| 4 | Dabrafenib/Trametinib | −42 (PR) | 3 |
| 5 | Dabrafenib/Trametinib | −53 (PR) | 2 |
| 6 | Dabrafenib/Trametinib | −23 (SD) | 2 |

In Table 9, CR=complete response, PR=partial response, SD=stable disease, PFS=progression-free survival, and RECIST=Response Evaluation Criteria In Solid Tumors (Eisenhauer et al., 2009 Eur. J. Cancer Oxf. Engl., 45: 228-247).

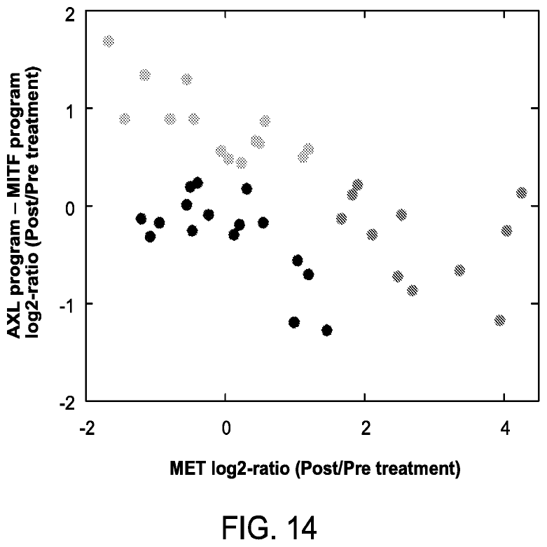

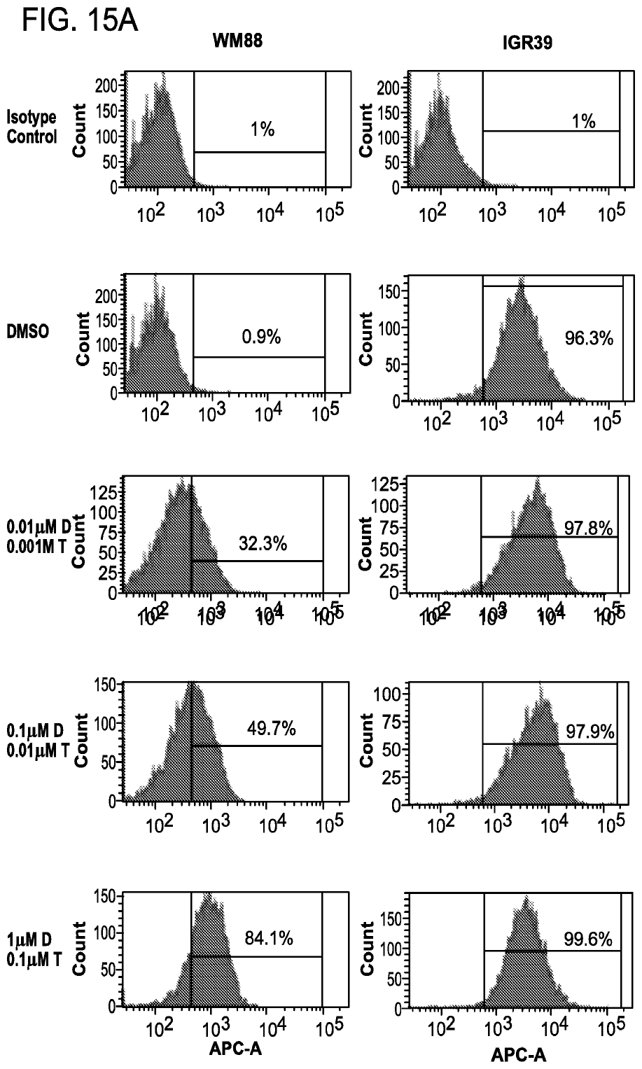

The 12 transcriptomes were ranked based on their relative expression of all genes in the AXL-high program compared to those in the MITF-high program. In each pair, a shift towards the AXL-high program was observed in the drug resistant sample, consistent with the hypothesis that AXL-high tumor cells underwent positive selection in the setting of RAF/MEK inhibition (FIG. 3D; P<0.05 for same effect in six out of six paired samples, binomial test; P<0.05 for four of six individual paired-sample comparisons shown by black arrows, Methods). RNA-seq data from an independent cohort (31) also showed that a subset of drug resistant samples exhibited increased expression of the AXL program (FIG. 14). Other genes previously implicated in resistance to RAF/MEK inhibition were also increased in a subset of the drug-resistant samples. PDGFRB(32) was upregulated in a similar subset as the AXL program, while MET (31) was upregulated in a mutually exclusive subset (FIG. 14), suggesting that AXL and MET may reflect distinct mechanisms for drug resistance.

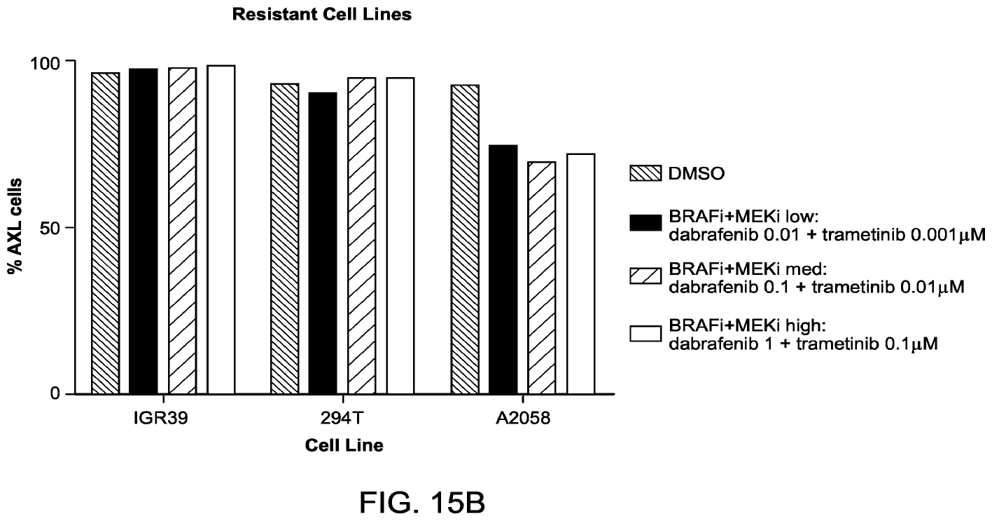

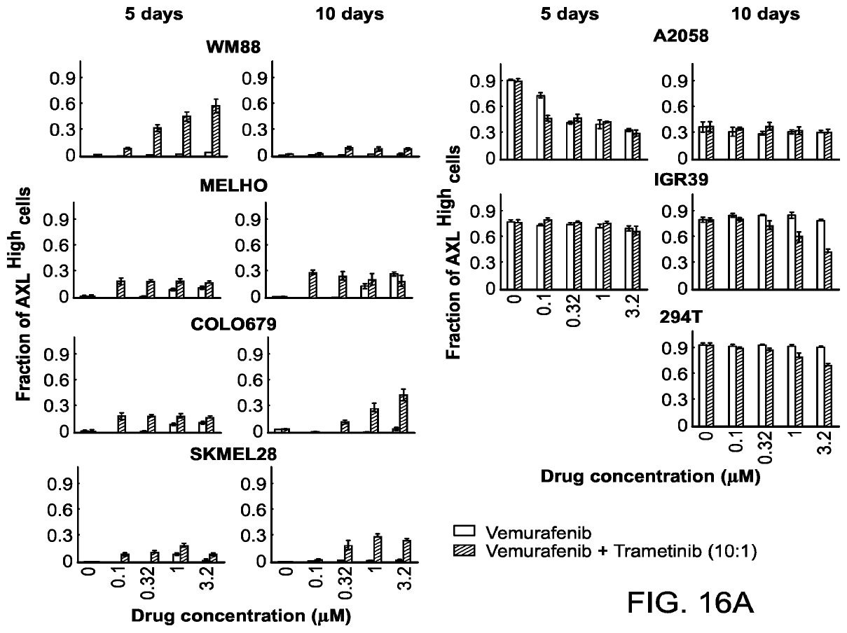

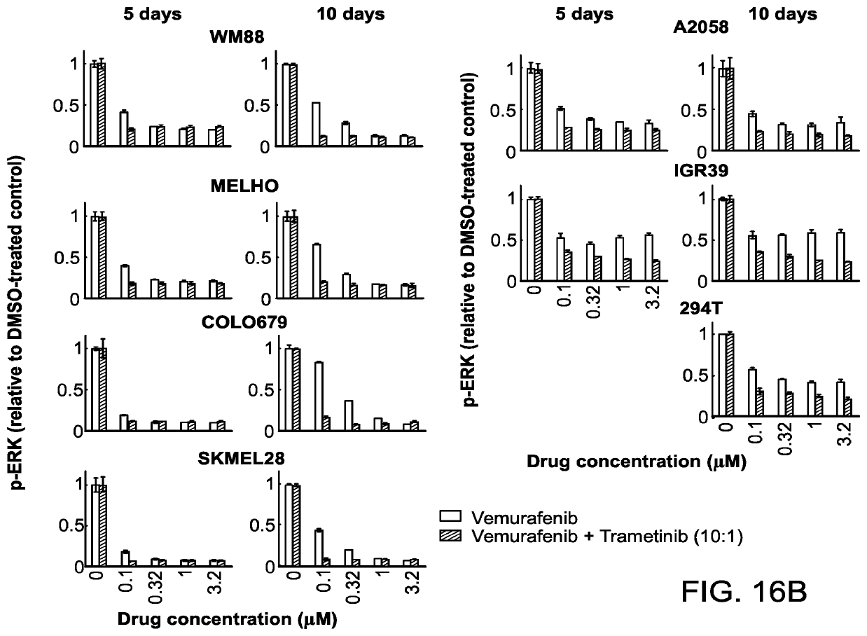

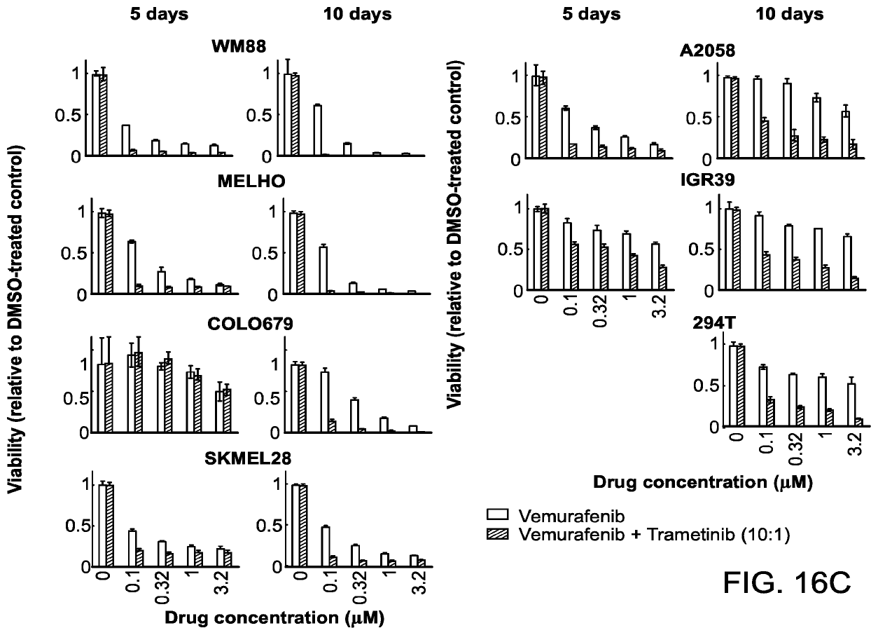

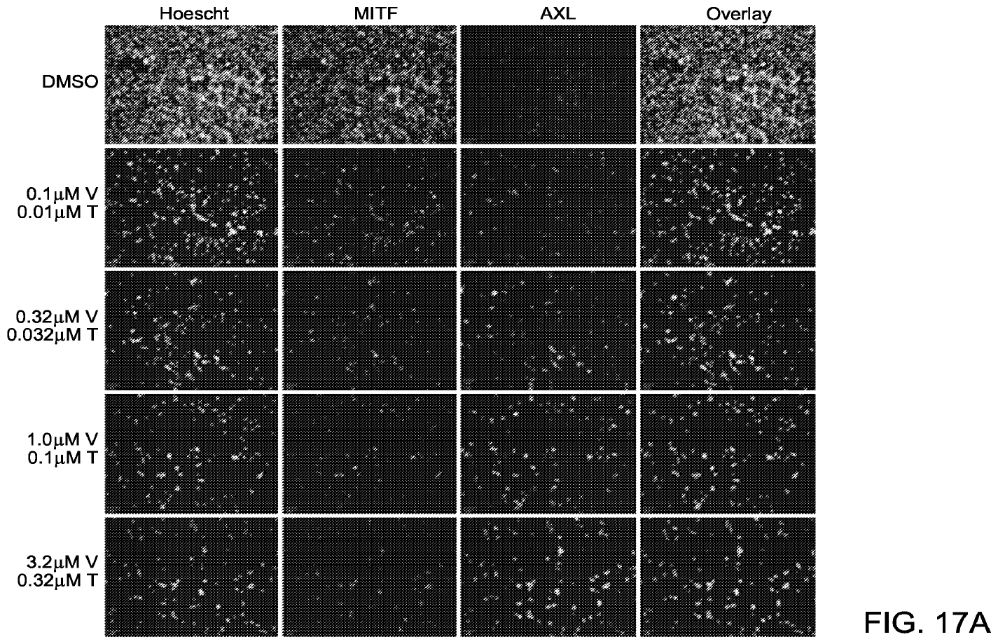

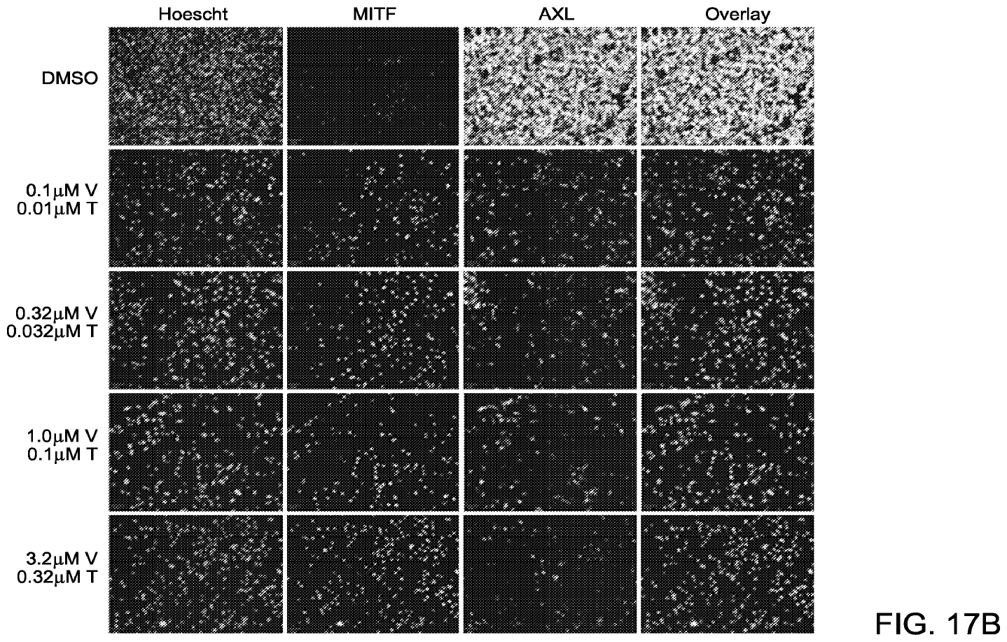

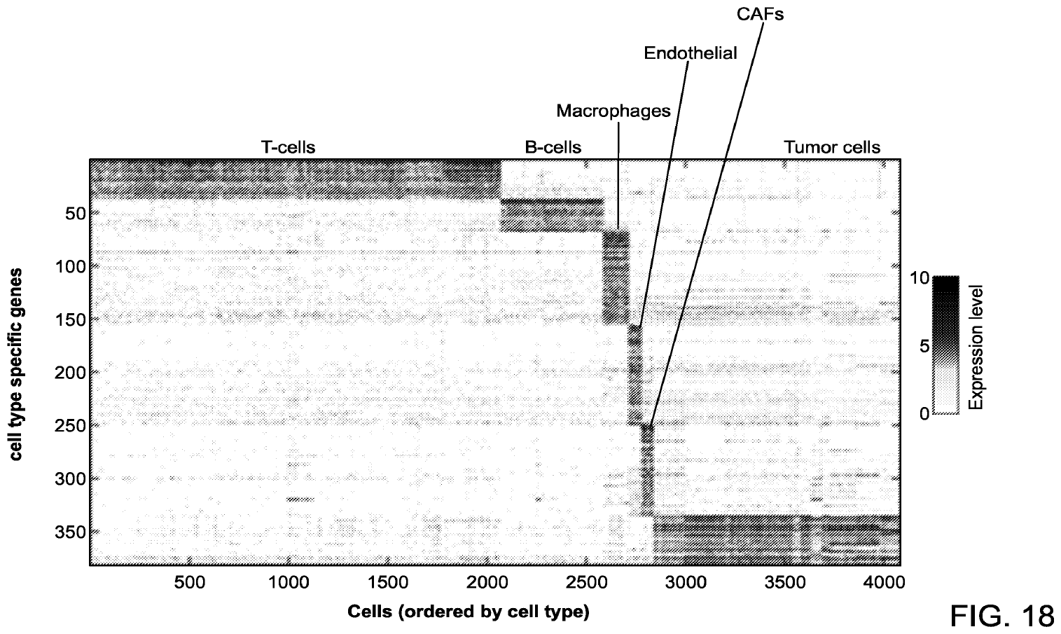

To further assess the connection between the AXL program and resistance to RAF/MEK inhibition, single-cell AXL expression was studied in 18 melanoma cell lines from the CCLE (Barretina et al., 2012 Nature, 483: 603-607) (Table 10). Flow-cytometry demonstrated a wide distribution of AXL-positive cells, from <1% to 99% per cell line, which correlated with bulk mRNA levels and were inversely associated with sensitivity to small molecule RAF inhibition (Table 10). Next, 10 cell lines (Example 1) were treated with increasing doses of a RAF/MEK inhibitor combination (dabrafenib and trametinib) (Example 1). Results showed a rapid increase in the proportion of AXL-positive cells in six cell lines with a small (<3%) pre-treatment AXL-positive population (FIG. 3E; FIG. 15A). In cell line WM88, for example, the proportion of AXL-positive cells rose from ˜1% to 84% with BRAF/MEK-inhibition (FIG. 3E; FIG. 15A-FIG. 15B, FIG. 16A-FIG. 16C, FIG. 17A-FIG. 17B). In contrast, cell lines with an intrinsically high proportion of AXL-expression, modest or no changes were observed (FIG. 15A-FIG. 15B). Similar results were obtained by multiplexed quantitative single-cell immunofluorescence (IF), which also demonstrated that the increased fraction of AXL-positive cells following RAF/MEK inhibition are associated with rapid decreases in ERK phosphorylation (reflecting MAP-kinase signaling inhibition) (FIG. 3F and FIG. 16A-FIG. 16C, FIG. 17A-FIG. 17B). In summary, studies of both melanoma tumors and cell lines demonstrate that single-cell analysis can identify drug-resistant tumor cell subpopulations that become enriched during treatment with MAP-kinase targeted treatment.

### Example 6: Non-Malignant Cells and their Interactions within the Melanoma Microenvironment

Various non-malignant cells comprise the tumor microenvironment. The composition of the microenvironment has an important impact on tumorigenesis and in the modulation of treatment responses. Tumor infiltration with T cells, for example, is predictive for the response to immune checkpoint inhibitors in various cancer types (Fridman et al., 2012 Nat. Rev. Cancer, 12: 298-306).

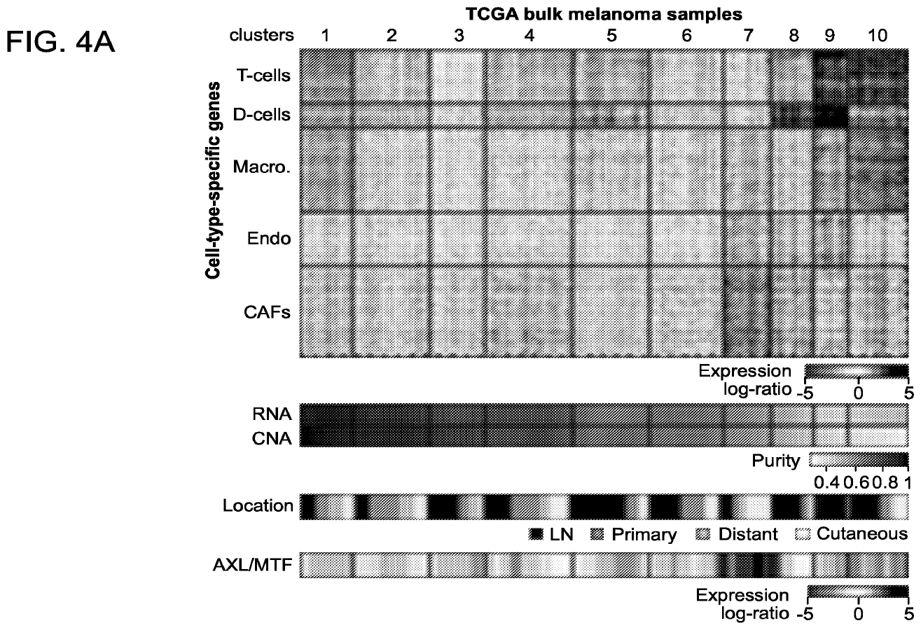

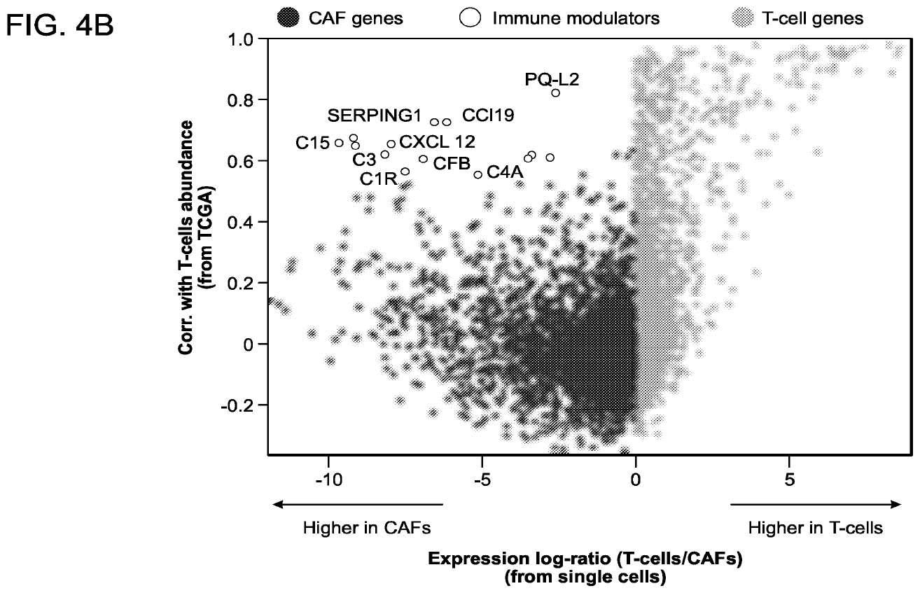

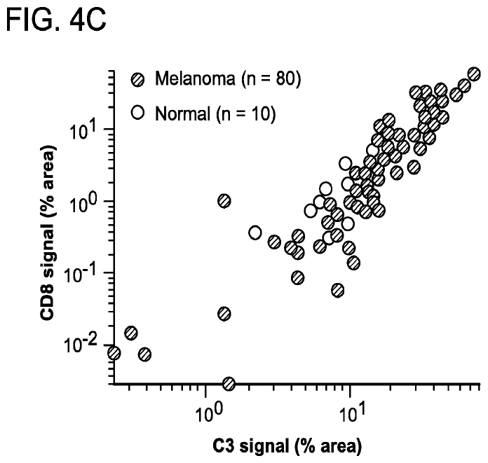

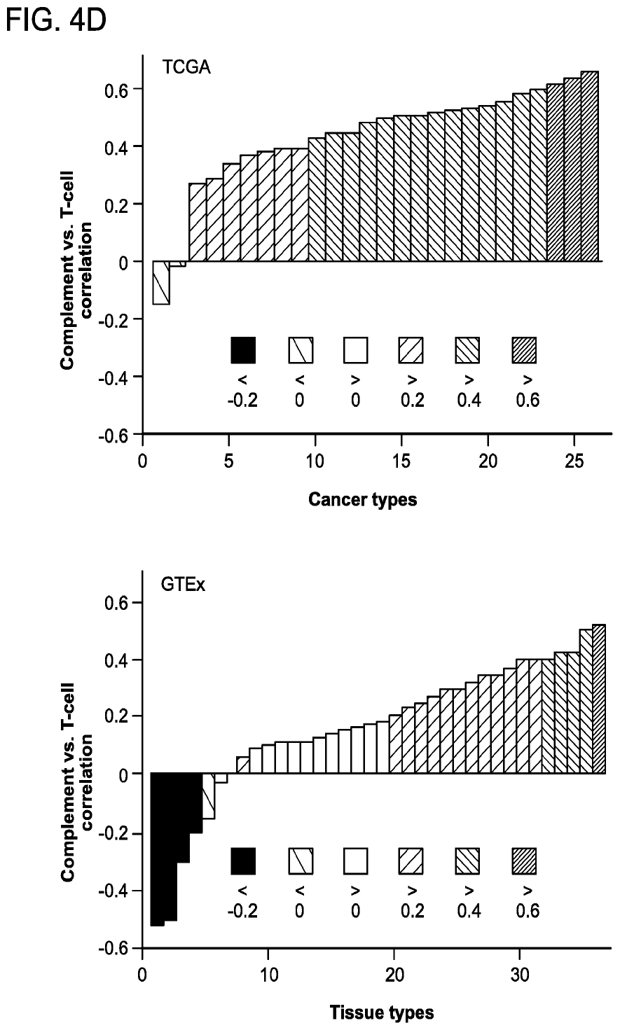

To resolve the composition of the melanoma microenvironment, the single-cell RNA-seq profiles were used to define unique expression signatures of each of five distinct non-malignant cell types: T cells, B cells, macrophages, endothelial cells, and CAFs. Because the signatures were derived from single cell profiles, it was ensured that they are based on distinct genes for each cells type, avoiding confounders (Example 1). Next, these signatures were used to infer the relative abundance of those cell types in a larger compendium of tumors published recently by the TCGA consortium (Example 1, FIG. 4A, FIG. 18). Supporting the strategy, a strong correlation (R˜0.8) was identified between the estimated tumor purity and that predicted from DNA analysis (Carter et al., 2012 Nat. Biotechnol., 30: 413-421) (FIG. 4A, first lane below the heatmap).

Using this approach, the 495 TCGA tumors were partitioned into 10 distinct microenvironment clusters based on their inferred cell type composition (FIG. 4A). For example, Cluster 9 consisted of tumors with a particularly high inferred content of B cells, whereas Cluster 4 had a relatively high inferred proportion of endothelial cells and CAFs. Clusters were mostly independent of the site of metastasis (FIG. 4A, second lane), with some notable exceptions (e.g., Clusters 8 and 9).

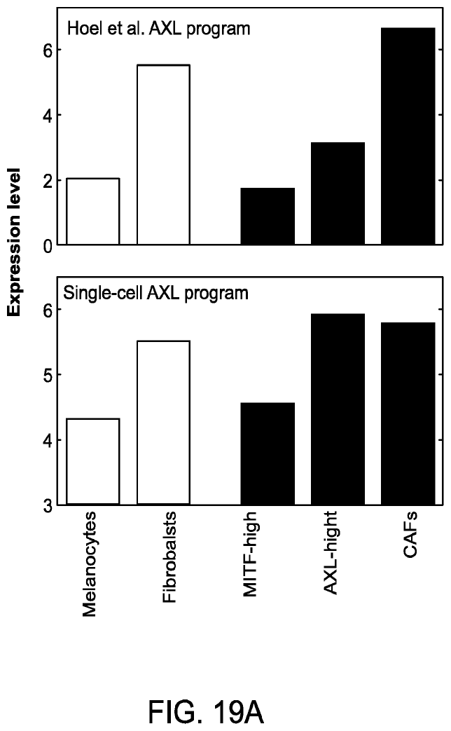

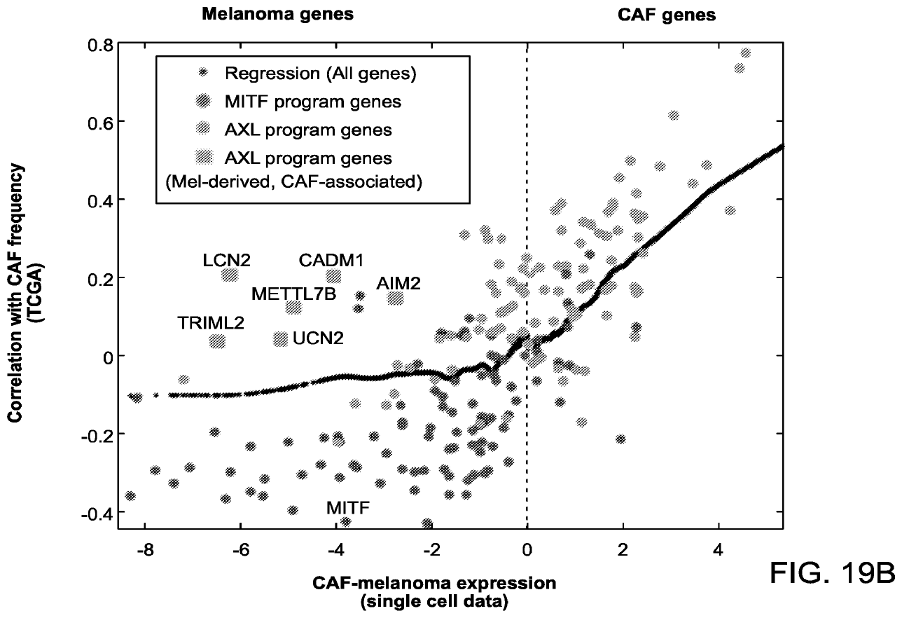

Next, it was examined how these different microenvironments may relate to the phenotype of the malignant cells. In particular, CAF abundance is predictive of the AXL-MITF distinction, such that CAF-rich tumors strongly expressed the AXL-high signature (FIG. 4A, bottom lane). An “AXL-high” program was expressed both by melanoma cells and by CAFs. However, using the single cell RNA-seq data, AXL-high genes that are preferentially expressed by melanoma cells (“melanoma-derived AXL program”) and those that are preferentially expressed by CAFs (“CAF-derived AXL program”) were distinguished. Both sets of genes were correlated with the inferred CAF abundance in TCGA tumors (FIG. 19A-FIG. 19B) (Roadmap Epigenomics Consortium et al., 2015 Nature, 518: 317-330). Furthermore, the MITF-high program, which is specific to melanoma cells, was negatively correlated with inferred CAF abundance. Taken together, these results suggest that CAF abundance is linked to preferential expression of the AXL-high over the MITF-high program within the melanoma cells. These findings raise the possibility that specific tumor-CAF interactions shapes the melanoma cell transcriptome.

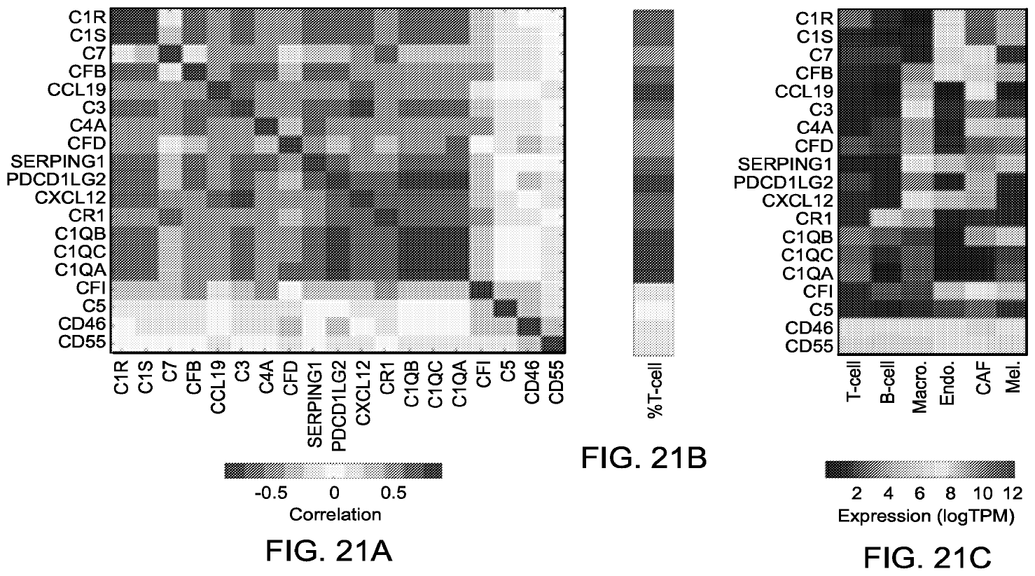

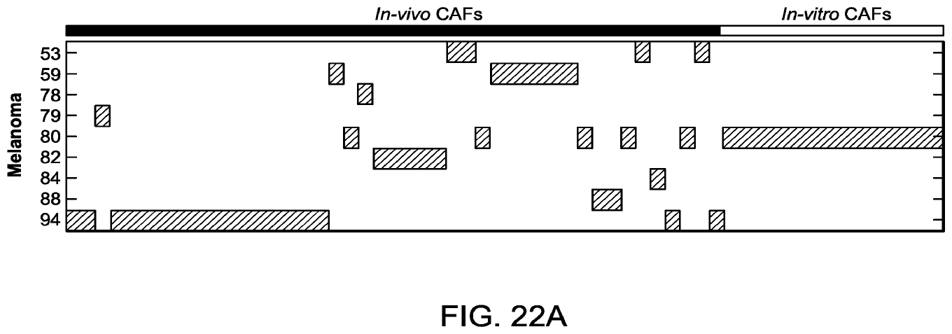

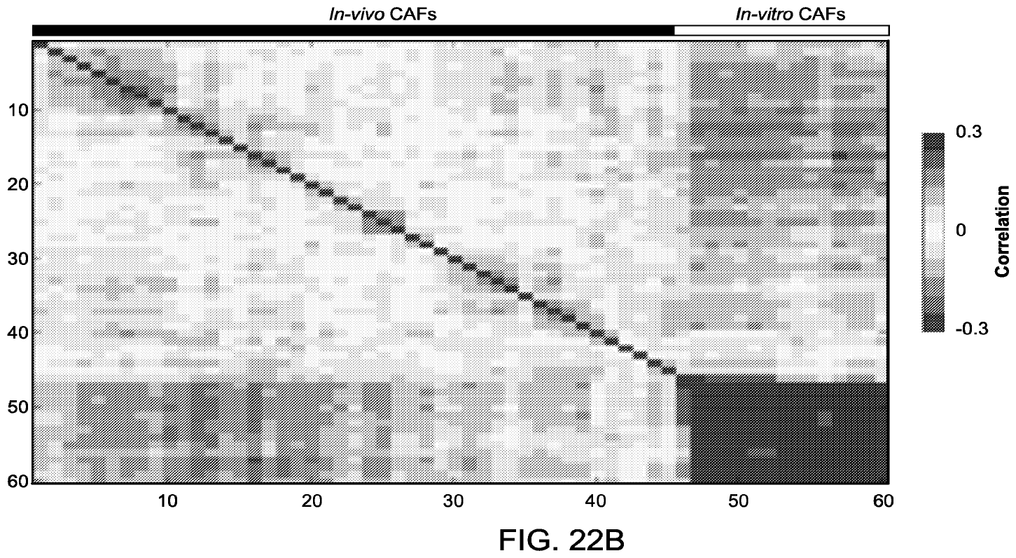

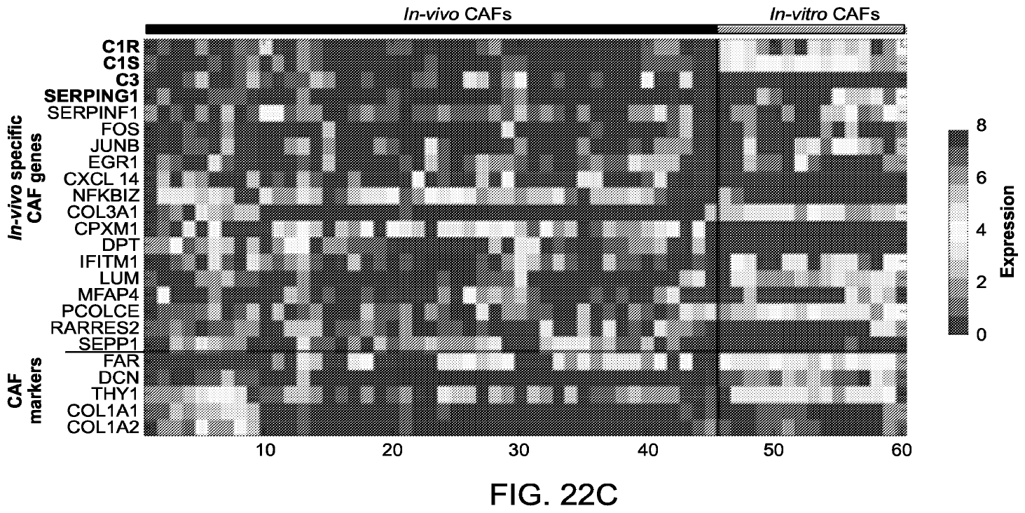

Interactions between cells play crucial roles in the tumor microenvironment. To assess systematically how cell-cell interactions may influence tumor composition, genes expressed by cells of one type that may influence the proportion of cells of a different type in the tumor were considered (FIG. 20A-FIG. 20C). For example, genes expressed primarily by CAFs (but not T cells) in single cell data that correlated with T cell abundance (as inferred by T cell specific genes) were searched in bulk tumor tissue from the TCGA data set (Akbani et al., 2015 Cell., 161: 1681-1696). A set of CAF-expressed genes was identified that correlated strongly with T cell infiltration (FIG. 4B, red circles). These included known chemotactic (CXCL12, CCL19) and immune modulating (PD-L2) genes, which are significantly expressed by both CAFs and macrophages (FIG. 21A-FIG. 21C). A separate set of genes exclusively expressed by CAFs that correlated with T cell infiltration (FIG. 21A-FIG. 21C) included multiple complement factors (C1S, CJR, C3, C4A, CFB and C1NH [SERPING1]). Notably, these complement genes were specifically expressed by freshly isolated CAFs but not by cultured CAFs (FIG. 22A-FIG. 22C) or macrophages (FIG. 21A-FIG. 21C). These findings are intriguing in light of several studies that have implicated complement activity in the recruitment and modulation of T cell mediated anti-tumor immune responses (in addition to the established role of complement in innate immunity (Markiewski et al., 2008 Nat. Immunol., 9: 1225-1235).

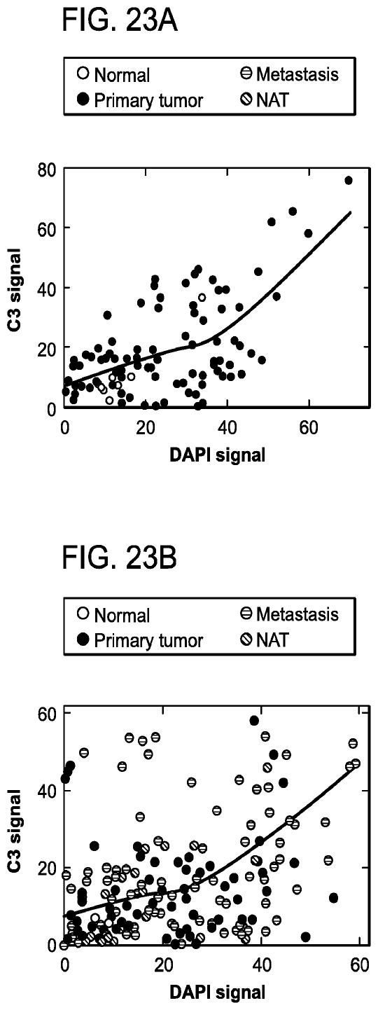

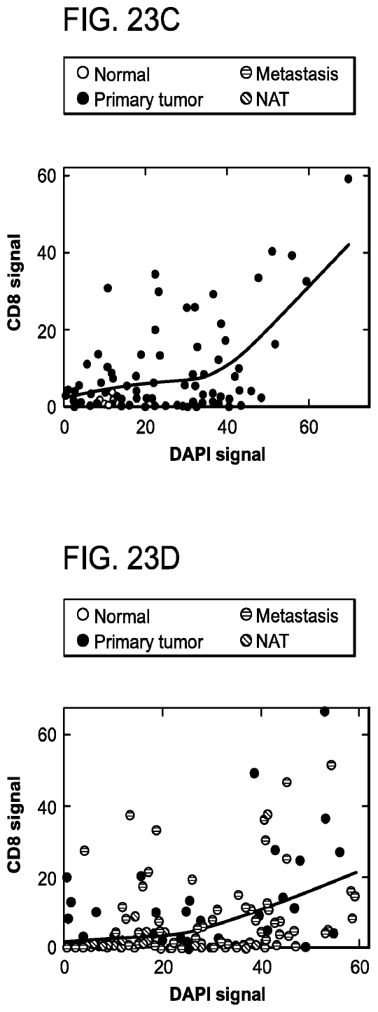

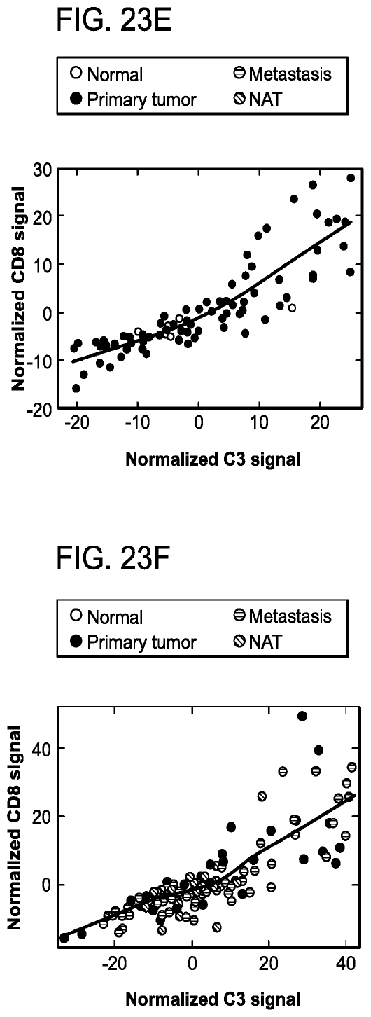

A high correlation (R>0.8) between complement factor 3 (C3) levels (one of the CAF-expressed complement genes) and infiltration of CD8+ T cells was validated. To this end, dual IF staining and quantitative slide analysis of two tissue microarrays (TMAs) was performed with a total of 308 core biopsies, including primary tumors, metastatic lesions, normal skin with adjacent tumor and healthy skin controls (FIG. 4C; FIG. 23A-FIG. 23F, Example 1). To test the generalizability of the association between CAF-derived complement factors with T cell infiltration, the analysis was expanded to bulk RNA-seq datasets across all TCGA cancer types (FIG. 4D). Consistent with the results in melanoma, complement factors correlated with inferred T cell abundance in many cancer types, and more highly than in normal tissues (e.g., R>0.4 for 65% of cancer types but only for 14% of normal tissue types). Although correlation analysis cannot determine causality, this indicates a potential in vivo role for cell-to-cell interactions.

### Example 7: Diversity of Tumor-Infiltrating T Lymphocytes and their Functional States

The activity of tumor-infiltrating lymphocytes (TILs)—in particular CD8+ T cells—is a major determinant of successful immune surveillance. Under normal circumstances, effector CD8+ T cells exposed to antigens and co-stimulatory factors mediate lysis of malignant cells and control tumor growth. However, this function can be hampered by tumor-mediated T cell exhaustion, such that T cells fail to activate cytotoxic effector functions (E. J. Wherry, 2011 Nat. Immunol., 12: 492-499). Exhaustion is promoted through the stimulation of coinhibitory “checkpoint” molecules on the T cell surface (PD-1, TIM-3, CTLA-4, TIGIT, LAG3 and others) (L. Chen and D. B. Flies, 2013 Nat. Rev. Immunol., 13: 227-242); blockade of checkpoint mechanisms has shown remarkable clinical benefit in subsets of melanoma and other malignancies (Hodi et al., 2010 N. Engl. J. Med., 363: 711-723; Larkin et al., 2015 N. Engl. J. Med., 373: 23-34; Borghaei et al., 2015 N. Engl. J. Med., 373: 1627-1639; Motzer et al., 2015 N. Engl. J. Med., 373: 1803-1813). While checkpoint ligand expression (e.g., PD-L1) and neoantigen load clearly contribute (K. M. Mahoney and M. B. Atkins, 2014 Oncol. Williston Park N. 28, Suppl 3, 39-48; Rizvi et al., 2015 Science, 348: 124-128; Van Allen et al., 2015 Science, 350: 207-211), prior to the invention described herein, no biomarker has emerged that reliably predicts the clinical response to immune checkpoint blockade. As described herein, single cell analyses yield features that can be used in the future to elucidate response determinants and possibly identify new immunotherapy targets.

To characterize this diversity in human tumors, the single-cell expression patterns of 2,068 T cells from 15 melanomas were analyzed. T cells and their main subsets (CD4+, Tregs, and CD8+) were identified based on the expression levels of their respective defining surface markers (FIG. 5A, top and Table 11).

TABLE 11
List of genes preferentially expressed in Tregs
compared to CD4+ and CD8+ T-cells.

| | Tregs/CD4+ | | Tregs/CD8+ | |
|---|---|---|---|---|
| | | significance | | significance |
| Gene Name | log2-ratio | (−log10(P)) | log2-ratio | (−log10(P)) |
| IL2RA | 4.9314 | 108.0864 | 4.9429 | 156.3565 |
| FOXP3 | 4.203 | 89.2082 | 4.3284 | 196.1143 |
| S100A4 | 3.4739 | 10.3922 | 3.6712 | 12.825 |
| CCR8 | 3.4462 | 34.0957 | 3.6126 | 100.6657 |
| TNFRSF1B | 3.3038 | 14.9444 | 2.4584 | 9.0528 |
| GBP5 | 3.2691 | 21.9609 | 1.994 | 7.2986 |
| TNFRSF18 | 3.1395 | 13.1937 | 3.8084 | 39.3184 |
| IFI6 | 3.1378 | 10.4917 | 2.4915 | 7.0957 |
| CXCR6 | 2.8035 | 11.1341 | 1.2444 | 1.8837 |
| PIM2 | 2.783 | 9.7392 | 3.6418 | 19.0767 |
| LGALS1 | 2.7658 | 10.2398 | 2.1396 | 6.2732 |
| BATF | 2.7427 | 8.9412 | 2.9111 | 11.5239 |
| TNFRSF4 | 2.7405 | 11.0809 | 3.724 | 67.4286 |
| GBP2 | 2.6039 | 8.5013 | 2.0545 | 5.6399 |
| S100A6 | 2.4478 | 7.2581 | 1.853 | 4.9506 |
| UGP2 | 2.4448 | 9.5419 | 2.6079 | 12.8918 |
| CTSC | 2.4278 | 14.0409 | 2.1092 | 10.6288 |
| SAT1 | 2.411 | 6.4101 | 2.5169 | 7.0602 |
| IL32 | 2.4067 | 10.6603 | 2.0114 | 10.4194 |
| APOBEC3C | 2.384 | 6.8456 | 0.3962 | 0.3762 |
| IL2RB | 2.3507 | 10.0447 | 1.3959 | 4.1239 |
| CTLA4 | 2.2923 | 8.1621 | 2.226 | 9.679 |
| ENO1 | 2.2681 | 6.577 | 2.6227 | 8.4014 |
| ACP5 | 2.2576 | 8.6929 | 1.5582 | 3.7963 |
| SELPLG | 2.2563 | 6.2061 | 2.5352 | 8.7096 |
| COX17 | 2.2174 | 10.9203 | 1.8901 | 7.6237 |
| CCND2 | 2.1527 | 10.5771 | 1.3008 | 3.7349 |
| PRDX3 | 2.1424 | 8.6678 | 1.4985 | 3.8471 |
| LAIR2 | 2.1415 | 13.851 | 2.0799 | 15.8578 |
| LTB | 2.1273 | 4.2022 | 4.7733 | 34.5617 |
| PRDM1 | 2.1105 | 8.2645 | 1.4024 | 4.2404 |
| HSPA1A | 2.0835 | 5.9936 | −0.2198 | 0.1588 |
| IL10RA | 2.0721 | 5.9976 | 1.1443 | 2.1226 |
| PRNP | 2.0648 | 6.5277 | 2.5922 | 13.0264 |
| TYMP | 2.0431 | 15.7423 | 1.5948 | 7.2617 |
| NDUFA13 | 2.0129 | 5.016 | 1.8961 | 4.5219 |
| SYNGR2 | 1.9999 | 5.7351 | 1.3058 | 2.5734 |
| SQSTM1 | 1.9941 | 7.2362 | 1.6929 | 5.4276 |
| STAT1 | 1.9898 | 4.858 | 1.733 | 3.7968 |
| LINC00152 | 1.9851 | 6.3335 | 0.9553 | 1.7154 |
| CD27 | 1.9849 | 4.1972 | 0.6058 | 0.7365 |
| CXCR3 | 1.98 | 5.3375 | 1.6348 | 4.0588 |
| TIGIT | 1.9668 | 4.6304 | 0.6416 | 0.8306 |
| MRPS6 | 1.9596 | 6.3062 | 1.9272 | 6.9118 |
| CLIC1 | 1.9249 | 4.5393 | 1.2696 | 2.3622 |
| PARK7 | 1.9208 | 4.2626 | 1.2864 | 2.1789 |
| CD74 | 1.92 | 4.7128 | −0.1704 | 0.202 |
| SDC4 | 1.8928 | 17.7383 | 1.775 | 16.7533 |
| SOD1 | 1.8784 | 4.6144 | 1.5636 | 3.4375 |
| FTL | 1.8447 | 5.5337 | 1.0957 | 2.5111 |
| ISG15 | 1.8244 | 3.5101 | 1.4318 | 2.4338 |
| LY6E | 1.7697 | 4.5628 | 1.3713 | 3.0396 |
| DUSP4 | 1.7572 | 5.7029 | −0.1149 | 0.1174 |
| GCHFR | 1.7485 | 7.5737 | 1.5724 | 6.2974 |
| TPM4 | 1.7445 | 4.8499 | 2.1814 | 8.719 |
| PRF1 | 1.7444 | 6.3169 | −2.1843 | 5.3341 |
| ACTN4 | 1.7392 | 7.4175 | 0.7837 | 1.5797 |
| ANKRD10 | 1.7306 | 5.9561 | 1.4854 | 4.7378 |
| FAM110A | 1.7248 | 8.838 | 1.7443 | 11.1629 |
| COX5A | 1.7214 | 4.2827 | 1.5293 | 3.3323 |
| CST7 | 1.6971 | 3.5333 | −2.2012 | 6.2886 |
| GABARAP | 1.691 | 4.0968 | 1.6808 | 4.0383 |
| PHLDA1 | 1.6828 | 11.0367 | 0.9662 | 3.0102 |
| SUMO2 | 1.6769 | 3.9712 | 1.8155 | 4.5819 |
| TAP1 | 1.6768 | 3.7399 | 0.6796 | 0.921 |
| VCP | 1.6724 | 4.3504 | 1.7534 | 5.0804 |
| ICOS | 1.6511 | 3.1124 | 2.5341 | 8.9582 |
| C17orf49 | 1.6435 | 4.1573 | 1.2955 | 2.595 |
| IL2RG | 1.6364 | 3.9312 | 1.4064 | 3.0846 |
| BUB3 | 1.6249 | 3.8154 | 0.8231 | 1.2816 |
| PEBP1 | 1.5804 | 3.3888 | 1.6761 | 4.1517 |
| PLP2 | 1.5799 | 3.9804 | 1.4823 | 3.7429 |
| LSP1 | 1.5742 | 3.1647 | 0.6289 | 0.8449 |
| NAMPT | 1.5693 | 7.2891 | 1.7405 | 11.5589 |
| CRADD | 1.5687 | 11.3383 | 1.6363 | 20.1184 |
| ATP6V0E1 | 1.567 | 3.0378 | 1.8802 | 4.0639 |
| PRDX6 | 1.562 | 4.886 | 1.1606 | 2.7899 |
| SPPL2A | 1.5464 | 4.9576 | 1.4549 | 4.7904 |
| PSMB3 | 1.5383 | 2.8248 | 1.2727 | 2.1416 |
| BST2 | 1.5219 | 3.6094 | 1.0841 | 1.9052 |
| SLAMF1 | 1.5193 | 4.5894 | 2.282 | 19.8918 |
| CRIP1 | 1.5172 | 2.6247 | 0.9933 | 1.423 |
| CSF1 | 1.507 | 9.8658 | 0.8546 | 2.475 |
| DUSP16 | 1.5059 | 8.837 | 1.4756 | 10.197 |
| LGALS3 | 1.5045 | 4.0982 | 1.4202 | 4.2955 |
| OTUB1 | 1.4974 | 4.3779 | 1.584 | 4.9134 |
| PDIA6 | 1.4971 | 4.0511 | 0.7905 | 1.2344 |
| GABARAPL2 | 1.491 | 3.595 | 1.4439 | 3.4709 |
| GLRX | 1.4862 | 3.8439 | 1.8348 | 6.5624 |
| CD7 | 1.4846 | 6.6389 | 0.4425 | 0.7692 |
| IL1R2 | 1.4826 | 12.7171 | 1.554 | 35.0035 |
| TPI1 | 1.4791 | 2.4408 | 0.8294 | 1.0138 |
| MX1 | 1.4784 | 5.0034 | 1.1599 | 3.1162 |
| PBXIP1 | 1.4711 | 4.141 | 2.8843 | 20.6602 |
| HLA- | 1.4666 | 3.4947 | −1.4391 | 2.5483 |
| DPA1 | | | | |
| OAS1 | 1.464 | 5.6234 | 1.3653 | 5.4415 |
| FBXW5 | 1.4636 | 4.5146 | 1.5089 | 5.6328 |
| ANXA2 | 1.4608 | 2.6396 | 1.3945 | 2.6863 |
| RTKN2 | 1.4583 | 18.869 | 1.5568 | 51.7679 |
| LASP1 | 1.4533 | 4.1449 | 1.2308 | 3.2262 |
| TNFRSF9 | 1.4497 | 11.6612 | −0.1722 | 0.2282 |
| WDR1 | 1.448 | 3.6362 | 1.4179 | 3.6517 |
| SH2D2A | 1.4454 | 4.9413 | 0.9791 | 2.4114 |
| MYL6 | 1.4434 | 4.2888 | 1.3482 | 3.5196 |
| ACAA1 | 1.4389 | 4.0391 | 1.5627 | 5.6314 |
| NOP10 | 1.4334 | 3.3827 | 1.078 | 2.0201 |
| DPYSL2 | 1.4279 | 8.1775 | 1.477 | 11.114 |
| PSMD2 | 1.4239 | 4.1145 | 1.25 | 3.3147 |
| CCR5 | 1.4169 | 4.3057 | 0.3008 | 0.3365 |
| HAPLN3 | 1.4067 | 4.509 | 1.6356 | 7.8559 |
| COX6B1 | 1.3985 | 2.9477 | 1.304 | 2.7498 |
| MYO1G | 1.3971 | 4.5973 | 0.7691 | 1.4872 |
| CTSA | 1.3948 | 3.7213 | 1.5284 | 4.7298 |
| CALM3 | 1.3864 | 4.6899 | 0.9947 | 2.6976 |
| PTPN7 | 1.3846 | 3.1375 | 0.707 | 1.0896 |
| CTNNB1 | 1.3846 | 4.5104 | 1.1333 | 3.2912 |
| PHTF2 | 1.384 | 4.0246 | 2.2315 | 14.1826 |
| PSMB1 | 1.3829 | 2.2889 | 1.7349 | 3.5906 |
| ATP5B | 1.3802 | 2.4225 | 1.4684 | 2.7511 |
| ARRDC1 | 1.371 | 4.1943 | 1.2726 | 3.7427 |
| PTTG1 | 1.3517 | 3.4075 | 1.2953 | 3.4109 |
| TPP1 | 1.3507 | 3.2258 | 1.8232 | 6.3944 |
| ISG20 | 1.3489 | 2.5137 | 1.2107 | 2.0813 |
| TWF2 | 1.3486 | 3.2437 | 1.1262 | 2.3436 |
| EID1 | 1.3459 | 3.2424 | 0.9325 | 1.7275 |
| ATP5E | 1.3441 | 2.8331 | 0.6234 | 1.0373 |
| ARPC1B | 1.3416 | 2.5386 | 1.8015 | 4.0743 |
| NDUFB8 | 1.3414 | 2.4351 | 0.8999 | 1.294 |
| SHMT2 | 1.3395 | 4.7184 | 1.4804 | 7.3149 |
| TUBB | 1.3374 | 2.4108 | 1.0608 | 1.6405 |
| HLA- | 1.3265 | 3.3234 | −1.6063 | 3.6511 |
| DRB1 | | | | |
| DDB2 | 1.3116 | 4.3634 | 1.416 | 5.6489 |
| TANK | 1.3091 | 3.1295 | 1.2604 | 3.0242 |
| NCF4 | 1.3041 | 4.484 | 1.8421 | 21.6217 |
| TMEM60 | 1.2997 | 5.1834 | 1.3407 | 7.5323 |
| PSMA1 | 1.2991 | 2.5203 | 1.4163 | 3.0406 |
| TCEB2 | 1.293 | 3.1752 | 1.2509 | 3.0595 |
| APOBEC3G | 1.2918 | 2.9403 | −1.118 | 1.7578 |
| ARHGAP9 | 1.2876 | 3.1194 | 0.8446 | 1.5337 |
| SERPINB9 | 1.2814 | 3.5861 | 0.5383 | 0.8663 |
| CMC2 | 1.2791 | 3.325 | 1.2574 | 3.3681 |
| WSB1 | 1.2712 | 3.8498 | 1.1098 | 3.0142 |
| PLD3 | 1.2689 | 5.2576 | 1.264 | 5.76 |
| GPS2 | 1.2629 | 2.9045 | 1.2236 | 3.0433 |
| OCIAD2 | 1.2578 | 2.444 | 1.6864 | 4.5153 |
| SNX5 | 1.2562 | 3.7595 | 1.248 | 3.7184 |
| DGUOK | 1.2562 | 3.185 | 1.2082 | 3.1996 |
| IKZF2 | 1.2556 | 10.2888 | 1.1321 | 9.9732 |
| GPX1 | 1.2503 | 2.278 | 2.0277 | 7.8061 |
| PTPN1 | 1.25 | 4.3921 | 1.1973 | 4.4626 |
| VDR | 1.2404 | 9.2804 | 1.1793 | 9.6917 |
| SAMD9 | 1.2355 | 6.636 | 0.8628 | 2.9563 |
| RAC2 | 1.2345 | 2.4824 | 1.2087 | 2.4981 |
| RPS27L | 1.2258 | 3.8407 | 1.4026 | 5.5632 |
| EPS15 | 1.2232 | 4.1322 | 1.1412 | 3.9182 |
| CAP1 | 1.2229 | 2.6631 | 1.2053 | 2.6106 |
| AP2M1 | 1.2219 | 2.5587 | 1.0708 | 2.1636 |
| NDUFB10 | 1.2218 | 2.5617 | 0.9597 | 1.6679 |
| AGTRAP | 1.2206 | 4.0087 | 1.2162 | 4.5654 |
| IRF9 | 1.2192 | 2.3886 | 0.5484 | 0.6954 |
| HLA-DMA | 1.2021 | 4.5233 | −0.7323 | 1.0207 |
| MAGEH1 | 1.1986 | 2.9482 | 1.7923 | 11.8359 |
| TMED9 | 1.1941 | 2.2484 | 1.3405 | 3.0532 |
| TFRC | 1.1938 | 4.0512 | 1.1977 | 4.2677 |
| EMP3 | 1.1936 | 2.3379 | 1.5454 | 3.9512 |
| RHOF | 1.1931 | 2.8382 | 1.3896 | 3.8433 |
| PGK1 | 1.193 | 2.1025 | 1.0509 | 1.8193 |
| CAST | 1.1865 | 4.0358 | 1.2894 | 5.0711 |
| CD58 | 1.1837 | 2.8965 | 1.2941 | 3.6738 |
| NDUFV2 | 1.1791 | 2.0201 | 1.5293 | 3.417 |
| CD79B | 1.1785 | 3.4684 | 1.3654 | 5.5062 |
| PAIP2 | 1.1768 | 2.1353 | 1.0782 | 1.8948 |
| TARDBP | 1.1747 | 3.3346 | 1.0885 | 2.9811 |
| SFT2D1 | 1.1747 | 2.5526 | 0.8662 | 1.5283 |
| STAM | 1.1737 | 4.6628 | 1.491 | 11.2261 |
| GBP4 | 1.1683 | 5.7353 | 0.759 | 2.3531 |
| HPRT1 | 1.1606 | 4.0411 | 0.9824 | 2.8081 |
| TMSB10 | 1.1575 | 5.6919 | 1.2878 | 6.425 |
| U2AF1L4 | 1.1552 | 3.9465 | 0.9408 | 2.7047 |
| TPM3 | 1.1527 | 3.6936 | 1.2356 | 4.1502 |
| C3AR1 | 1.1519 | 8.6292 | 1.1896 | 14.5168 |
| CDKN1B | 1.1507 | 2.8125 | 0.7531 | 1.3981 |
| TMEM173 | 1.1454 | 2.149 | 1.802 | 5.8798 |
| TRAPPC1 | 1.1423 | 3.2075 | 1.1024 | 3.1881 |
| RAP1A | 1.1422 | 2.9078 | 1.2535 | 3.847 |
| NFKBIZ | 1.1405 | 2.7426 | 1.6435 | 6.4682 |
| HERPUD1 | 1.1375 | 2.1122 | 0.8367 | 1.3027 |
| FKBP1A | 1.1366 | 2.1013 | 0.8428 | 1.3552 |
| B4GALT1 | 1.1362 | 3.546 | 1.2567 | 4.9898 |
| EIF4A1 | 1.1359 | 2.0004 | 1.271 | 2.4293 |
| OTUD5 | 1.1356 | 4.8059 | 1.2142 | 6.3012 |
| IRF2 | 1.1321 | 3.5988 | 0.3738 | 0.5464 |
| CCR4 | 1.1316 | 2.2499 | 2.2758 | 23.2853 |
| RHOC | 1.1306 | 3.0064 | 0.7756 | 1.5918 |
| ADORA2A | 1.1301 | 4.2427 | 0.6748 | 1.3801 |
| MRPL36 | 1.1285 | 4.8562 | 0.9545 | 3.3227 |
| PMAIP1 | 1.1283 | 3.3635 | 0.4399 | 0.6228 |
| RNF213 | 1.1278 | 5.5662 | 0.7493 | 3.1218 |
| REREP3 | 1.1263 | 4.3411 | 1.5126 | 23.4758 |
| ARPC5L | 1.1254 | 2.565 | 0.5489 | 0.7658 |
| VDAC2 | 1.123 | 2.2417 | 1.1622 | 2.5702 |
| HSD17B10 | 1.1222 | 2.5763 | 1.311 | 4.0266 |
| PELI1 | 1.1215 | 3.9849 | 1.3548 | 7.7508 |
| MRPS7 | 1.1196 | 2.974 | 1.076 | 2.9395 |
| GNPTAB | 1.1181 | 6.5425 | 0.9386 | 4.3756 |
| YWHAE | 1.1092 | 2.9974 | 0.689 | 1.253 |
| ATP6V1E1 | 1.1076 | 2.5331 | 0.9287 | 1.9102 |
| GALM | 1.107 | 3.0304 | 0.7437 | 1.4177 |
| ERI1 | 1.1069 | 7.1931 | 1.2037 | 11.6122 |
| BANF1 | 1.1031 | 3.3315 | 0.8063 | 1.8427 |
| SAMSN1 | 1.102 | 2.2355 | 1.2736 | 3.134 |
| TXN | 1.1018 | 2.8026 | 1.0062 | 2.5035 |
| PRDX5 | 1.0999 | 2.0767 | 0.5756 | 0.7511 |
| PIP4K2C | 1.0991 | 3.5209 | 1.1964 | 4.7433 |
| CMTM7 | 1.096 | 2.2708 | 1.4967 | 5.2249 |
| FCRL3 | 1.0957 | 4.8266 | −0.8363 | 1.463 |
| COX7A2L | 1.0953 | 2.0561 | 1.2282 | 2.7693 |
| GNG5 | 1.0911 | 2.0219 | 0.9472 | 1.7154 |
| ACTR1A | 1.0874 | 3.2474 | 1.0875 | 3.6302 |
| APLP2 | 1.0855 | 3.9035 | 0.9113 | 3.0437 |
| CSF2RB | 1.0854 | 11.8913 | 1.1409 | 33.281 |
| EXOSC7 | 1.0825 | 3.6053 | 1.0241 | 3.4395 |
| CACYBP | 1.082 | 2.974 | 0.717 | 1.4253 |
| PPP2R1A | 1.0791 | 2.1016 | 1.0792 | 2.1817 |
| MGAT1 | 1.0713 | 2.5957 | 0.8291 | 1.6717 |
| OVCA2 | 1.0697 | 2.9705 | 0.8743 | 2.0155 |
| UBA1 | 1.069 | 2.4156 | 1.2125 | 3.1312 |
| REC8 | 1.0664 | 5.4073 | 0.9344 | 4.2368 |
| KCNN4 | 1.0573 | 5.442 | 0.9763 | 4.7937 |
| ARHGEF6 | 1.0563 | 2.734 | 1.6628 | 8.1901 |
| RFK | 1.0544 | 5.8307 | 1.126 | 11.0342 |
| HTATIP2 | 1.0401 | 3.723 | 0.8485 | 2.3564 |
| ANXA11 | 1.0358 | 2.3683 | 1.0522 | 2.5286 |
| MAPKAPK3 | 1.0335 | 3.269 | 1.1717 | 5.0343 |
| SNX10 | 1.0335 | 6.1494 | 0.9935 | 6.6335 |
| PSMA5 | 1.0241 | 2.7636 | 0.9663 | 2.4943 |
| BIRC3 | 1.0224 | 2.5934 | 1.3975 | 5.2056 |
| NDUFA3 | 1.0207 | 2.2145 | 0.7994 | 1.5508 |
| GATA3 | 1.017 | 3.9346 | 1.0305 | 4.1607 |
| SDF4 | 1.0169 | 2.6697 | 1.3371 | 5.3809 |
| UBE2B | 1.0132 | 2.8088 | 1.0963 | 3.5892 |
| NEMF | 1.013 | 3.287 | 0.8904 | 2.6344 |
| NDUFA11 | 1.002 | 2.1448 | 0.8833 | 1.7486 |
| SDF2L1 | 1.002 | 2.9401 | 0.7455 | 1.6546 |

In Table 11, all genes were significantly higher expressed (P<0.01, fold-change>2) in Tregs compared to other CD4+ T-cells. Genes were sorted by fold-change increase in T-regs compared to other CD4+ T-cells, as shown in the second column of Table 11. Fourth and fifth columns of Table 11 contain the log-ratio and p-value in comparison of Tregs to CD8+ T-cells; this comparison was not used to define the gene-list, but is provided as additional information.

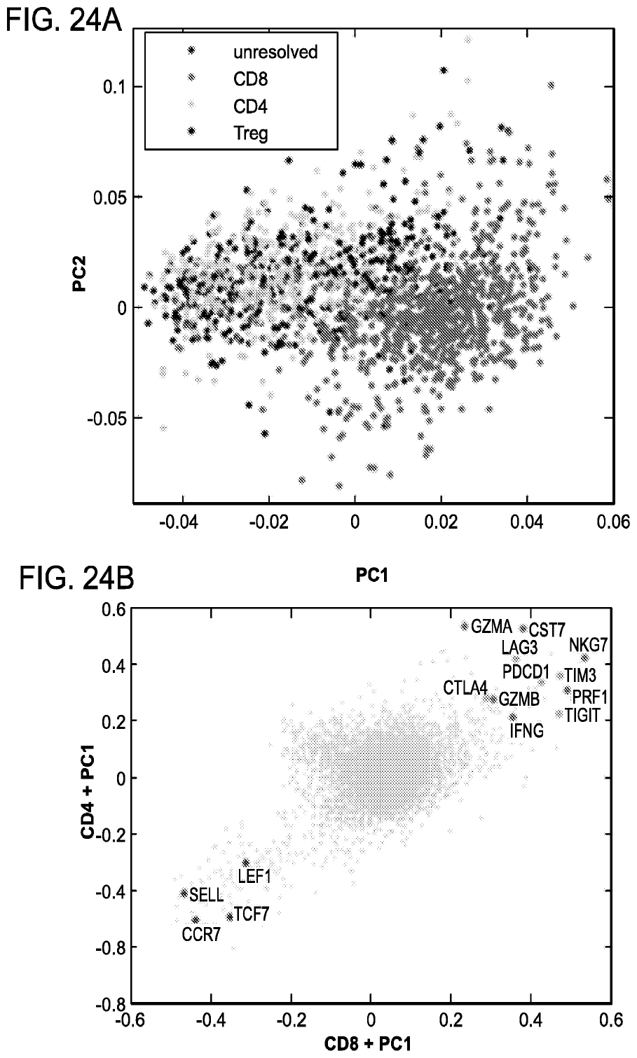

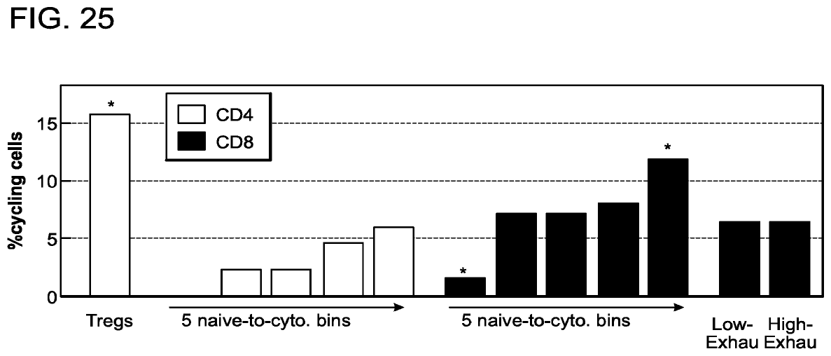

Within both the CD4+ and CD8+ populations, a principal component analysis distinguished cell subsets and heterogeneity of activation states based on expression of naïve and cytotoxic T cell genes (FIG. 5A-FIG. 5B and FIG. 24A-FIG. 24B). Next, the exhaustion status of each cell was determined, based on the expression of key coinhibitory receptors (PD1, TIGIT, TIM3, LAGS and CTLA4). In several cases, these co-inhibitory receptors were co-expressed across individual cells; this phenomenon was validated for PD1 and TIM3 by immunofluorescence (FIG. 5C). However, exhaustion gene expression was also highly correlated with the expression of both cytotoxicity markers and overall T cell activation states (FIG. 5B). This observation resembles an “activation-dependent exhaustion expression program” previously reported in models of chronic viral infections (E. J. Wherry et al., 2007 Immunity, 27: 670-684). Accordingly, expression of co-inhibitory receptors (alone or in combinations) per se may not be sufficient to characterize the salient functional state of tumor associated T lymphocytes in situ or to distinguish exhaustion from activation.

To define an “activation-independent exhaustion program”, single-cell data was leveraged from a large number of CD8+ T cells sequenced in a single tumor (Mel75, 314 cells). These data allowed tumor cytotoxic and exhaustion programs to be deconvolved. Specifically, PCA of Mel75 T cell transcriptomes identified a robust expression module that consisted of all five co-inhibitory receptors and other exhaustion-related genes, but not cytotoxicity genes (FIG. 26A-FIG. 26B and Table 12).

The Mel75 exhaustion program was used, as well as two previously published exhaustion programs (E. J. Wherry et al., 2007 Immunity, 27: 670-684; Baitsch et al., 2011 J. Clin. Invest., 121: 2350-2360) to estimate the exhaustion state of each cell. Here, exhaustion state was defined as “high” or “low” expression of the exhaustion program relative to that of cytotoxicity genes (FIG. 5D, Example 1). Accordingly, exhaustion states were defined in Mel75 and in four additional tumors with the highest number of CD8+ T cells (68 to 214 cells per tumor). The top genes that were preferentially expressed in high-exhaustion compared to low-exhaustion cells (both defined relative to the expression of cytotoxicity genes) were identified. Finally, a core exhaustion signature was defined across cells from various tumors.

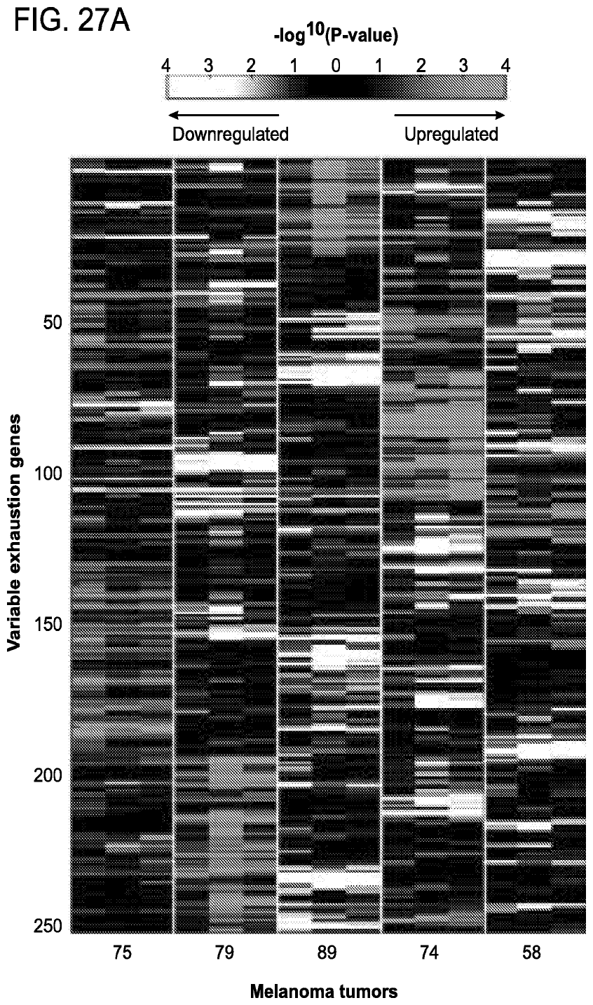

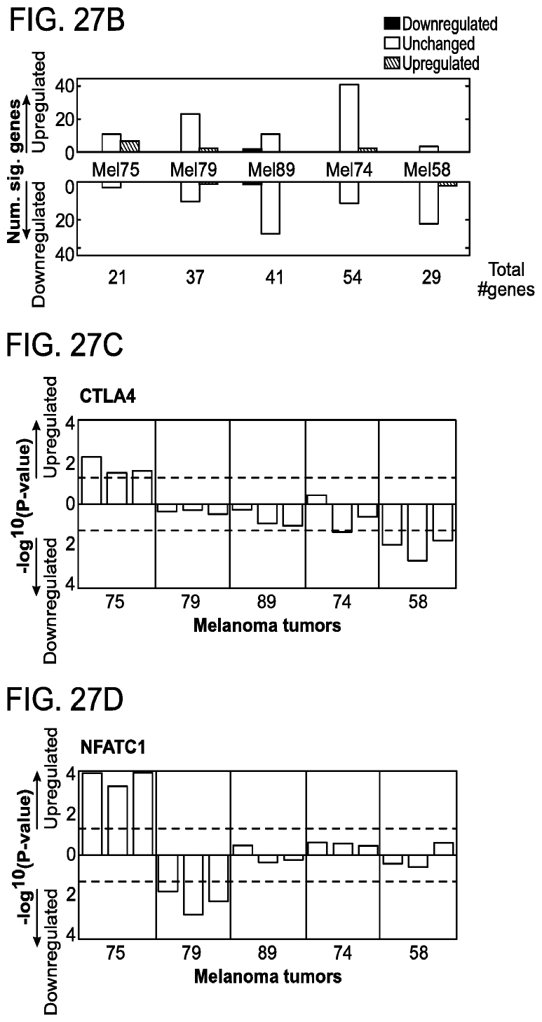

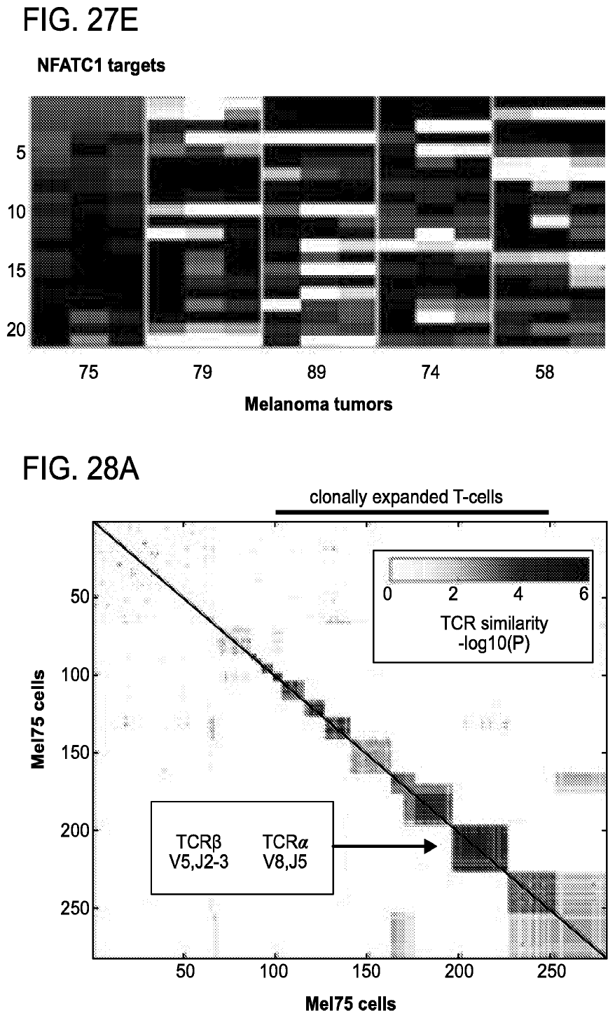

Substantial variation was observed between patients in the high exhausted cells, which may mirror the variation in treatment responses or history. Nonetheless, the core exhaustion signature yielded 28 genes that were consistently upregulated in high-exhaustion cells of most tumors, including co-inhibitory (TIGIT) and co-stimulatory (TNFRSF9/4-1BB, CD27) receptors (FIG. 5E). In addition, most genes that were significantly upregulated in high-exhaustion cells of at least one tumor had distinct associations with exhaustion across the different tumors (FIG. 5F, 272 of 300 genes with P<0.001 by permutation test; FIG. 27A-B). These tumor-specific signatures included variable expression of known exhaustion markers (Table 12), and could be linked to response to immunotherapies or reflect the effects of previous treatments. For example, CTLA4 was highly upregulated in exhausted cells of Mel75 and weakly upregulated in three other tumors, but was completely decoupled from exhaustion in Mel58. Interestingly, Mel58 was derived from a patient with initial response and subsequent development of resistance to CTLA-4 blockade with ipilimumab (FIG. 5F, FIG. 27C). Another variable gene of interest was the transcription factor NFATC1, which was previously implicated in T cell exhaustion (Martinez et al., 2015 Immunity, 42: 265-278). NFATC1 and its target genes were strongly associated with the activation-independent exhaustion phenotype in Mel75 (FIG. 27D-Fi. 27E), suggesting a potential role of NFATC1 in the underlying variability of exhaustion programs among patients.

Finally, the relationship between T cell states and clonal expansion was explored. T cells that recognize tumor antigens may proliferate to generate discernible clonal subpopulations defined by an identical T cell receptor (TCR) sequence (Blackburn et al., 2008 Proc. Natl. Acad. Sci. U.S.A., 105(39): 15016-21). To identify potential expanded T cell clones, RNA-seq reads that map to the TCR were used to classify single T cells by their isoforms of the V and J segments of the alpha and beta TCR chains, and enriched combinations of TCR segments were searched. Most observed combinations were found in few cells and were not enriched. However, approximately half of the CD8+ T cells in Mel75 had one of seven enriched combinations identified (FDR=0.005), and thus may represent expanded T cell clones (FIG. 5G, FIG. 28A-FIG. 28B). Interestingly, this putative T cell expansion was also linked to exhaustion (FIG. 5H), such that low-exhaustion T cells were significantly depleted of expanded T cells (TCR clusters with >6 cells) and enriched in non-expanded T cells (TCR clusters with 1-4 cells). In particular, the non-exhausted cytotoxic cells are almost all non-expanded (FIG. 4H). As described herein, single-cell RNA-seq profiling of T cells derived from patient tumors before and after treatment with immune checkpoint inhibitors directly measure the dynamics of clonal and functional architecture and their associated treatment outcomes. Overall, this analysis suggests that single-cell RNA-seq allows inference of functionally variable T cell populations that are not detectable with other profiling approaches (FIG. 29). This knowledge empowers studies of tumor response and resistance to immune checkpoint inhibitors.

### Example 8: Dissociation of Tumor for Genomic Analysis

Described in detail below are optimizations of the protocols described herein.

### Breast Cancer Samples

Seven (7) patient-derived samples were processed using different protocols to optimize the process of cellular dissociation. Samples 301 and 306 were treated using the protocol described in Tirosh et al., 2016 Science, 352(6282): 189-196 reproduced below:
1. Tumor is transported to the lab in PBS and on ice;
2. Cut sample tissue into tiny cubes (1×1 mm³) using scalpels;
3. Make digestion buffer—remove 5 ml M199 medium from 37° C. water bath and add collagenase P (2 mg/ml) and DNase I (10 μg/μl);
4. Using scalpels, transport tumor cubes to media in a 15 ml Falcon tube, use 1 ml digestion buffer to wash off cells from dish and add to the 15 ml tube;
5. Place 15 ml tube in 37° C. water bath for 10 min;
6. Remove tube, vortex on maximum speed for 10 seconds;
7. Use 5 ml followed by 2 ml pipette to pipette up and down (at least 10 times) and repeat using 1000 μl pipette tip;
8. Repeat steps 6-7;
9. Place tube on ice. Using a 70 μm mesh, filter solution into new 15 ml Falcon tube;
10. Wash filter with 10 ml PBS+2% fetal calf serum (FCS);
11. Spin at 580G×5 minutes at 4° C., remove supernatant;
12. Resuspend in 2 ml PBS+2% FCS;
13. Staining protocol: prepare following tubes on ice:
   a. No stain control: 200 μl unstained cell solution;
   b. Calcein-AM: 200 μl cell solution+1 μl Calcein;
   c. CD45-FITC: 200 μl cell solution+1 μl CD45-FITC;
   d. EPCAM-PE: 200 μl cell solution+4 μl EPCAM-PE;
   e. Sample: 1200 μl cell solution+6 μl Calcein+6 μl CD45−+24 μl EPCAM;
14. Let calcein single color control and sample tube incubate at room temperature (RT) for 10-15 minutes, then place back on ice with other tubes;
15. Proceed to FACS, use 96-well plates containing 10 ul of lysis buffer (TCL buffer+1% beta Mercapto EtOH) in each well; sort viable cells (calcein positive) that are CD45 positive (immune cells) or CD45 negative and EpCam positive (cancer cells);
16. When sorting finished immediately seal the plate, vortex vigorously for 10 seconds, spin down at 3700 RPM for 2 minutes at 4° C. and place the plate on dry ice;
17. Store plates in −80° C. freezer.

The following patient samples were processed using modified protocols as identified below.

Sample 369 utilized a similar protocol, but the calcein viability staining was performed right before sorting, which aimed at higher viability representation. This protocol improved the quality of single cells from ˜5% to ˜20%.

Sample 376 was processed like the 369 protocol, with the addition of 7AAD reagent (dead cells staining) to ensure excluding dead cells at the onset of sorting. This improved the quality of single cells from ˜20% to >20% (CD45 negative cells).

Sample 386 was processed like the 376 protocol, but times of incubation and number of resuspensions were reduced by half. This improved the quality of single cells from <60% to ˜75% (CD45 positive cells).

Sample 398 was processed like the 386 protocol, but the filter and FACS nuzzle changed from 75 μm to 100 μm, digestion buffer volume—1:1 ratio [M199 buffer: collagenase IV (100 mg/ml)] and volume ratio 1:10 [M199 medium: 20 μl DNase I (1 μg/μl)]. This improved the quality of single cells from ˜20% to ˜25% (CD45 negative cells) and from ˜75% to 80% (CD45 positive cells).

Sample 467 was processed like the 398 protocol, but the enzymes were replaced with commercial reagent AccuMax (Innovative Cell Technologies, Inc., San Diego, Calif.) and calcein staining was discounted. This improved the quality of single cells from ˜25% to ˜45% (CD45 negative cells, usable cells) and from ˜80% to ˜85% (CD45 positive cells, useable cells). The results are shown in FIG. 33A-FIG. 33B.

The patient-derived samples varied between 1 core to 3 cores, yet no significant contribution was found to the number of passing single cells for 2 or 3 cores. Additional cores are a benefit for cellular quantity, while quality of single cells depends on additional factors, as listed above. The results for breast cancer samples are presented in FIG. 33A-FIG. 33B.

### Prostate Cancer Samples

First, the traditional protocol set forth below was applied as set forth below:
1. Tumor is transported to the lab in PBS and on ice;
2. Cut sample tissue into tiny cubes (1×1 mm³) using scalpels;
3. Make digestion buffer—remove 5 ml M199 medium from 37° C. water bath and add collagenase P (2 mg/ml) and DNase I (10 μg/μl);
4. Using scalpels, transport tumor cubes to media in a 15 ml Falcon tube, use 1 ml digestion buffer to wash off cells from dish and add to the 15 ml tube;
5. Place 15 ml tube in 37° C. water bath for 10 min;
6. Remove tube, vortex on maximum speed for 10 seconds;
7. Use 5 ml followed by 2 ml pipette to pipette up and down (at least 10 times) and repeat using 1000 μl pipette tip;
8. Repeat steps 6-7;
9. Place tube on ice. Using a 70 μm mesh, filter solution into new 15 ml Falcon tube;
10. Wash filter with 10 ml PBS+2% FCS;
11. Spin at 580G×5 minutes at 4° C., remove supernatant;
12. Resuspend in 2 ml PBS+2% FCS;
13. Staining protocol: prepare following tubes on ice:
   a. No stain control: 200 μl unstained cell solution;
   b. Calcein-AM: 200 μl cell solution+1 μl Calcein;
   c. CD45-FITC: 200 μl cell solution+1 μl CD45-FITC;
   d. EPCAM-PE: 200 μl cell solution+4 μl EPCAM-PE;
   e. Sample: 1200 μl cell solution+6 μl Calcein+6 μl CD45−+24 μl EPCAM;
14. Let calcein single color control and sample tube incubate at RT for 10-15 minutes, then place back on ice with other tubes;
15. Proceed to FACS, use 96-well plates containing 10 μl of lysis buffer (TCL buffer+1% beta Mercapto EtOH) in each well; sort viable cells (calcein positive) that are CD45 positive (immune cells) or CD45 negative and EpCam positive (cancer cells);
16. When sorting finished immediately seal the plate, vortex vigorously for 10 seconds, spin down at 3700 RPM for 2 minutes at 4° C. and place the plate on dry ice;
17. Store plates in −80° C. freezer.

The results indicated that there were almost no cells that passed quality control.

At the next sample, the protocol was modified by using different enzymes and shortening the times of incubation and of the physical dissociation. This allows a gentler treatment to the tissue. The results for prostate cancer samples are presented in FIG. 34.

The modified protocol includes the following:
1. Tumor is transported to the lab in PBS and on ice;
2. Cut sample tissue into tiny cubes (1×1 mm³) using scalpels;
3. Make digestion buffer—remove 2 ml M199 medium from 37° C. water bath and add 2 μl collagenase IV (100 mg/ml) and 20 ul DNase I (1 μg/μl);
4. Using scalpels, transport tumor cubes to media in a 15 ml Falcon tube, use 1 ml digestion buffer to wash off cells from dish and add to the 15 ml tube;
5. Place 15 ml tube in 37° C. water bath for 5 min;
6. Remove tube, vortex on maximum speed for 5 seconds;
7. Use 5 ml followed by 2 ml pipette to pipette up and down (5 times) and repeat using 1000 μl pipette tip;
8. Place tube on ice. Using a 100 μm mesh, filter solution into new 15 ml Falcon tube;
9. Wash filter with 10 ml PBS+2% FCS;
10. Spin at 580G×5 minutes at 4° C., remove supernatant;
11. Resuspend in 2 ml PBS+2% FCS;
12. Staining protocol: prepare following tubes on ice:
   a. No stain control: 200 μl unstained cell solution;
   b. Calcein-AM: 200 μl cell solution+1 μl Calcein;
   c. CD45-FITC: 200 μl cell solution+1 μl CD45-FITC;
   d. EPCAM-PE: 200 μl cell solution+4 μl EPCAM-PE;
   e. Sample: 1200 μl cell solution+6 μl Calcein+6 μl CD45−+24 μl EPCAM;
13. Let calcein single color control and sample tube incubate at RT for 10-15 minutes, then place back on ice with other tubes;
14. Proceed to FACS, use 96-well plates containing 10 μl of lysis buffer (TCL buffer+1% beta Mercapto EtOH) in each well; sort viable cells (calcein positive) that are CD45 positive (immune cells) or CD45 negative and EpCam positive (cancer cells);
15. When sorting finished immediately seal the plate, vortex vigorously for 10 seconds, spin down at 3700 RPM for 2 minutes at 4° C. and place the plate on dry ice;
16. Store plates in −80° C. freezer.

### Colon Cancer Samples

Colon samples were processed initially using the following protocol:
1. Tumor is transported to the lab in PBS and on ice;
2. Cut sample tissue into tiny cubes (1×1 mm³) using scalpels;
3. Make digestion buffer—remove 5 ml M199 medium from 37° C. water bath and add collagenase P (2 mg/ml) and DNase I (10 μg/μl);
4. Using scalpels, transport tumor cubes to media in a 15 ml Falcon tube, use 1 ml digestion buffer to wash off cells from dish and add to the 15 ml tube;
5. Place 15 ml tube in 37° C. water bath for 10 min;
6. Remove tube, vortex on maximum speed for 10 seconds;
7. Use 5 ml followed by 2 ml pipette to pipette up and down (at least 10 times) and repeat using 1000 μl pipette tip;
8. Repeat steps 6-7;
9. Place tube on ice. Using a 70 μm mesh, filter solution into new 15 ml Falcon tube;
10. Wash filter with 10 ml PBS+2% FCS;
11. Spin at 580G×5 minutes at 4° C., remove supernatant;
12. Send sample for 10× procedure—20,000 cells in 100 ul PBS 0.04% bovine serum albumin (BSA).

Due to the low number of cells that passed the quality control and qualified as successful cells, the process of colon tumor dissociation was modified. Enzymes were replaced with AccuMax, times and resuspensions were reduced, filter and nuzzle were replaced to 100 μl and calcein was replaced with 7AAD. The results for colon cancer samples are presented in FIG. 35-FIG. 38. The revised protocol is set forth below.

1. Tumor is transported to the lab in PBS and on ice;

2. Cut sample tissue into tiny cubes (1×1 mm³) using scalpels;

3. AccuMax 3 ml, 10 minutes at RT., rocking table;

4. Remove tube, vortex on maximum speed for 5 seconds;

5. Use 5 ml pipette followed by 1 ml tip to pipette up and down (5 times) on ice;

6. Place tube on ice. Using a 100 μm mesh, filter solution into new 15 ml Falcon tube;

7. Wash filter with 20 ml PBS+2% FCS;

8. Spin at 580G×5 minutes at 4° C., remove supernatant;

9. Resuspend in PBS 0.04% BSA;

10. Send sample for 10× procedure—20,000 cells in 100 ul PBS 0.04% BSA.

### Pancreas Cancer Samples

For pancreas samples the same protocol for all 3 samples was applied:

1. Tumor is transported to the lab in PBS and on ice;

2. Cut sample tissue into tiny cubes (1×1 mm³) using scalpels;

3. AccuMax 3 ml, 10 minutes in RT., rocking table;

4. Remove tube, vortex on maximum speed for 5 seconds;

5. Use 5 ml pipette followed by 1 ml tip to pipette up and down (5 times) on ice;

6. Place tube on ice. Using a 100 μm mesh, filter solution into new 15 ml Falcon tube;

7. Wash filter with 20 ml PBS+2% FCS;

8. Spin at 580G×5 minutes at 4° C., remove supernatant;

9. Send sample for SeqWell procedure-100,000 cells in 1 ml RPMI 10% FCS.

It was identified that at step 2 it is better to produce tissue pieces smaller than 1 cm. Cutting the tissue to smaller pieces increases the number of viable cells and statistically favors viable cells over dying cells that are easily shaded form the tissue. The results for pancreas cancer samples are presented in FIG. 39.

### Ovarian Cancer Samples

This protocol was improved using samples from the same patient, taken at different times (DF3250 from 7-1-16 and from 8-10-16); liquid samples from ovarian patients were taken from the abdominal ascites and were processed for single cell RNA-seq. Ovarian cancer cells (OvCa) were noticed at <50% of the total cell population. The protocol that was used to processes the earlier sample was the following:
1. Tumor is transported to the on ice;
2. Distribute 300 ml into 50 ml canonical tubes, move on ice;
3. Spin down tubes at 580G 6 min. at 4° C.;
4. Remove the supernatant and resuspended cells in 5 ml ACK (red blood cells lysis);
5. Incubate the sample on ice for 3 minutes and centrifuged 580 6 min. at 4° C.;
6. Repeat step 5 for 3 times or more (until the red rim is gone);
7. Resuspended the cell pellet in a total volume of 10 ml PBS+2% FCS;
8. Filter the cell suspension using a 100 μm mesh;
9. Reduce immune cell population by MACS (a magnetic sorter kit from Miltenyi Biotec, San Diego, Calif.);
10. Resuspend 20,000 cells in PBS 0.04% BSA and submit to 10× procedure.

After a modification was introduced to the protocol, the percentage of OvCa cells increased to >60%. The single modification was the ACK treatment (lysis of red blood cells) that was reduced to 2 cycles from the previous protocol (3 or more cycles). The ACK reagent might be somewhat toxic to ovarian non-RBCs and impair viability or RNA stability. The results for ovarian cancer samples are presented in FIG. 40A-FIG. 40B.

Finally, a QC step was added before submitting cells for 10× and SeqWell procedures; cells were incubated with trypan blue reagent and observed by light microscope. If viable cells were >60%, the cells were submitted. However, if the cells showed poor viability, the dead cell number was reduced by gently resuspending the cells in cold PBS, spinning down at 580G for 3 min. at 4° C. and counting viable cells again.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of disaggregating a tissue sample into a population of single cells in about one hour or less total, the method comprising:
dissecting said tissue sample into pieces;
enzymatically disaggregating said tissue sample for about 1 minute to about 20 minutes; and
mechanically disaggregating said tissue sample by pipetting said tissue sample up and down for about 30 seconds to about 5 minutes, thereby disaggregating said tissue sample into a population of single cells in about one hour or less, wherein at least 50% to 100% of said single cells are viable and retain surface markers.

2. A method of disaggregating a tissue sample into a population of single cells comprising
dissecting said tissue sample into pieces;
enzymatically disaggregating said tissue sample; and
mechanically disaggregating said tissue sample,
thereby disaggregating said tissue sample into a population of single cells.

3. The method of claim 2, wherein said tissue sample is dissected into pieces <1 mm³.

4. The method of claim 3, wherein said tissue sample is dissected with a scalpel.

5. The method of claim 2, wherein said tissue sample is enzymatically disaggregated with collagenase P and DNase I for about 10 minutes at about 37° C.

6. The method of claim 2, wherein said tissue sample is mechanically disaggregated by pipetting said tissue sample up and down for 1 minute with pipettes of descending sizes.

7. The method of claim 6, wherein said pipette comprises a 25 ml, 10 ml, 5 ml, and 1 ml pipette.

8. The method of claim 7, wherein said tissue sample is mechanically disaggregated by pipetting said tissue sample up and down for 2 additional minutes with pipettes of descending sizes

9. The method of claim 2, wherein said method further comprises removing red blood cells from said tissue sample.

10. The method of claim 2, wherein said method further comprises filtering said tissue sample and discarding residual cell clumps.

11. The method of claim 2, wherein said pipette diameter is progressively decreased with a removable pipette tip adapter.

12. The method of claim 2, wherein said pipette comprises an internal surface comprising teeth which mechanically shred said tissue sample.

13. The method of claim 2, wherein said population of cells comprises a single cell suspension.

14. The method of claim 2, wherein said method is performed in less than 5 hours.

15. The method of claim 2, wherein at least 50% of said single cells are viable;
wherein said method does not alter, remove, or add single cell surface markers; or
wherein said tissue sample comprises cancer tissue, non-cancerous diseased tissue, or healthy normal tissue.

16.-17. (canceled)

18. The method of claim 17, wherein said tissue sample is derived from a melanoma, ovarian cancer, breast cancer, colorectal cancer, pancreatic cancer, lung cancer, head and neck cancer, or prostate cancer.

19. The method of claim 18, wherein said tissue sample comprises a solid tumor, a core needle biopsy, a fine needle aspiration, a malignant effusion, a bone marrow aspirate, or a blood sample.

20. The method of claim 2, wherein said tissue sample is a human or a mouse tissue sample;
wherein said tissue sample comprises solid tissue, spheroid tissue, or a single cell solution;
wherein said single cells comprise tumor cells, T-cells, B-cells, NK-cells, macrophages, dendritic cells, cancer-associated fibroblasts, or endothelial cells; or
further comprising performing single-cell RNA-seq on said sample.

21.-23. (canceled)

24. A kit comprising collagenase P, DNase I, and a pipette tip.

25. The kit of claim 24, wherein said kit comprises a 25 ml pipette tip, a 15 ml pipette tip, a 10 ml pipette tip, a 5 ml pipette tip, and a 1 ml pipette tip;
wherein said kit further comprises a series of pipette tip adapters, wherein said pipette tip diameter is decreased;
wherein said kit comprises a pipette tip comprising an internal surface comprising teeth for use in shredding a tissue sample; or
wherein said kit further comprises a scalpel.

26.-28. (canceled)
